(19) United States
(12) Patent Application Publication
(10) Pub. No.: US 2011/0245153 A1
(43) Pub. Date: Oct. 6, 2011

(54) Neutralizing Agents for Bacterial Toxins

(75) Inventors: David M. Kranz, Champaign, IL (US); Rebecca A. Buonpane, Gaithersburg, MD (US); Hywyn R.O. Churchill, Radford, VA (US); Eric J. Sundberg, Somerville, MA (US); Beenu Moza, Cambridge, MA (US); Patrick Schlievert, Edina, MN (US)

(73) Assignee: National Institutes of Health (NIH), U. S. Dept. of Health and Human Resources (DHHS) U. S. Govt., Bethesda, MD (US)

(21) Appl. No.: 12/282,193

(22) PCT Filed: Mar. 15, 2007
(86) PCT No.: PCT/US2007/064085

§ 371 Date: Jul. 24, 2009

Related U.S. Application Data
(60) Provisional application No. 60/782,708, filed on Mar. 15, 2006.

(51) Int. Cl. A61K 38/17; A61P 31/04; C40B 30/04; C07K 14/725

(52) U.S. Cl. 514/2.7; 514/2.4; 506/9; 530/350

(57) ABSTRACT

Stabilized variable regions of the T cell receptor and methods of making the same using directed evolution through yeast display are provided. In one embodiment, the variable region is variable beta. In one embodiment, the stabilized T cell receptor variable regions have high affinity for a superantigen, such as TSST-1 or SEB. These T cell receptor variable regions are useful as therapeutics.

### CROSS-REFERENCE TO RELATED APPLICATIONS

This application claims priority to U.S. provisional application 60/782,708, filed Mar. 15, 2006, which is incorporated by reference to the extent not inconsistent with the disclosure herewith.

### STATEMENT REGARDING FEDERAL FUNDING

This invention was made with U.S. Government support under Grant number R01AI064611 awarded by the National Institutes of Health. The U.S. Government has certain rights in the invention.

### BACKGROUND OF THE INVENTION

Toxic shock syndrome (TSS) was characterized as a disease associated with staphylococci infection over 25 years ago. Subsequently, toxic shock syndrome toxin-1 (TSST-1) from Staphylococcus aureus was identified as the protein responsible for the disease in most cases. TSST-1 is a member of a family of molecules secreted by S. aureus and Streptococcus pyogenes that cause elevated systemic cytokine levels, including tumor necrosis factor-α (TNF-α) and interleukin-1 (IL-1), leading to fever, TSS, and ultimately organ failure. The term superantigen (SAg) was given to this class of molecules because these toxins stimulate a large fraction of T cells bearing the same variable regions of the T cell receptor beta chain (Vβ regions). As up to 20% of the T cell repertoire can bear the same Vβ region, SAgs are capable of stimulating thousands of times more T cells than conventional antigens. Since soluble monovalent ligands for the T cell receptor (TCR) cannot themselves stimulate T cells, SAgs act by cell-to-cell cross-linking TCRs and class II major histocompatibility complex (MHC) molecules on antigen presenting cells.

The bacterial SAg family now contains over 20 members, including the S. aureus enterotoxins TSST-1, (SE) A to E, and G to Q and the S. pyogenes exotoxins A (Spe) A, C, G to M, and the mitogenic exotoxins called SMEZ. Sequence based phylogenetic relationships among these toxins indicated that they represent five groups, in which one group contains TSST-1 as the only known member. The structures of SAgs, including TSST-1, have been shown to be very similar. A smaller N-terminal domain contains two β-sheets and a larger C-terminal domain consists of a central α-helix and a five-stranded β-sheet. Although all bacterial SAgs share a common three-dimensional structure, they exhibit diversity in their specificities for TCR Vβ domains and class II MHC molecules, as well as in the molecular architecture of the respective MHC-SAg-TCR signaling complexes that they form.

These superantigens cause many diseases, including pneumonia, mastitis, phlebitis, meningitis, urinary tract infections; osteomyelitis, endocarditis, nosocomial infection, staphylococcal food poisoning and toxic shock syndrome. Current treatments include supportive care, antibiotics, and intraveneous immune globulin. There are several strains of S. aureus that are antibiotic resistant.

Staphylococcal enterotoxin B (SEB), one of the more thoroughly characterized SAgs, has been considered a potential biological weapon due to its toxicity and to previous programs involving large-scale production and aerosolization.

Despite the fact that the molecular interactions of these toxins have been well-characterized, therapeutics capable of neutralizing their activity are not available for clinical use. There is a need in the art for a therapeutic agent to treat superantigen-mediated disease.

### SUMMARY OF THE INVENTION

Provided are methods for making a stabilized T cell receptor variable region, comprising: (a) cloning the T cell receptor variable region gene in a yeast display vector; (b) mutagenizing the T cell receptor variable region to generate a library of mutants; and (c) selecting the mutants which have the highest binding affinity to a ligand. Steps (b) and (c) can be repeated as desired, in order to obtain a T cell receptor variable region having the desired stability. In separate embodiments, the T cell receptor variable region is selected from the group consisting of Vα, Vβ, Vγ, and Vδ. In one embodiment, the T cell receptor variable region is a human Vβ. The ligand can be any desired ligand, including an antigen or superantigen. In one embodiment, the ligand is an antibody for the T cell receptor variable region. In one embodiment, the ligand is a superantigen. In one embodiment, the ligand is TSST-1. In one embodiment, the ligand is SEB.

In a specific embodiment, the T cell receptor variable region is hVβ2. In a specific embodiment, the T cell receptor variable region is mVβ8.

Also provided is a stabilized T cell receptor variable domain comprising: a T cell receptor variable region which contains one or more mutations wherein the stabilized T cell receptor variable domain binds with greater affinity to a ligand than wild type. In a specific embodiment, the variable domain is hVβ. In a specific embodiment, the variable domain contains at least one mutation selected from the group consisting of: S88G, R10M, A13V, L72P, and R113Q. In a specific embodiment, the variable domain is mVβ8, and the variable domain contains the mutation G17E and optionally one or more mutations selected from the group consisting of: N24K, G42E, H47F, Y48M, Y50H, A52I, G53R, S54N, and T55V. Any mutation or combination of mutations described or shown that gives a stabilized T cell receptor variable region is intended to be disclosed separately. Any mutation or combination of mutations described or shown that gives a higher affinity mutant is disclosed separately.

Also provided is a method for using stabilized T cell receptor variable region to select mutants that bind to a ligand or molecule of interest with higher affinity than wild type comprising: providing a stabilized T cell receptor variable region; mutating the stabilized T cell receptor variable region to create a variegated population of mutants; contacting the variegated population of mutants with a ligand; and selecting those mutants which bind to the ligand with higher affinity than wild type. In one embodiment, the mutant and ligand bind with an equilibrium binding constant K_{D}<1 μM. In one embodiment, the mutant and ligand bind with an equilibrium binding constant K_{D}<10 μM. In one embodiment, the mutant and ligand bind with an equilibrium binding constant K_{D}<10 nM. In one embodiment, the mutant and ligand bind with an equilibrium binding constant K_{D}<100 pM. In one embodiment, the mutant and ligand bind with an equilibrium binding constant K_{D}<10 pM. In one embodiment, the mutant and ligand bind with an equilibrium binding constant K_{D}<100 nM. In one embodiment, the mutant and ligand bind with an equilibrium binding constant K_{D}<1 nM. All individual values and intermediate ranges of equilibrium binding constants less than 100 μM are included herein, including specifically for the purpose of use in the claims to exclude prior art.

Also provided is a soluble mutant T cell receptor (TCR) variable region having higher affinity than the wild type T cell receptor variable region for a bacterial superantigen, wherein said T cell receptor variable region is a mutant T cell receptor variable region carrying one or more mutations in a TCR variable region. In one embodiment, the TCR variable region exhibits an equilibrium binding constant K_{D} for the bacterial superantigen of between about 10⁻⁸M and 10⁻¹²M. In one embodiment, the TCR variable region is a mutant TCR having one or more mutations in a CDR. In one embodiment, the TCR variable region is a mutant TCR having one or more mutations in a FR region. In one embodiment, the bacterial superantigen is toxic shock syndrome toxin-1. In one embodiment, the TCR variable region has one or more mutations in the human Vβ2 region. In one embodiment, the TCR variable region has one or more mutations in the Vβ2.1 region. In one embodiment, the TCR variable region has one or more mutations in CDR2. In one embodiment, the bacterial superantigen is staphylococcal enterotoxin B. In one embodiment, the TCR variable region has one or more mutations in the mouse Vβ8 domain. In one embodiment, the TCR variable region has one or more mutations in the Vβ8.2 domain. In one embodiment, the variable region is selected from Seq. ID Nos. 16-22; 30-44; and 66-73.

Also provided is a method for treating staphylococcus infection in a mammal, the method comprising: providing an effective amount of a high affinity mutant TCR variable region having one or more mutations in the TCR variable beta region, which TCR variable region binds to the superantigen with higher affinity than wild type TCR variable region, wherein the high affinity TCR variable region interferes with the binding of the superantigen to the MHC class II molecules and T cell receptors of the mammal.

Also provided is a method of treating a disease state in a mammal caused by a bacterial superantigen comprising: administering an effective amount of a high affinity mutant of the T cell receptor variable region to a mammal. In one embodiment, the mammal is a human. In one embodiment, the variable region is a variable beta region. In one embodiment, the disease is selected from the group consisting of: pneumonia, mastitis, phlebitis, meningitis, urinary tract infections; osteomyelitis, endocarditis, nosocomial infection, staphylococcal food poisoning and toxic shock syndrome. In one embodiment, the T cell receptor variable region is selected from Seq. ID Nos. 16-22; 30-44; and 66-73.

Also provided is a therapeutic composition comprising a stabilized T cell receptor variable region and optional pharmaceutical additives.

Provided are compositions comprising soluble protein domains of the T cell receptor variable region that have high-affinity for a ligand, and methods for preparation thereof. In one embodiment, the ligand is a superantigen. The compositions bind to the active site of the superantigen and prevent or decrease the normal effect of the superantigen. These compositions are useful as therapeutics for those animals, including mammals, including humans, which are affected by a disease caused by the superantigen.

The compositions of the invention are prepared and selected using yeast display techniques described in detail elsewhere. Generally, a library of mutants of the protein of interest are displayed on yeast cells and labeled with fluorescently labeled antibodies. The library is screened and those yeast cells displaying mutants which bind to the desired ligand with higher affinity are selected. The selected mutants can be mutagenized and screened for as many rounds as desired or required to provide the mutant with a desired affinity.

Regions and positions for site-directed mutagenesis of the T cell receptor variable region may be determined by selecting portions of the T cell receptor variable region that are believed to contact the superantigen (“contact regions”). These contact regions can be determined by structural models or calculations, as known in the art. For the systems described herein, the contact regions are primarily in the CDR2 and framework (FR) regions.

The compositions described herein are about 12,000 daltons, although larger or smaller compositions are included in this invention and prepared by one of ordinary skill in the art without undue experimentation.

As used herein, a “stabilized” protein means the protein is displayable on yeast. As shown previously, wild type single-chain T cell receptor domains are not displayable on yeast, and require at least one mutation to display the properly folded protein. (PNAS 96:5651 (1999); J. Mol. Biol. 292:949 (1999); Nature Biotech. 18:754 (2000)). The mutation may be in any region or regions of the variable domain that results in a stabilized protein. In one embodiment, one or more mutations is in one or more of CDR1, CDR2, HV4, CDR3, FR2, and FR3. The regions used for mutagenesis can be determined by directed evolution, where crystal structures or molecular models are used to generate regions of the TCR which interact with the ligand of interest (toxin or antigen, for example). In other examples, the variable region can be reshaped, by adding or deleting amino acids to engineer a desired interaction between the variable region and the ligand.

The yeast display cloning vector used in these experiments can be any vector which allows insertion of the mutated protein and display on yeast. One particular example of a yeast display cloning vector is pCT202, which is shown in FIG. 1C. The use of this vector has been described previously. The mutations that allow surface display also yield thermally stable, soluble variable region domains that can be secreted from yeast.

This invention provides a method for making stabilized T cell receptor (TCR) variable domains. These stabilized TCR variable domains are useful as receptor antagonists for ligands such as SEB, TSST-1, and SEC3. The methodology exemplified in the examples can be used to make stabilized TCR variable domains for any antigen. The terms “variable region” and “variable domain” are used interchangeably.

In one embodiment, stabilized proteins for TSST-1 are hVβ2.1 regions with one or more of the mutations S88G, R10M, A13V, L72P, and R113Q. In one embodiment, neutralizing agents for TSST-1 include those clones having the sequences exemplified with designations C4, C8, C10, D9, D10, D19, and D20 in FIG. 2. In one embodiment, neutralizing agents for TSST-1 have more than 5000 times increase in affinity for the toxin than the wild type. In one embodiment, stabilized proteins for SEB are mVβ8.2 regions with the mutation G17E and optionally one or more mutations selected from the group consisting of: N24K, G42E, H47F, Y48M, Y50H, A52I, G53R, S54N, and T55V. In one embodiment, neutralizing agents for SEB include those clones having the sequences exemplified with designations G5-x (x=3, 4, 6, 8, 9, 10, 11, 15) in FIG. 23. In one embodiment, neutralizing agents for SEB have more than 5000 times increase in affinity for the toxin than the wild type. All variable region sequences that are stabilized are individually included in this disclosure. All variable region sequences given here that have higher affinity for a ligand than a wild type sequence are individually included in this disclosure.

Therapeutic products can be made using the materials shown herein. Effective amounts of therapeutic products are the minimum dose that produces a measurable effect in a subject. Therapeutic products are easily prepared by one of ordinary skill in the art. In one embodiment, the variable domain is administered directly to a patient. In one embodiment, the variable domain is linked to an immunoglobulin constant region and used as a therapeutic. This embodiment extends the lifetime of the variable domain in the serum. In one embodiment, the variable domain is linked to PEG, as known in the art. This embodiment lengthens the serum clearance. These methods and other methods of administering, such as intravenously, are known in the art. Useful dosages are easily determined by one of ordinary skill in the art.

Mutagenesis methods used here include the use of mutator strains of E. coli, error-prone PCR, site-directed mutagenesis with degenerate primers/PCR, DNA shuffling, and other methods known in the art. Cloning methods used include standard ligations and electroporation, and homologous recombination of PCR products. Library sizes of up to 10⁷ molecules, for example, are formed. One method of analysis, fluorescent-activated cell sorting has been described previously.

In the methods for making neutralizing agents described herein, a stabilized T cell receptor variable region is used as the starting material for additional rounds of mutations and sorting. This process gives neutralizing agents with increasingly higher affinity to a toxin or antigen of interest. As used herein, “neutralizing agent” is a protein or protein fragment which binds to a molecule of interest with greater affinity than a wild type protein or protein fragment and is also referred to as “high affinity.” In one embodiment, the neutralizing agent has an affinity for the molecule of interest of more 5,000 times that of the wild type. In one embodiment, the neutralizing agent has an affinity for the molecule of interest of more 10,000 times that of the wild type. In one embodiment, the neutralizing agent has an affinity for the molecule of interest of more than 100,000 times that of wild type. Herein, the usage of the terms dissociation constant and equilibrium binding constant are consistent with the usage in the art and the context given.

In the figures and tables which present amino acid sequences, the wild type is designated “WT”. In the sequences presented below the top sequence, a dash indicates the amino acid is the same as the top sequence. A letter indicates a substitution has been made in that position from the top sequence.

In one embodiment of the invention, administration of an effective amount of a neutralizing agent is useful in preventing or reducing the toxic effects of a bacterial superantigen. In one embodiment of the invention, administration of an effective amount of a neutralizing agent prevents or reduces the binding of a bacterial superantigen to the variable region. In one embodiment of the invention, administration of an effective amount of a neutralizing agent prevents or reduces the crosslinking of the variable region and MHC.

### BRIEF DESCRIPTION OF THE DRAWINGS

FIG. 1. Yeast display of human Vβ2.1 before and after stabilization. (a) Yeast display construct of hVβ2.1 (Aga2/HA/h Vβ2.1/c-myc). (b) Yeast cell histograms of wild-type hVβ2.1 and clone EP-8 isolated from the error-prone library after staining with an anti-human Vβ2 antibody. (c) Yeast display vector (GAL1-10) (AGA2/HA) (NheI) (2CscTCR [VβII Vα]) (6-His) (XhoI).

FIG. 2. Sequences of some hVβ2.1 mutants isolated in the yeast display system. The designation EP refers to clones isolated from the error-prone (stability) library. The designation R refers to clones isolated from the CDR2 (affinity) library. The designation C or D refer to clones isolated from the third and fourth sorts, respectively, from the combined CDR1, CDR2b, or HV4 (off-rate) library.

FIG. 3. Binding of TSST-1 to affinity matured hVβ2.1 mutants. (a) Overlay histogram of the stabilized human Vβ2.1 clone, EP-8 (black outline), and a clone from the first-generation affinity library, R9 (gray). Yeast cells were incubated with 200 nM biotinylated TSST-1, and analyzed by flow cytometry. (b) A panel of clones isolated from the first generation library were incubated with 200 nM biotinylated TSST-1 and analyzed by flow cytometry to determine their relative fluorescence (mean fluorescence units, MFU). Inset: a representative equilibrium binding titration of biotinylated-TSST-1 to clone R9. The x-axis represents the TSST-1-biotin concentration in nanomolar, and the y-axis represents the MFU of the samples.

FIG. 4. Binding of TSST-1 to affinity matured, second generation hVβ2.1 mutants. Analysis of the second-generation clones selected from the combined CDR1/CDR2b/HV4 libraries. (a) Equilibrium binding of clones isolated from the third (C1-10) and fourth (D1-20) rounds of sorting. Clones were incubated with 5 nM biotinylated TSST-1 followed by SA/PE and analyzed by flow cytometry. R9 is also shown, as well as EP-8. (b) Clones were incubated with 5 nM biotinylated TSST-1 for 1 h under equilibrium conditions, and then incubated with a tenfold molar excess of unlabeled TSST-1 for 2 h at 25° C. A sample was removed before the unlabeled TSST was added and placed on ice until the end of the experiment. Percent remaining bound was calculated as (MFU after 2 h at 25° C./MFU at time zero)×100. R9 is also shown.

FIG. 5. Off-rate analysis of TSST-1 binding to selected hVβ2.1 clones. (a) Overlay histogram demonstrating the percent biotinylated TSST-1 remaining bound to clone C10. The off-rate of clone C10 was examined by incubating the clones with 5 nM biotinylated TSST-1 for 1 h on ice, followed by incubation with a 50-fold molar excess of unlabeled TSST-1 at 37° C. Time points were taken after 0 h, 12 h, and 24 h at 37° C. (b) Off-rate time points of first generation clones (R9 and R18) and second generation clones (C4, C10 and D10) were examined using the same experimental design as in (a).

FIG. 6. Binding of TSST-1 to single-site alanine mutants of hVβ2.1 clone C10. The position of the mutant is shown in the x-axis. (a) Equilibrium binding of alanine mutants to c-myc antibody (which is a measure for the amount of folded protein on the cell surface; data not shown) and 5 or 20 nM biotinylated TSST-1 were used to determine the mean fluorescence units of binding. The 5 nM data is shown in the right in each mutant column—the 20 nM data is shown to the left in each mutant column. These values for c-myc and TSST-1 were used to calculate the ratio. (b) Examination of the percent biotinylated TSST-1 remaining bound after 2 h. Cells were incubated with 5 nM biotinylated TSST-1 for 1 h on ice, followed by a 50-fold molar excess of unlabeled TSST-1 for 2 h at 37° C. A sample of the yeast was removed before transferring to elevated temperature. The line on FIG. 6B indicates the percent of TSST-1 remaining bound to clone C10. First generation clones R9, R17, and R18 are included for comparison

FIG. 7. SPR analysis of the interactions between hVβ2.1 variants and immobilized TSST-1. The inset in (a) depicts the Scatchard analysis of equilibrium binding between EP-8 with TSST-1. Global fitting of data ((b)-(f)) to a 1:1 binding model is shown in black.

FIG. 8. Competition between TSST-1 and SpeC for binding to hVβ2.1. (a) SpeC was immobilized on biosensorchip, and the stabilized hVβ2.1 mutant EP-8 was injected at various concentrations (0.39 to 100 μM) over the chip. (b) EP-8 at 12.5 μM was incubated with various concentrations of TSST-1 (0 to 100 μM) and the mixtures were injected over the chip with immobilized SpeC. (c) EP-8 at 12.5 μM was incubated with various concentrations of the SAg SEB (0 to 100 μM) and the mixtures were injected over the chip with immobilized SpeC.

FIG. 9. Model of the hVβ2.1-C10 and TSST-1 interaction. (a) Model of mutant hVβ2.1-C10 based on the structure of the wild-type human Vβ2.1. The 013 is included in the model for orientation. Mutations that were isolated during the screening for yeast displayed hVβ2.1 are shown. (b) Hypothetical model of the hVβ2.1-C10-TSST-1 complex. Mutated residues that were isolated during screening for higher affinity are shown (CDR2, K62 and Y56). (c) The crystal structure of human Vβ2.1 in complex with the superantigen SpeC (PDB accession code 1KTK). The Vβ domain is shown in the same orientation as in the hVβ2.1-C10-TSST-1 model for comparison.

FIG. 10. Equilibrium binding analysis of single-site variants. (A) The changes in free energy for each of the single-site hVβ2.1 mutants binding to TSST-1 are plotted. The dotted line indicates the threshold value used to distinguish energetically significant versus insignificant mutations. Equilibrium and/or kinetic binding analysis of (B) EP-8 and the (C) T30H, (D) E51Q, (E) S52aF, (F) K53N and (G) E61V mutants interacting with TSST-1 for which SPR sensorgrams, after correction for non-specific binding, are shown. Inset plots in panels (B)-(F) show non-linear steady-state affinity analysis for the corresponding interaction. Global fitting of the data to a 1:1 binding model is shown in panels (E)-(G) in black and the corresponding residual values are plotted below the individual sensorgrams.

FIG. 11. Two hot regions for TSST-1 interaction in hVβ2.1. (A) The wild type side chains of each of the single-site mutations in the hVβ2.1 affinity maturation pathway from EP-8 to D10 are shown as ball-and-stick representations on the backbone of the wild type hVβ2.1 crystal structure (E. J. Sundberg et al. (2002) Structure 10:687-99). Two views of the molecule are shown, positioned approximately 90 degrees about the vertical axis of the page. (B) Similar representation of the hVβ2.1 domain as in (A).

FIG. 12. Additivity and cooperativity of binding free energy. (A) Additive ΔG_{b} (defined as ΣΔΔG_{b(single-site mutants)}) and experimentally determined ΔG_{b} values of analogous combinatorial mutations are plotted. (B) ΔG_{COOP} values (calculated as the difference between the summation of the changes in binding free energies of the single-site mutants and the experimental changes in binding free energies of the corresponding combinatorial mutant) are plotted. The threshold values for cooperativity (∥ΔG_{COOP}|≧0.5 kcal/mol) are indicated by the dotted lines. In both panels, asterisks indicate particular combinations of mutations that are cooperative. Intra-hot regional (CDR2 only) mutations are clustered at the bottom and inter-hot regional (CDR2 and FR3) mutations are clustered at the top of each graph.

FIG. 13. The protein core as an energetic sink. Strand-swapping of the c″ β-strand in TCR Vβ domains as depicted in the (A) hVβ2.1 domain (E. Sundberg et al. (2002) Structure 10:687-99) and (B) the mVβ2.3 domain (D. Housset et al. (1997) Embo J 16:4205-16). (C) A view of the hVβ2.1 domain in which the protein core and the CDR2 and FR3 hot regions, and the connecting c″ Vβ-strand are outlined by dotted ovals on the left and right, respectively. Schematic models of (D) energetically cooperative hot regions connected by a structural element that does not form part of the protein core and of (E) energetically additive hot regions for which the connecting structural element forms part of the protein core. (F) Possible mechanisms for initiation of T cell signaling. A modified “pseudodimer” model is shown in which TCR molecules bind to both agonist and endogenous pMHC-II and the supramolecular complex is stabilized by the CD4 coreceptor. Asterisks indicate regions of the TCR V domain that exhibit long-range cooperative binding effects in the present study and bind pMHC-II (white asterisks) and potentially interact with CD4 and/or CD3 (black asterisks)

FIG. 14. Kinetic analysis of multi-site variants. SPR sensorgrams, after correction for non-specific binding, for the (A) D10, (B) S52aF/K53N/E61V, (C) E51Q/K53N and (D) E51Q/K53N/E61V mutants binding to TSST-1 are shown. Inset plot in (C) shows non-linear steady-state affinity analysis for the corresponding interaction. Global fitting of the data to a 1:1 binding model is shown all panels in black and the corresponding residual values are plotted below the individual sensorgrams.

FIG. 15 shows the sequences of mVβ8.2 mutants isolated for binding to SEB.

FIG. 16 shows binding of biotinylated SEB to yeast clones that express different Vβ8 mutants (where region CDR2 was mutated).

FIG. 17 shows titrations of biotinylated SEB and yeast expressing Vβ8 mutants (CDR2) to determine affinities. The K_{D} for EGIGYITK is ˜5 nM. The K_{D} for L2CM is ˜200 nM. The K_{D} for WT is ˜100 μM.

FIG. 18 shows binding of fifth generation clones to SEB. G4 is shown for comparison.

FIG. 19 shows off-rates of fourth generation (G4) and fifth generation (G5 m4-8) SEB-binding clones.

FIG. 20 shows surface plasmon resonance analysis of affinity matured mVb8.2 variants binding to SEB.

FIG. 21 shows reactivity to SEC3 of mVβ8.2 clones generated for high-affinity to SEB.

FIG. 22. Yeast display of Vβ8 for engineering SEB-binding mutants. (a) Yeast display construct of Vβ8. (b) Crystal structure of Vβ8 in complex with SEB Protein Data Bank (PDB) accession code 1SBB. Residues that contact the SEB molecule are shown in stick form. Location of the Vβ stabilizing residues G17 and G42 are shown. (c) Flow cytometry histogram of the wild-type Vβ8.2 (black) and the first generation clone G1-18 (gray). Yeast cells were incubated with 208 nM biotinylated SEB and analyzed by flow cytometry. (d) Fifth generation clones were incubated with 5 nM biotinylated SEB for one hour under equilibrium conditions, then incubated with a 10-fold molar excess of unlabeled SEB for 4 hours at 25° C. A sample was removed before the unlabeled SEB was added and placed on ice until the end of the experiment. Percent remaining bound was calculated as: (MFU after 4 hours at 25° C./MFU at time zero)×100.

FIG. 23. Sequences of Vβ8 mutants at the different stages of affinity maturation. G1 through G5 refers to the generation of clone isolated by yeast display. mTCR15 refers to a single-site mutant that has improved display on yeast, compared to the wild type Vβ8.2. CDR1, CDR2, HV4, and CDR3 regions are highlighted from left to right. Clones that were isolated multiple times are indicated with an asterisk.

FIG. 24. Binding analysis and in vitro inhibitory activity of soluble, high-affinity Vβ mutants. (a,b) Surface plasmon resonance analysis of affinity matured Vβ8. Representative SPR sensorgrams of Vβ mutants from generation two (G2-5)(a) and generation 5 (G5-8)(b). Two-fold dilutions (20 to 0.3125 nM) of Vβ mutants were analyzed for binding to immobilized SEB (533 RU). Dilutions of the Vβ8.2 variants are from top to bottom as follows: 20 nM; 10 nM; 5 nM; 2.5 nM; 1.25 nM; 0.625 nM; 0.3125 nM. (c,d) T cell inhibitory activity of Vβ mutants in T cell cytotoxicity assays. ⁵¹Cr-labeled Daudi cells were incubated for 4 hours with a 10:1 effector to target ratio of either 2C CTLs (c) or polyclonal CTLs (d) in the presence of 35 nM SEB and soluble Vβ antagonists: G5-8 (circles), G4-9 (squares), G2-5 (triangles), WT-mTCR15 (diamonds).

FIG. 25. Soluble Vβ blocks the activity and lethality of SEB in rabbits. (a) 5 μg/kg SEB and 500 μg/kg of the fifth generation clone G5-8 were pre-mixed at room temperature for one hour. 6 New Zeland white rabbits were injected with SEB alone (white bars) or the pre-mixed cocktail (black bars) and fever response was monitored. After 4 hours, the rabbits were challenged with 100-times the LD₅₀ of S. typhimurium LPS, and survival was monitored (b). Total number of rabbits that survived treatment is indicated over the bars. (c) The same experiment described in (a) and (b) was performed with various concentrations of the G5-8 Vβ or a high titer preparation of human IVIG (see text for details). Three rabbits were used at each dose and the percent survival was determined for each group.

FIG. 26. Soluble Vβ rescues rabbits exposed to SEB in the endotoxin enhancement or osmotic pump models. (a) 5 μg/kg SEB was administered to rabbits, followed 2 hours later by 500 μg/kg G5-8, and fever response was monitored. (b) Survival of rabbits challenged with 100× the LD₅₀ of S. typhimurium LPS. (c) 200 μg SEB was implanted subcutaneously in 2 groups of rabbits (3 rabbits per group) in Alza miniosmotic pumps. One group of rabbits was given 100 μg G5-8Vβ immediately after implanting the pumps, and then daily for 7 days; PBS was given to controls. Body temperature was monitored at the time of pump implantation (white bars) and after two days of treatment (black bars). (d) Survival analysis of rabbits over the span of 8 days.

FIG. 27. Analysis of Vβ8 mutants for SEB binding at different stages of affinity maturation. Yeast clones were incubated with various concentrations of biotinylated SEB and analyzed by flow cytometry. Mean fluorescence units (MFU) are from histograms of yeast clones incubated with SEB. Each bar represents an individual clone isolated from: (a) first generation, incubated with 208 nM SEB, (b) second generation, incubated with 100 nM SEB, (c) third generation, incubated with 10 nM SEB, (d) fourth generation, incubated with 1 nM SEB. Asterisks denote clones that were used as templates for the next generation of affinity engineering.

FIG. 28. Equilibrium SEB binding titration of clones at different stages of affinity maturation. (a) A representative clone from the first four generations was incubated with 5-fold dilutions of biotinylated SEB for one hour under equilibrium conditions and analyzed by flow cytometry. (b) Titrations of two second generation clones, and mutant L2CM. (c) Off-rate time points of a fourth (G4-9-circles) and fifth (G5-8-triangles) generation clone. Yeast clones were incubated with 5 nM biotinylated SEB for one hour on ice, followed by incubation for 2 hours at 37° C. in the presence of 50 nM unlabeled SEB. Aliquots were removed at the indicated time points, and labeled SEB remaining bound was measured by flow cytometry. (d) Serum lifetime of Vβ in mice. Mice were injected i.v. with soluble Vβ protein and at the indicated times, blood was drawn and serum was assayed by a competitive ELISA for the amount of Vβ.

FIG. 29. Surface plasmon resonance analysis of affinity matured Vβ8.2 clones. SPR sensorgrams of additional clones from generation 4: G4-9(a) and generation 5: G5-3 (b), G5-6 (c), G5-9 (d), and G5-10 (e). 2-fold dilutions (20 to 0.3125 nM) of variants binding to immobilized SEB (533 RU). Dilutions of the mVβ8.2 variants are from top to bottom as follows: 20 nM; 10 nM; 5 nM; 2.5 nM; 1.25 nM; 0.625 nM; 0.3125 nM.

FIG. 30. Serum clearance of ¹²⁵I-Vβ in the presence or absence of SEB. Four rabbits were administered ¹²⁵I-Vβ G5-8 (35.48×10⁶ cpm in 1 ml of PBS containing 1% normal rabbit serum). Two rabbits received 200 μg SEB in 1 ml PBS intravenously immediately prior to receiving Vβ, and two rabbits received 1 ml of PBS prior to receiving Vβ. Blood samples (0.1 ml) were drawn from the marginal ear veins of each rabbit at 30 seconds and then 5, 10, 20, 30, 60, 120, and 180 minutes after injection, and the average cpm of the samples from two rabbits of each cohort were plotted.

### DETAILED DESCRIPTION OF THE INVENTION

The following non-limiting description provides further illustration of some embodiments of the invention. Applicant does not wish to be bound by any theory presented here. The generation of high affinity T cell receptor variable regions for exemplary ligands, including TSST-1 and SEB, are demonstrated here. One of ordinary skill in the art would be able to produce high affinity T cell receptor variable regions for other superantigens and other ligands using the methods described here and methods known in the art without undue experimentation.

In order to provide a clear and consistent understanding of the specification and claims, including the scope to be given to such terms, the following definitions are provided.

A coding sequence is the part of a gene or cDNA which codes for the amino acid sequence of a protein, or for a functional RNA such as a tRNA or rRNA.

Complement or complementary sequence means a sequence of nucleotides which forms a hydrogen-bonded duplex with another sequence of nucleotides according to Watson-Crick base-pairing rules. For example, the complementary base sequence for 5′-AAGGCT-3′ is 3′-TTCCGA-5′.

Downstream means on the 3′ side of any site in DNA or RNA.

Expression refers to the transcription of a gene into structural RNA (rRNA, tRNA) or messenger RNA (mRNA) and subsequent translation of a mRNA into a protein.

An amino acid sequence that is functionally equivalent to a specifically exemplified TCR sequence is an amino acid sequence that has been modified by single or multiple amino acid substitutions, by addition and/or deletion of amino acids, or where one or more amino acids have been chemically modified, but which nevertheless retains the binding specificity and high affinity binding activity of a cell-bound or a soluble TCR protein of the present invention. Functionally equivalent nucleotide sequences are those that encode polypeptides having substantially the same biological activity as a specifically exemplified cell-bound or soluble TCR protein. In the context of the present invention, a soluble TCR protein lacks the portions of a native cell-bound TCR and is stable in solution (i.e., it does not generally aggregate in solution when handled as described herein and under standard conditions for protein solutions).

Two nucleic acid sequences are heterologous to one another if the sequences are derived from separate organisms, whether or not such organisms are of different species, as long as the sequences do not naturally occur together in the same arrangement in the same organism.

Homology refers to the extent of identity between two nucleotide or amino acid sequences.

Isolated means altered by the hand of man from the natural state. If an “isolated” composition or substance occurs in nature, it has been changed or removed from its original environment, or both. For example, a polynucleotide or a polypeptide naturally present in a living animal is not isolated, but the same polynucleotide or polypeptide separated from the coexisting materials of its natural state is isolated, as the term is employed herein.

A linker region is an amino acid sequence that operably links two functional or structural domains of a protein.

A nucleic acid construct is a nucleic acid molecule which is isolated from a naturally occurring gene or which has been modified to contain segments of nucleic acid which are combined and juxtaposed in a manner which would not otherwise exist in nature.

Nucleic acid molecule means a single- or double-stranded linear polynucleotide containing either deoxyribonucleotides or ribonucleotides that are linked by 3′-5′-phosphodiester bonds.

Two DNA sequences are operably linked if the nature of the linkage does not interfere with the ability of the sequences to effect their normal functions relative to each other. For instance, a promoter region would be operably linked to a coding sequence if the promoter were capable of effecting transcription of that coding sequence.

A polypeptide is a linear polymer of amino acids that are linked by peptide bonds.

Promoter means a cis-acting DNA sequence, generally 80-120 base pairs long and located upstream of the initiation site of a gene, to which RNA polymerase may bind and initiate correct transcription. There can be associated additional transcription regulatory sequences which provide on/off regulation of transcription and/or which enhance (increase) expression of the downstream coding sequence.

A recombinant nucleic acid molecule, for instance a recombinant DNA molecule, is a novel nucleic acid sequence formed in vitro through the ligation of two or more nonhomologous DNA molecules (for example a recombinant plasmid containing one or more inserts of foreign DNA cloned into at least one cloning site).

Transformation means the directed modification of the genome of a cell by the external application of purified recombinant DNA from another cell of different genotype, leading to its uptake and integration into the subject cell=s genome. In bacteria, the recombinant DNA is not typically integrated into the bacterial chromosome, but instead replicates autonomously as a plasmid.

Upstream means on the 5′ side of any site in DNA or RNA.

A vector is a nucleic acid molecule that is able to replicate autonomously in a host cell and can accept foreign DNA. A vector carries its own origin of replication, one or more unique recognition sites for restriction endonucleases which can be used for the insertion of foreign DNA, and usually selectable markers such as genes coding for antibiotic resistance, and often recognition sequences (e.g. promoter) for the expression of the inserted DNA. Common vectors include plasmid vectors and phage vectors.

High affinity T cell receptor (TCR) means an engineered TCR with stronger binding to a target ligand than the wild type TCR. Some examples of high affinity include an equilibrium binding constant for a bacterial superantigen of between about 10⁻⁸ M and 10⁻¹² M and all individual values and ranges therein.

It will be appreciated by those of skill in the art that, due to the degeneracy of the genetic code, numerous functionally equivalent nucleotide sequences encode the same amino acid sequence.

Additionally, those of skill in the art, through standard mutagenesis techniques, in conjunction with the assays described herein, can obtain altered TCR sequences and test them for the expression of polypeptides having particular binding affinity. Useful mutagenesis techniques known in the art include, without limitation, oligonucleotide-directed mutagenesis, region-specific mutagenesis, linker-scanning mutagenesis, and site-directed mutagenesis by PCR [see e.g. Sambrook et al. (1989) and Ausubel et al. (1999)].

In obtaining variant TCR coding sequences, those of ordinary skill in the art will recognize that TCR-derived proteins may be modified by certain amino acid substitutions, additions, deletions, and post-translational modifications, without loss or reduction of biological activity. In particular, it is well-known that conservative amino acid substitutions, that is, substitution of one amino acid for another amino acid of similar size, charge, polarity and conformation, are unlikely to significantly alter protein function. The 20 standard amino acids that are the constituents of proteins can be broadly categorized into four groups of conservative amino acids as follows: the nonpolar (hydrophobic) group includes alanine, isoleucine, leucine, methionine, phenylalanine, proline, tryptophan and valine; the polar (uncharged, neutral) group includes asparagine, cysteine, glutamine, glycine, serine, threonine and tyrosine; the positively charged (basic) group contains arginine, histidine and lysine; and the negatively charged (acidic) group contains aspartic acid and glutamic acid. Substitution in a protein of one amino acid for another within the same group is unlikely to have an adverse effect on the biological activity of the protein.

Homology between nucleotide sequences can be determined by DNA hybridization analysis, wherein the stability of the double-stranded DNA hybrid is dependent on the extent of base pairing that occurs. Conditions of high temperature and/or low salt content reduce the stability of the hybrid, and can be varied to prevent annealing of sequences having less than a selected degree of homology. For instance, for sequences with about 55% G-C content, hybridization and wash conditions of 40-50° C., 6×SSC (sodium chloride/sodium citrate buffer) and 0.1% SDS (sodium dodecyl sulfate) indicate about 60-70% homology, hybridization and wash conditions of 50-65° C., 1×SSC and 0.1% SDS indicate about 82-97% homology, and hybridization and wash conditions of 52° C., 0.1×SSC and 0.1% SDS indicate about 99-100% homology. A wide range of computer programs for comparing nucleotide and amino acid sequences (and measuring the degree of homology) are also available, and a list providing sources of both commercially available and free software is found in Ausubel et al. (1999). Readily available sequence comparison and multiple sequence alignment algorithms are, respectively, the Basic Local Alignment Search Tool (BLAST) (Altschul et al., 1997) and ClustalW programs. BLAST is available on the Internet at http://www.ncbi.nlm.nih.gov and a version of ClustalW is available at http://www2.ebi.ac.uk.

Industrial strains of microorganisms (e.g., Aspergillus niger, Aspergillus ficuum, Aspergillus awamori, Aspergillus oryzae, Trichoderma reesei, Mucor miehei, Kluyveromyces lactis, Pichia pastoris, Saccharomyces cerevisiae, Escherichia coli, Bacillus subtilis or Bacillus lichenifomis) or plant species (e.g., canola, soybean, corn, potato, barley, rye, wheat) may be used as host cells for the recombinant production of the TCR peptides. As the first step in the heterologous expression of a high affinity TCR protein or soluble protein, an expression construct is assembled to include the TCR or soluble TCR coding sequence and control sequences such as promoters, enhancers and terminators. Other sequences such as signal sequences and selectable markers may also be included. To achieve extracellular expression of the scTCR, the expression construct may include a secretory signal sequence. The signal sequence is not included on the expression construct if cytoplasmic expression is desired. The promoter and signal sequence are functional in the host cell and provide for expression and secretion of the TCR or soluble TCR protein. Transcriptional terminators are included to ensure efficient transcription. Ancillary sequences enhancing expression or protein purification may also be included in the expression construct.

Various promoters (transcriptional initiation regulatory region) may be used according to the invention. The selection of the appropriate promoter is dependent upon the proposed expression host. Promoters from heterologous sources may be used as long as they are functional in the chosen host.

Promoter selection is also dependent upon the desired efficiency and level of peptide or protein production. Inducible promoters such as tac are often employed in order to dramatically increase the level of protein expression in E. coli. Overexpression of proteins may be harmful to the host cells. Consequently, host cell growth may be limited. The use of inducible promoter systems allows the host cells to be cultivated to acceptable densities prior to induction of gene expression, thereby facilitating higher product yields.

Various signal sequences may be used according to the invention. A signal sequence which is homologous to the TCR coding sequence may be used. Alternatively, a signal sequence which has been selected or designed for efficient secretion and processing in the expression host may also be used. For example, suitable signal sequence/host cell pairs include the B. subtilis sacB signal sequence for secretion in B. subtilis, and the Saccharomyces cerevisiae α-mating factor or P. pastoris acid phosphatase phol signal sequences for P. pastoris secretion. The signal sequence may be joined directly through the sequence encoding the signal peptidase cleavage site to the protein coding sequence, or through a short nucleotide bridge consisting of usually fewer than ten codons, where the bridge ensures correct reading frame of the downstream TCR sequence.

Elements for enhancing transcription and translation have been identified for eukaryotic protein expression systems. For example, positioning the cauliflower mosaic virus (CaMV) promoter 1000 bp on either side of a heterologous promoter may elevate transcriptional levels by 10- to 400-fold in plant cells. The expression construct should also include the appropriate translational initiation sequences. Modification of the expression construct to include a Kozak consensus sequence for proper translational initiation may increase the level of translation by 10 fold.

A selective marker is often employed, which may be part of the expression construct or separate from it (e.g., carried by the expression vector), so that the marker may integrate at a site different from the gene of interest. Examples include markers that confer resistance to antibiotics (e.g., bla confers resistance to ampicillin for E. coli host cells, nptII confers kanamycin resistance to a wide variety of prokaryotic and eukaryotic cells) or that permit the host to grow on minimal medium (e.g., HIS4 enables P. pastoris or His⁻ S. cerevisiae to grow in the absence of histidine). The selectable marker has its own transcriptional and translational initiation and termination regulatory regions to allow for independent expression of the marker. If antibiotic resistance is employed as a marker, the concentration of the antibiotic for selection will vary depending upon the antibiotic, generally ranging from 10 to 600 μg of the antibiotic/mL of medium.

The expression construct is assembled by employing known recombinant DNA techniques (Sambrook et al., 1989; Ausubel et al., 1999). Restriction enzyme digestion and ligation are the basic steps employed to join two fragments of DNA. The ends of the DNA fragment may require modification prior to ligation, and this may be accomplished by filling in overhangs, deleting terminal portions of the fragment(s) with nucleases (e.g., ExoIII), site directed mutagenesis, or by adding new base pairs by PCR. Polylinkers and adaptors may be employed to facilitate joining of selected fragments. The expression construct is typically assembled in stages employing rounds of restriction, ligation, and transformation of E. coli. Numerous cloning vectors suitable for construction of the expression construct are known in the art (λZAP and pBLUESCRIPT SK-1, Stratagene, LaJolla, Calif.; pET, Novagen Inc., Madison, Wis.—cited in Ausubel et al., 1999) and the particular choice is not critical to the invention. The selection of cloning vector will be influenced by the gene transfer system selected for introduction of the expression construct into the host cell. At the end of each stage, the resulting construct may be analyzed by restriction, DNA sequence, hybridization and PCR analyses.

The expression construct may be transformed into the host as the cloning vector construct, either linear or circular, or may be removed from the cloning vector and used as is or introduced onto a delivery vector. The delivery vector facilitates the introduction and maintenance of the expression construct in the selected host cell type. The expression construct is introduced into the host cells by any of a number of known gene transfer systems (e.g., natural competence, chemically mediated transformation, protoplast transformation, electroporation, biolistic transformation, transfection, or conjugation) (Ausubel et al., 1999; Sambrook et al., 1989). The gene transfer system selected depends upon the host cells and vector systems used.

For instance, the expression construct can be introduced into S. cerevisiae cells by protoplast transformation or electroporation. Electroporation of S. cerevisiae is readily accomplished, and yields transformation efficiencies comparable to spheroplast transformation.

Monoclonal or polyclonal antibodies, preferably monoclonal, specifically reacting with a TCR protein at a site other than the ligand binding site may be made by methods known in the art. See, e.g., Harlow and Lane (1988) Antibodies: A Laboratory Manual, Cold Spring Harbor Laboratories; Goding (1986) Monoclonal Antibodies: Principles and Practice, 2d ed., Academic Press, New York; and Ausubel et al. (1999) Current Protocols in Molecular Biology, John Wiley & Sons, Inc., New York.

High affinity TCR proteins in cell-bound or soluble form which are specific for a particular superantigen are useful, for example, as diagnostic probes for screening biological samples (such as cells, tissue samples, biopsy material, bodily fluids and the like) or for detecting the presence of the superantigen in a test sample. Frequently, the high affinity TCR proteins are labeled by joining, either covalently or noncovalently, a substance which provides a detectable signal. Suitable labels include but are not limited to radionuclides, enzymes, substrates, cofactors, inhibitors, fluorescent agents, chemiluminescent agents, magnetic particles and the like. Additionally the TCR protein can be coupled to a ligand for a second binding molecules: for example, the TCR protein can be biotinylated. Detection of the TCR bound to a target cell or molecule can then be effected by binding of a detectable streptavidin (a streptavidin to which a fluorescent, radioactive, chemiluminescent, or other detectable molecule is attached or to which an enzyme for which there is a chromophoric substrate available). U.S. patents describing the use of such labels and/or toxic compounds to be covalently bound to the scTCR protein include but are not limited to Nos. 3,817,837; 3,850,752; 3,927,193; 3,939,350; 3,996,345; 4,277,437; 4,275,149; 4,331,647; 4,348,376; 4,361,544; 4,468,457; 4,444,744; 4,640,561; 4,366,241; RE 35,500; 5,299,253; 5,101,827; 5,059,413. Labeled TCR proteins can be detected using a monitoring device or method appropriate to the label used. Fluorescence microscopy or fluorescence activated cell sorting can be used where the label is a fluorescent moiety, and where the label is a radionuclide, gamma counting, autoradiography or liquid scintillation counting, for example, can be used with the proviso that the method is appropriate to the sample being analyzed and the radionuclide used. In addition, there can be secondary detection molecules or particle employed where there is a detectable molecule or particle which recognized the portion of the TCR protein which is not part of the binding site for the superantigen or other ligand in the absence of a MHC component as noted herein. The art knows useful compounds for diagnostic imaging in situ; see, e.g., U.S. Pat. Nos. 5,101,827; 5,059,413. Radionuclides useful for therapy and/or imaging in vivo include ¹¹¹Indium, ⁹⁷Rubidium, 125Iodine, ¹³¹Iodine, ¹²³Iodine, ⁶⁷Gallium, ⁹⁹Technetium. Toxins include diphtheria toxin, ricin and castor bean toxin, among others, with the proviso that once the TCR-toxin complex is bound to the cell, the toxic moiety is internalized so that it can exert its cytotoxic effect. Immunotoxin technology is well known to the art, and suitable toxic molecules include, without limitation, chemotherapeutic drugs such as vindesine, antifolates, e.g. methotrexate, cisplatin, mitomycin, anthrocyclines such as daunomycin, daunorubicin or adriamycin, and cytotoxic proteins such as ribosome inactivating proteins (e.g., diphtheria toxin, pokeweed antiviral protein, abrin, ricin, pseudomonas exotoxin A or their recombinant derivatives. See, generally, e.g., Olsnes and Pihl (1982) Pharmac. Ther. 25:355-381 and Monoclonal Antibodies for Cancer Detection and Therapy, Eds. Baldwin and Byers, pp. 159-179, Academic Press, 1985.

High affinity TCR variable regions specific for a particular superantigen are useful in treating animals and mammals, including humans believed to be suffering from a disease associated with the particular superantigen.

The high affinity TCR variable region compositions can be formulated by any of the means known in the art. They can be typically prepared as injectables, especially for intravenous, intraperitoneal or synovial administration (with the route determined by the particular disease) or as formulations for intranasal or oral administration, either as liquid solutions or suspensions. Solid forms suitable for solution in, or suspension in, liquid prior to injection or other administration may also be prepared. The preparation may also, for example, be emulsified, or the protein(s)/peptide(s) encapsulated in liposomes.

The active ingredients are often mixed with optional pharmaceutical additives such as excipients or carriers which are pharmaceutically acceptable and compatible with the active ingredient. Suitable excipients include but are not limited to water, saline, dextrose, glycerol, ethanol, or the like and combinations thereof. The concentration of the TCR variable region in injectable, aerosol or nasal formulations is usually in the range of 0.05 to 5 mg/ml. The selection of the particular effective dosages is known and performed without undue experimentation by one of ordinary skill in the art. Similar dosages can be administered to other mucosal surfaces.

In addition, if desired, vaccines may contain minor amounts of pharmaceutical additives such as auxiliary substances such as wetting or emulsifying agents, pH buffering agents, and/or adjuvants which enhance the effectiveness of the vaccine. Examples of adjuvants which may be effective include but are not limited to: aluminum hydroxide; N-acetyl-muramyl-L-threonyl-D-isoglutamine (thr-MDP); N-acetyl-nor-muramyl-L-alanyl-D-isoglutamine (CGP 11637, referred to as nor-MDP); N-acetylmuramyl-L-alanyl-D-isoglutaminyl-L-alanine-2-(1′-2′-dipalmitoyl-sn-glycero-3hydroxyphosphoryloxy)-ethylamine (CGP 19835A, referred to as MTP-PE); and RIBI, which contains three components extracted from bacteria: monophosphoryl lipid A, trehalose dimycolate and cell wall skeleton (MPL+TDM+CWS) in a 2% squalene/Tween 80 emulsion. Such additional formulations and modes of administration as are known in the art may also be used.

The TCR variable regions of the present invention and/or binding fragments having primary structure similar (more than 90% identity) to the TCR variable regions and which maintain the high affinity for the superantigen may be formulated into vaccines as neutral or salt forms. Pharmaceutically acceptable salts include but are not limited to the acid addition salts (formed with free amino groups of the peptide) which are formed with inorganic acids, e.g., hydrochloric acid or phosphoric acids; and organic acids, e.g., acetic, oxalic, tartaric, or maleic acid. Salts formed with the free carboxyl groups may also be derived from inorganic bases, e.g., sodium, potassium, ammonium, calcium, or ferric hydroxides, and organic bases, e.g., isopropylamine, trimethylamine, 2-ethylamino-ethanol, histidine, and procaine.

TCR variable regions for therapeutic use are administered in a manner compatible with the dosage formulation, and in such amount and manner as are prophylactically and/or therapeutically effective, according to what is known to the art. The quantity to be administered, which is generally in the range of about 100 to 20,000 μg of protein per dose, more generally in the range of about 1000 to 10,000 μg of protein per dose. Similar compositions can be administered in similar ways using labeled TCR variable regions for use in imaging, for example, to detect cells to which a superantigen is bound. Precise amounts of the active ingredient required to be administered may depend on the judgment of the physician or veterinarian and may be peculiar to each individual, but such a determination is within the skill of such a practitioner.

The vaccine or other immunogenic composition may be given in a single dose; two dose schedule, for example two to eight weeks apart; or a multiple dose schedule. A multiple dose schedule is one in which a primary course of vaccination may include 1 to 10 or more separate doses, followed by other doses administered at subsequent time intervals as required to maintain and/or reinforce the immune response, e.g., at 1 to 4 months for a second dose, and if needed, a subsequent dose(s) after several months. Humans (or other animals) immunized with the retrovirus-like particles of the present invention are protected from infection by the cognate retrovirus.

Standard techniques for cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described in Sambrook et al. (1989) Molecular Cloning, Second Edition, Cold Spring Harbor Laboratory, Plainview, N.Y.; Maniatis et al. (1982) Molecular Cloning, Cold Spring Harbor Laboratory, Plainview, N.Y.; Wu (ed.) (1993) Meth. Enzymol. 218, Part I; Wu (ed.) (1979) Meth Enzymol. 68; Wu et al. (eds.) (1983) Meth. Enzymol. 100 and 101; Grossman and Moldave (eds.) Meth. Enzymol. 65; Miller (ed.) (1972) Experiments in Molecular Genetics, Cold Spring Harbor Laboratory, Cold Spring Harbor, N.Y.; Old and Primrose (1981) Principles of Gene Manipulation, University of California Press, Berkeley; Schleif and Wensink (1982) Practical Methods in Molecular Biology; Glover (ed.) (1985) DNA Cloning Vol. I and II, IRL Press, Oxford, UK; Hames and Higgins (eds.) (1985) Nucleic Acid Hybridization, IRL Press, Oxford, UK; and Setlow and Hollaender (1979) Genetic Engineering: Principles and Methods, Vols. 1-4, Plenum Press, New York. Abbreviations and nomenclature, where employed, are deemed standard in the field and commonly used in professional journals such as those cited herein.

### Example 1

### Engineering T Cell Receptors for High Affinity Binding to TSST-1

TSST-1 interacts almost exclusively with the human Vβ2.1 (hVβ2.1) region and a significant fraction of patients with TSS exhibit expansions of T cells with hVβ2.1. The structure of hVβ2.1 in complex with SpeC showed that hVβ2.1 uses a greater number of hypervariable regions for contact, compared to the interaction of mouse Vβ8.2 with its three different SAg ligands. Thus, residues from all three complementarity determining regions (CDRs) and hypervariable loop 4 (HV4) contributed contacts with SpeC and the interface exhibited a greater buried surface area than mVβ8.2-SAg interfaces. While the structure of the hVβ2.1-TSST-1 complex has not been solved, a recent alanine mutagenesis study of TSST-1 revealed the key residues of TSST-1 that are involved in the interaction.

Yeast display techniques were used to engineer the TCR for higher affinity binding to the desired superantigen. These yeast display techniques are described in U.S. Pat. Nos. 6,759,243; 6,696,251; 6,423,538; 6,300,065; 6,699,658, which are incorporated by reference to the extent not inconsistent with the disclosure herewith.

Previous studies showed that single chain TCRs (Vβ-linker-Vα) or Vβ domains required mutations to display the properly folded proteins, as a fusion to the agglutinin receptor Aga2, on the surface of yeast. Subsequent studies showed that mutations that enabled surface display also yielded thermally stabilized, soluble V region domains that could be secreted from yeast or refolded from Escherichia coli inclusion bodies (data not shown).

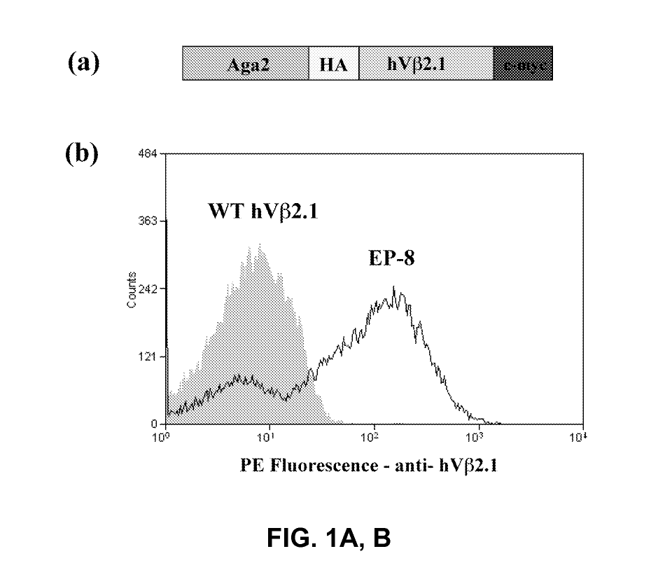

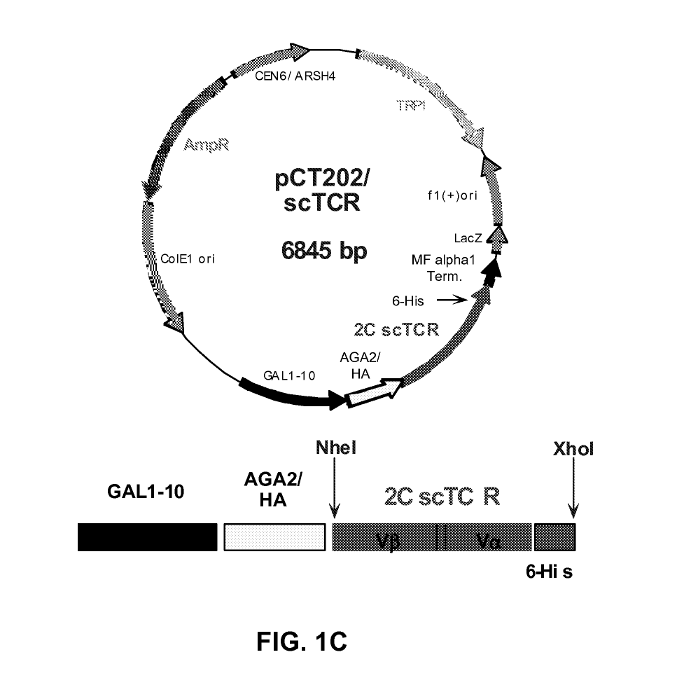

A fusion of genes encoding Aga2, a hemagglutinin (HA) epitope tag, hVβ2.1, and a c-myc epitope tag (FIG. 1(a)) was cloned into the yeast display vector shown in FIG. 1C. As anticipated, the wild-type hVβ2.1 region was not detected on the surface, as probed with a monoclonal antibody to this specific V region (FIG. 1(b)) or with an antibody to the C-terminal c-myc tag (data not shown). To identify a mutated hVβ2.1 domain variant that could be displayed on the yeast surface and that would allow expression in E. coli, the hVβ2.1 insert was subjected to error-prone PCR at an error rate resulting in an average of two mutations per Vβ molecule. PCR products were cloned by homologous recombination into the yeast display plasmid resulting in a library size of approximately 15×10⁶ transformants. The library was selected by fluorescence-activated cell sorting (FACS) with an anti-hV β2 antibody through three rounds and an anti-c-myc antibody for one round. After the final round of selection, yeast cells were plated and individual clones were screened for binding to the hVβ2-specific antibody. FIG. 1(b) shows an example of the improvement in surface display for one of the clones, EP-8.

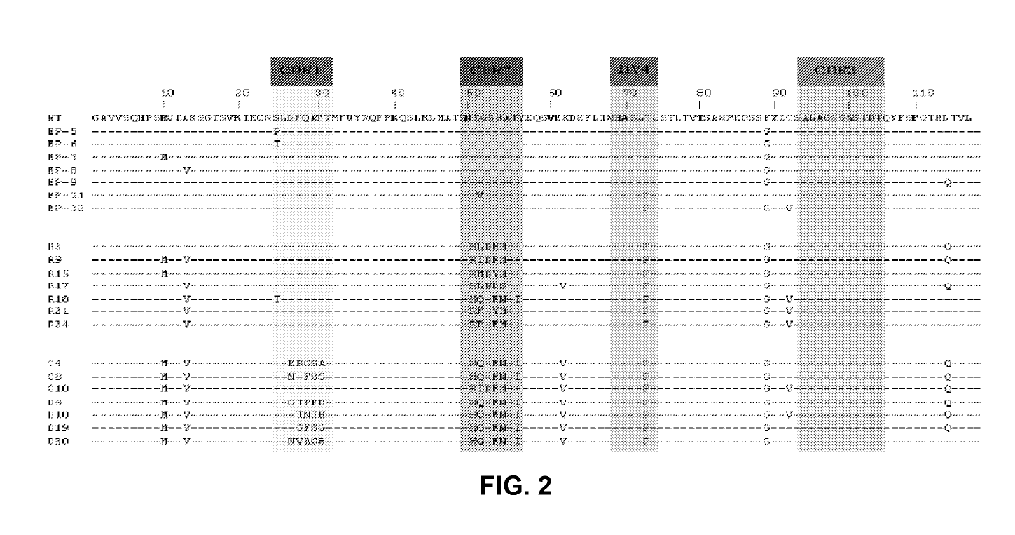

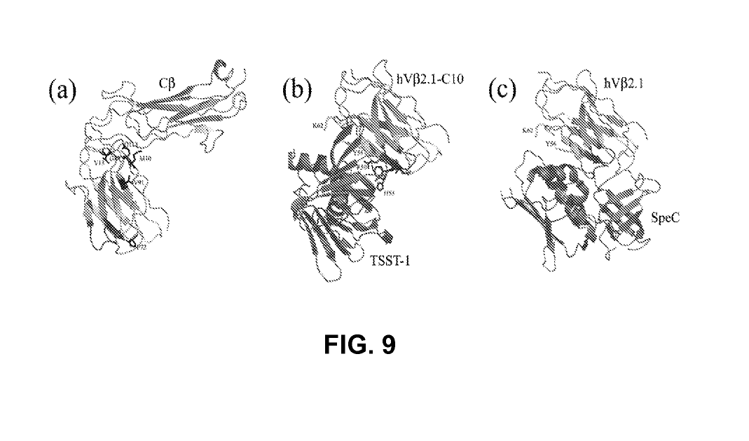

Ten clones (designated EP-series) with the highest levels of surface hVβ2.1, as judged with an anti-hVβ2 antibody, were chosen for sequence analysis. Seven unique sequences, with two or three mutations each, were identified (FIG. 2). Two of the mutations were present in the CDRs, one was present in HV4, and five mutations were present in FR regions. One of the FR mutations in particular, S88G, was the most prevalent of the mutations isolated, as it was found in six of the seven unique clones. Five of these mutations (R10M, A13V, L72P, S88G and R113Q) accumulated in almost every clone isolated after subsequent rounds of affinity maturation (see below), suggesting that these mutations were each important in stabilization and display. Four of the mutations are located at the Vβ surface, where the constant region (Cβ) would be located in the wild-type T cell receptor (FIG. 9(a), below). In full length β chains, this area is buried at the Vβ:Cβ interface and thus these mutations may act to prevent aggregation of the Vβ domain. The other mutation, L72P, is located at the other end of the Vβ region within the HV4 loop. The proline substitution may act to stabilize the local region surrounding this loop.

Isolation and Characterization of First Generation High Affinity hVβ2.1 Mutants

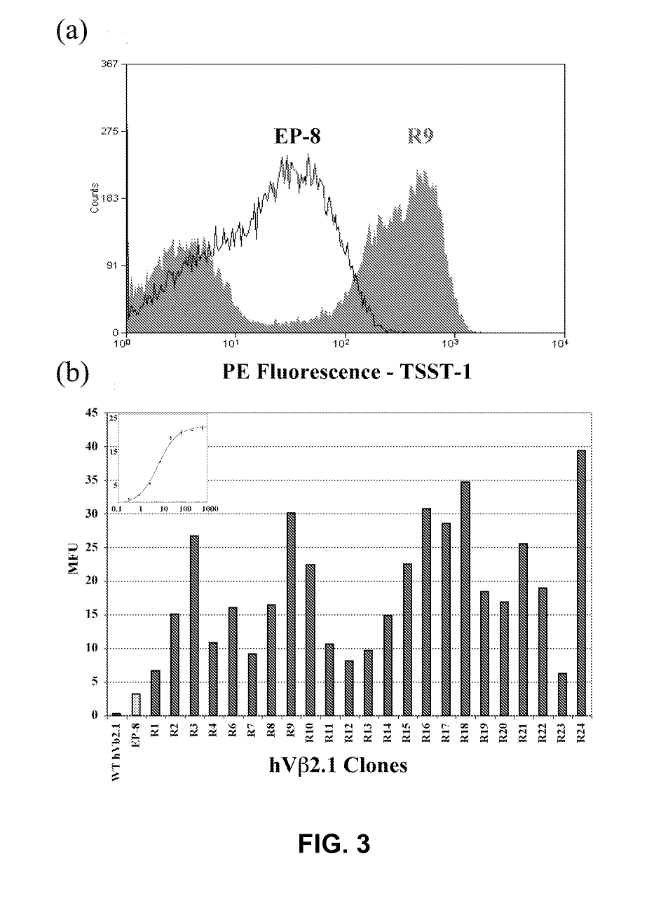

The low affinity of hVβ2.1 for TSST-1 (K_{D}=2.3 μM) prohibits the effective use of the soluble Vβ receptor as an antagonist for TSST-1-mediated toxicity. To engineer higher affinity mutants, the stabilized Vβ genes (EP-series, see above) were used as a starting point for affinity maturation. Because there is no crystal structure of the hVβ2.1-TSST-1 complex, the positions for site-directed mutagenesis were based on the structures of other Vβ-SAg complexes. As CDR2 is uniformly involved in contacts with SAgs, including the interaction between hVβ2.1 and SpeC, this region was chosen for the first round of affinity maturation. Five residues in this loop (50, 51, 52, 52a, and 53) were mutated randomly using degenerate oligonucleotides in splice extension PCR reactions with equal amounts of six unique stabilized clones isolated from the error-prone library as templates (see FIG. 2). The library of approximately 14×10⁶ independent clones was sorted through four cycles using decreasing concentrations of TSST-1. The first and second sorts were performed at a TSST-1 concentration of 1.8 μM (approximately equivalent to the K_{D} value of the wild-type hVβ2.1-TSST-1 interaction), the third sort was performed at 900 nM TSST-1, and the fourth sort was performed at 90 nM TSST-1 (approximately 20-fold below the K_{D} value). Twenty-four clones (designated R-series) were analyzed by flow cytometry for their ability to bind to 200 nM TSST-1, approximately tenfold below the K_{D} value of the wild-type. At this TSST-1 concentration, the stabilized clone EP-8 is weakly positive (a slight shift of the flow histogram) and the affinity matured clone R9 was strongly positive (FIG. 3(a)). Using the mean fluorescence units of each histogram, each of the 24 clones was compared to the stabilized clone EP-8 and all clones demonstrated an improvement over the stabilized mutant (FIG. 3(b)), which has an affinity similar to the wild-type hVβ2.1, as measured by surface Plasmon resonance (SPR) analysis (see below and Table 1). Fifteen clones isolated from the first affinity maturation library were sequenced to examine the mutations in the CDR2 loop (FIG. 2, and data not shown). Each of the clones sequenced was unique and contained a sequence that differed from the wild-type CDR2 of hVβ2.1. While there did not appear to be a strict consensus of any of the residues, there were strong preferences for either histidine or arginine at position 50 (from asparagine) and a histidine at position 53 (from lysine). There were also preferences for either aspartic acid or the wild-type glycine at position 52 and an aromatic residue at position 52a. Retention of the wild-type glycine at position 52 in many clones suggests that this residue may contribute the flexibility required for positioning other residues in this loop. While CDR2 of the wild-type hVβ2.1 contains two potentially charged residues (Glu51 and Lys53) and a net neutral charge, most of the mutated CDR2 regions were highly charged with a net positive charge. The exception was clone R17 that retained a net neutral charge (see below). The preference for an aromatic residue at position 52a may also indicate a hydrophobic interaction facilitates binding to TSST-1.

TABLE 1
Kinetic and affinity parameters for the interactions
between hVβ2.1 variants and TSST-1

| | kₐ (M⁻¹s⁻¹) | k_{d} (s⁻¹) | K_{A} (M⁻¹) | K_{D} (M) |
|---|---|---|---|---|
| EP-8^{a} | n.d.^{b} | n.d. | 1.67 × 10⁸ | 5.99 × 10⁻⁷ |
| R9 | 1.48 (±0.01) × 10⁴ | 1.86 (±0.02) × 10⁻³ | 7.95 × 10⁷ | 1.26 × 10⁻⁸ |
| C10 (Y56A) | 5.87 (±0.05) × 10⁴ | 3.99 (±0.02) × 10⁻³ | 1.47 × 10⁷ | 6.78 × 10⁻⁸ |
| C10 (K62A) | 2.14 (±0.01) × 10⁵ | 3.24 (±0.02) × 10⁻⁴ | 6.60 × 10⁸ | 1.52 × 10⁻⁹ |
| C10 | 3.28 (±0.01) × 10⁶ | 1.12 (±0.02) × 10⁻⁴ | 2.94 × 10⁹ | 3.41 × 10⁻¹⁰ |
| D10 | 2.56 (±0.01) × 10³ | 4.59 (±0.02) × 10⁻³ | 5.58 × 10⁸ | 1.79 × 10⁻²⁰ |

Measured by surface plasmon resonance with TSST-1 immobilized on chips.
^{a}Affinity parameters determined by equilibrium binding studies
^{b}n.d. not determined.

Isolation and Characterization of Second Generation High Affinity hVβ2.1 Mutants

Titration of the yeast-displayed mutant R9 with various concentrations of TSST-1 yielded an estimated binding affinity of 6 nM (FIG. 3(b), inset). To generate hVβ2.1 mutants with sub-nanomolar affinity, three of the first generation mutants (R9, R17 and R18) were used as templates for the generation of additional mutated libraries. These clones were selected on the basis of their high affinity binding to TSST-1, as well as having distinctly different CDR2 sequences (FIG. 2). As hVβ2.1 binding to SpeC involves contacts with HV4 and CDR1, it was reasoned that hVβ2.1 might also use these regions for binding to TSST-1. Thus, separate libraries in CDR1 (residues 27, 27a, 28, 29, 30), HV4 (residues 68, 69, 70, 71, 72), and an additional library in the CDR2 loop (residues 52a, 53, 54, 55, 56) were generated to extend the range of residues that were mutated. These three libraries were pooled in equal ratios for flow cytometric sorting.

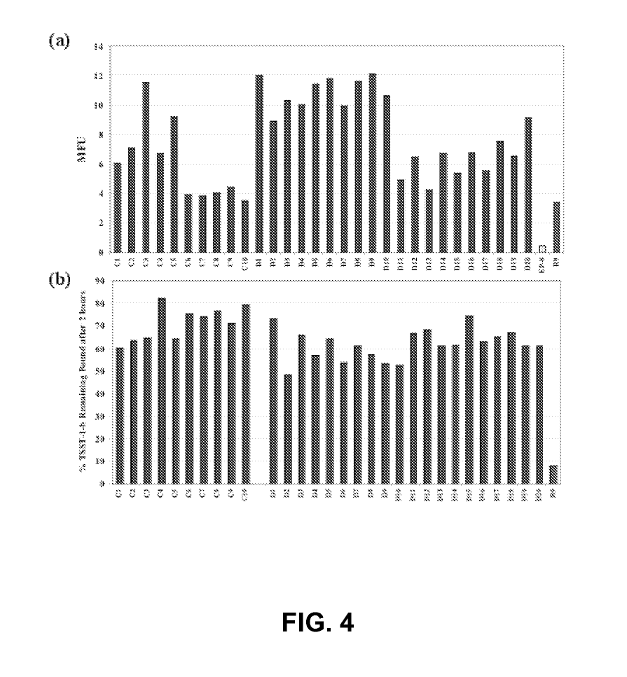

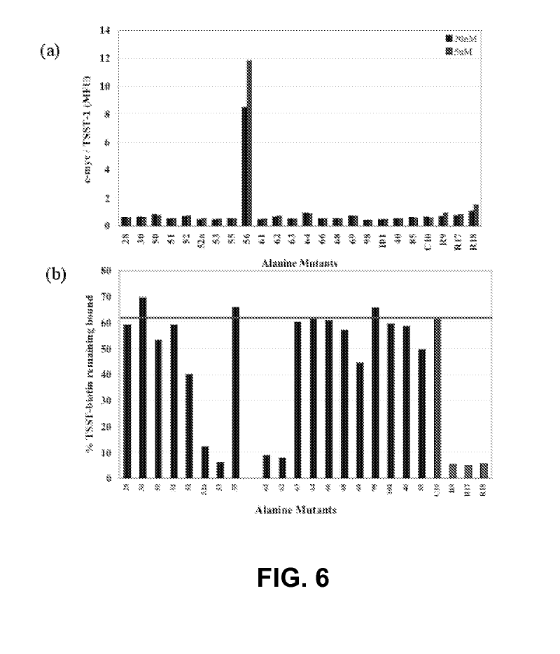

Because the initial flow cytometry experiment determined the K_{D} value of hVβ2.1 mutant R9 to be about 6 nM, a selection strategy other than an equilibrium-based methodology was required because at this concentration, the number of surface displayed receptors, in a library of 10⁶ yeast cells, begins to exceed the number of ligand molecules (in a 1 to 2 ml sample). Thus, an off-rate based screening strategy was adopted in an effort to isolate mutants with improved affinity exceeding that of the first-generation clones (R-series). The off-rate method involves incubation of yeast cells with labeled TSST-1 under equilibrium conditions, followed by washing and incubation with a tenfold molar excess of unlabeled TSST-1. Pilot analysis of clone R9 showed that less than 10% of biotin-TSST-1 remained bound after two hours under these conditions (FIG. 4(b), and data not shown). Thus, yeast libraries were sorted after the two-hour dissociation period, and clones were isolated after the third (designated C-series) and fourth (designated D-series) cycles of selection. A total of 30 clones were examined for their ability to bind 5 nM TSST-1 in comparison to clones EP-8, R9, R17, and R18 (FIG. 4(a)). Each clone exhibited binding that was equal to or better than the R-series of clones at this TSST-1 concentration. When the clones were examined using a single time-point off-rate analysis, similar to that used for sorting the library, it was evident that every clone showed significant improvements when compared to the first generation R-series clones (FIGS. 4(b) and 6(b), below). For example, clones R9, R17, and R18 had less than 10% of the labeled TSST-1 remaining bound after 2 h at 25° C., while the off-rate selected clones had 50% or more of the labeled TSST-1 remaining bound.

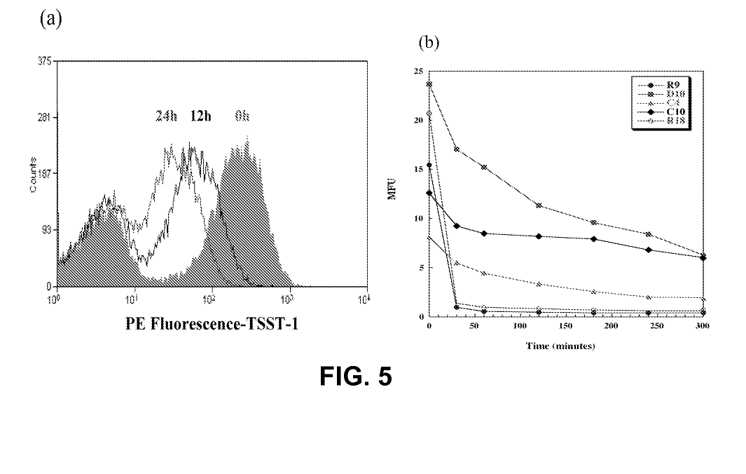

To further examine TSST-1 off-rates, various mutants were examined for binding to labeled TSST-1 over a 5 h time period and at a higher temperature (37° C.). Histograms of clone C10 at several time points are shown in FIG. 5(a). Mean fluorescence units (MFU) from histograms at various time points were plotted for each of the analyzed clones (FIG. 5(b)). While bound biotinylated TSST-1 from the two first-generation clones (R9, R18) was reduced to background levels by the first time point (30 min), three clones (C4, C10 and D10) retained significant levels of bound biotinylated TSST-1 even at the end of the time course (5 h). Clone 10 retained approximately 50% of the TSST-1 after 5 h at 37° C. This time course was taken out to 24 h at 37° C., and while the levels of bound TSST-1 decreased, about 15% of TSST-1 remained bound to C10 after 24 h (FIG. 5(a)).

Fourteen clones isolated from the off-rate based selection were sequenced (FIG. 2, and data not shown). Eleven clones contained CDR1 mutations derived from the CDR1 library. Three clones did not contain mutations in the regions of the second generation libraries (CDR1, CDR2 extended, and HV4), but they contained single-site mutations (e.g. clone C10). None of the clones were derived from the second extended CDR2 library, suggesting that residues 54 to 56 were critical for TSST-1 binding and their sequences could not be optimized. Each clone contained the FR3 mutation E61V, which was presumably incorporated from clone R17 through a PCR-derived error. As described earlier, each of the affinity-matured clones also contained the stabilizing mutations that may act additively in the enhanced surface display of the hVβ2.1 region, as has been observed for mutations in the 2C TCR.

The absence of preferred mutations in the CDR1 clones, and the fact that clone C10 lacked CDR1 mutations altogether, suggests that CDR1 may not be involved in a significant way in binding TSST-1. Detailed inspection of the sequences indicates that the longer off-rates of these clones appear to be due to residues in the CDR2 and/or the E61V mutation, or a combination of these mutations. These results are supported by mutagenesis results described below.

Alanine Scanning Mutagenesis of a High-Affinity hVβ2.1 Mutant C10

To further examine the role that the individual Vβ residues play in the interactions with TSST-1, alanine mutagenesis of selected wild-type and mutated residues of hVβ2.1 mutant C10 was performed. C10 was chosen as it exhibited high affinity with a decreased off-rate and yet contained the fewest number of mutations. Residues were chosen in part based on contact residues within the hVβ2.1-SpeC complex and also to define the mechanism by which C10 achieves high affinity. C10 alanine mutants were constructed in the yeast display vector in order to allow rapid analysis of binding without the need for protein purification. Similar approaches have been used to examine the role of individual residues or to map the binding epitopes of monoclonal antibodies. Mutants were first tested for their levels of surface expression with the anti-c-myc antibody to determine if mutation to alanine affected the folding and stability of the protein. All mutants expressed detectable c-myc epitopes, with levels that were similar to or slightly improved relative to C10 Vβ (data not shown). To quantify the binding to TSST-1, each mutant was analyzed for binding to 5 and 20 nM TSST-1 and a ratio of anti-c-myc to TSST-1 binding was determined (FIG. 6(a)). These concentrations of TSST-1 are about 12 and 50-fold above the estimated K_{D} of C10, respectively (see below), and thus were used to detect significant changes in affinity. Under these conditions, only Y56A, a mutation of a wild-type residue, was shown to affect significantly the binding of TSST-1. Further binding analysis by flow cytometry at higher TSST-1 concentrations and by SPR with soluble Y56A protein showed that TSST-1 binding affinity was reduced by ˜100-fold (see below).

In order to characterize more precisely the impact of each mutation, a single point off-rate analysis of the yeast-displayed mutants was performed. Mutants were incubated with 5 nM biotinylated TSST-1, followed by incubation with a 50-fold molar excess of unlabeled TSST-1 for 2 h at 37° C. (FIG. 6(b)). Time points were taken at time zero (before unlabeled ligand was added) and at 2 h to calculate the percent of remaining bound ligand. Based on this study, four alanine mutations were shown to affect the off-rate significantly: F52aA, H53A, V61A, and K62A. Mutation of these residues to alanine reduced the fraction of bound ligand to levels comparable to that of clone R9. Because two of the four residues are present in R9 (F52a and H53), it was predicted that residues 61 (valine) and 62 (lysine) contribute to the longer off-rate of clone C10.

It is worth noting that one clone, R17, from the first-generation of high-affinity mutants contained the E61V mutation, yet did not exhibit the slow off-rate characteristic of C10 (FIG. 6(b)). The only notable sequence difference between R17 and other R-series mutants is that the net charge of the CDR2 was neutral, rather than positive. Since the H53A mutation, like the E61V mutation, appears to affect the off-rate of C10, it is believed that two regions of electrostatic interactions are necessary to achieve the slow off-rate and high-affinity of C10. These regions include CDR2 and FR3.

### Binding Analyses of Selected Mutants by Surface Plasmon Resonance

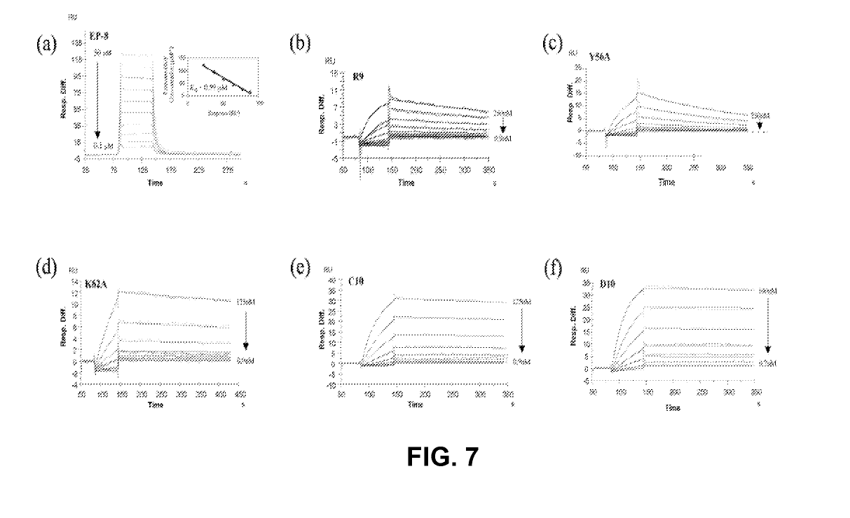

Several of the clones from the affinity maturation process, and single-site mutants, were expressed in E. coli, refolded from inclusion bodies, and examined using surface plasmon resonance (SPR) to measure the affinity and kinetics of their interactions with TSST-1 (FIG. 7 and Table 1). As the wild-type hVβ2.1 domain does not express well in E. coli (data not shown), the stabilizing mutations appear to enable expression and refolding. The stabilized mutant EP-8 exhibited an affinity of 0.6 μM, similar to that observed previously for the full length wild-type hVβ2.1 (VβCb) (K_{D}=2.3 μM). Based on SPR results with the higher affinity mutants, findings from the flow cytometric analysis on yeast were confirmed. The affinity of the first generation mutant R9 was increased by 180-fold (K_{D}=12.6 nM), compared to the affinity of wild-type hVβ2.1. The affinity of the second generation mutants C10 and D10 were increased by 6800 and 12,800-fold (K_{D}=340 pM and 180 pM, respectively). The 180-fold higher affinity of R9 was accomplished through substitutions of CDR2 residues (residues 50-53: wildtype, NEGSK; R9, RIDFH). As indicated above, the highly charged nature of each of the CDR2 mutants may suggest that electrostatics play a role in this affinity increase. Alternatively, enhanced affinity could be achieved through an increase in the buried hydrophobic surface area. Analyses of the binding kinetics indicate that the affinity increases from R9 to C10 and D10 are due to significantly reduced off-rates (17 and 41-fold) and only modest enhancements of on-rates (2.2 and 1.7-fold). The 17-fold slower off-rate of C10 compared to R9 is consistent with the results derived from flow cytometry experiments with C10.

C10 differs from R9 at only two residues, E61V and 191V. The faster off-rate observed in the V61A mutant, and the observation that all of the second-generation clones contain the E61V mutation, suggest that this mutation accounts for the slower off-rate and corresponding increase in affinity. This effect could be due to the loss of an acidic side-chain at position 61, enabling a productive electrostatic interaction between TSST-1 and the lysine at position 62 in hVβ2.1. In support of this possibility, the K62A mutation also resulted in a significant reduction in the off-rate. In addition, soluble C10-K62A exhibited an affinity only twice that of R9, 17-fold reduced compared to C10 (Table 1). The lower affinity was due to a sevenfold faster off-rate and a 2.6-fold slower on-rate. Because the lysine side-chain is known to contribute to the overall hydrophobicity, the K62A mutation may act through a reduction in buried hydrophobic surface area. Whatever the mechanism is, these results show the involvement of the FR3 region at positions 61 and 62 in formation and stability of the C10-TSST-1 complex.

Because Tyr56 of hVβ2.1 appeared to be the most important of the residues tested for binding to TSST-1, the binding properties of the soluble C10-Y56A mutant were examined (Table 1). The binding affinity of C10-Y56A was reduced 200-fold (K_{D}=147 nM), with a 35-fold faster off-rate and a six-fold slower on-rate. While these results are based on the affinity of the engineered hVβ2.1 variant C10, the fact that Y56 is in the wild-type protein and that this residue represents one of the few unique residues of hVβ2.1 compared to other human Vβ regions, suggest that it contributes significantly to the binding energy and specificity of hVβ2.1 for TSST-1.

TSST-1 and SpeC Binding to Overlapping Epitopes on hVβ2.1

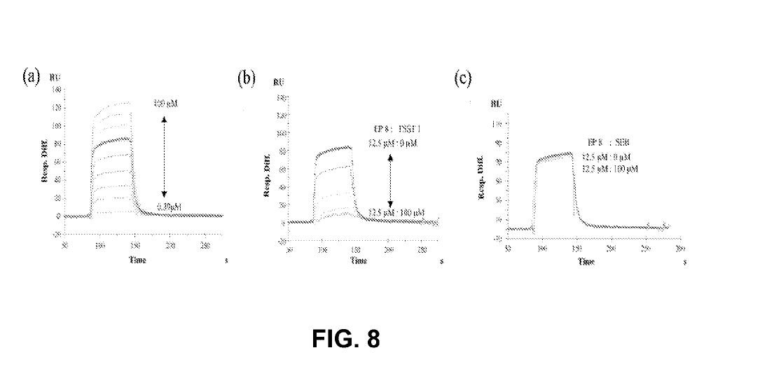

To determine whether there is overlap in the binding sites for TSST-1 and SpeC on hVβ2.1, a competition experiment was performed. In this experiment, SpeC was immobilized on the SPR chip, the engineered hVβ2.1 called EP-8 was injected at various concentrations, and an affinity of approximately 6 μM was measured (FIG. 8(a)). To determine if TSST-1 competed for binding of the hVβ2.1 (EP-8), various concentrations of TSST-1 were mixed with 12.5 μM EP-8, FIG. 8(a) and (b)). The ability of EP-8 to bind the SpeC was reduced as more TSST-1 was present in the mixture (FIG. 8(b)), as would be expected if there were competition for the same binding site. In a control experiment, the SAg SEB, which does not bind to hVβ2.1, was used at the same concentrations as TSST-1 and competition was not observed (FIG. 8(c)).

### Discussion

Secreted bacterial toxins such as TSST-1 act as SAgs by stimulating cytokine release from a large fraction of T lymphocytes. The elevated systemic cytokine levels can lead to toxic shock syndrome and ultimately multi-organ failure. The mechanism of action of bacterial SAgs is now well known and a number of SAgs have been examined for the molecular basis by which they interact with T cells. However, the molecular details of the interaction of TSST-1 with hVβ2.1 has so far been refractory to structural analyses. TSST-1 is particularly important clinically, as it represents one of the most common toxins involved in TSS and as such it has significant involvement in staphylococcal mediated diseases. hVβ2.1, the specific major target associated with the effects of TSST-1 in humans was studied by: (1) engineering a stabilized hVβ2.1 domain that would be amenable to expression in E. coli and directed evolution by yeast display; (2) identifying specific targeted mutations in hVβ2.1 that would increase the affinity of the hVβ2.1-TSST-1 interaction; and (3) generating and analyzing selected single-site mutations of hVβ2.1 that would reveal both the mechanisms by which higher affinity was achieved and the possible docking orientation of the hVβ2.1-TSST-1 complex.

The engineering of a stabilized, surface displayed hVβ2.1 mutant enabled expression of the protein in E. coli and subsequent refolding to concentrations sufficient for biochemical analyses. The mutations reside largely at the Vβ face, which would normally be buried at the interface with the Cβ region (FIG. 9(a)). The stabilized hVβ2.1 mutant EP-8 bound to TSST-1 and SpeC with affinities that are close to those measured for the full-length β-chain.

These results also implicate the region spanning the CDR2 loop and FR3 of hVβ2.1 (including residues 51-54, 56 and 61-62) as energetically critical for TSST-1 binding. Using the hVβ2.1 as a starting point, an energy minimized model of the high affinity mutant C10 was generated (the C10 mutations are solvent exposed). A hypothetical model of the hVβ2.1-TSST-1 complex (FIG. 9(b)), was generated by manually docking the TSST-1 in a position on hVβ2.1-C10 that is consistent with each of the following observations (described in more detail below): (1) The most important energetic residue (Tyrβ56) lies near the center of the complex; (2) the framework region 61 to 63, which was shown to be important in the engineered hVβ2.1 mutants, is in contact with a region of TSST-1 that shows electrostatic complementarity; (3) the CDR3 of hVβ2.1 which was not involved in binding based on mutational analyses, is not in contact with TSST-1; (4) TSST-1 residues that were previously identified to be important in hVβ2.1 binding are located within contact distance in the hypothetical model; and (5) the position of TSST-1 on the hVβ2.1 region overlaps that of the binding site for SpeC (FIG. 9(c)) and thus SpeC binding would be competed by TSST-1.

In this model, both the positive charges on CDR2 (Arg50 and His53) and FR3 (Lys62) are positioned near negatively charged residues (e.g. Asp11 and Asp18) in TSST-1. Alternatively, it is possible that mutations such as S52aF and E61Vact by increasing the buried hydrophobic surface area at the hVβ2.1-TSST-1 interface. Consistent with this possibility, the F52aA and V61A mutations both reduced the affinity, perhaps as a consequence of the reduced hydrophobicity of an alanine side-chain compared to phenylalanine and valine side-chains. Tyrosine 56 is predicted to be at the center of the interface, in a key position to interact with TSST-1. This location is consistent with the significant energetic contribution of Tyr56 (i.e. 100-fold decrease in binding for the Y56A mutant). Recent studies with high-affinity mouse Vβ mutants also showed that energetically important residues were located at the center of the interface. Thus, it is reasonable to predict that Tyr56 is located at the center of the engineered hVβ2.1-TSST-1 interaction. The identification of Tyr56 as an important residue within hVβ2.1 is also consistent with the observation that this residue is nearly unique among over 50 known human Vβ regions. In fact, only human Vβ4 contains a tyrosine at this position, but Vβ4 lacks positive charges in the CDR2 or at position 62 in FR3. This may explain why TSST-1 appears to be unusual among SAgs in that it is known to stimulate only a single human Vβ region, hVβ2.1. In further support of the role of Tyr56, mouse T cells that bear mouse Vβ15 are expanded by stimulation with TSST-1 and mouse Vβ15 contains a tyrosine at position 56.

On the other hand, the putative electrostatic interactions or increased buried hydrobicity involved in the hVβ2.1-C10 interaction appear to be at least in part a consequence of engineering the CDR2 and FR3 regions to enhance these effects. Interestingly, the wild-type hVβ2.1 is highly charged at these positions and while the electrostatic effects may be masked to some degree by nearby neutralizing residues (e.g. Glu51 and Glu61), it is possible that there are electrostatic contributions that facilitate the docking of the wild-type hVβ2.1 in an orientation similar to that predicted for hVβ2.1-C10. Sequence analysis of human Vβ regions shows that the combination of lysine residues at positions 53 and 62 are unique to hVβ2.1. While two Vβ regions (Vβ19, Vβ30) have a lysine at position 62, they lack a positive charge within the CDR2. Furthermore, many Vβ regions actually contain aspartic acid or glutamic acid residues at position 53 or 62, which could be detrimental to productive electrostatic interactions with TSST-1, based on the model. While structural studies will be required to examine these issues, the model suggests a different three-dimensional orientation of TSST-1 on hVβ2.1 compared to the hVβ2.1-SpeC complex (FIG. 9(c)). In this model, TSST-1 does not extend to the CDR3 of hVβ2.1, and it is shifted further toward the FR3 region. While the hVβ2.1 footprints of the TSST-1 and SpeC contact regions may differ, the model predicts that TSST-1 and SpeC have overlapping binding regions on hVβ2.1 in the area of CDR2.

### Neutralizing Agents for TSST-1

The engineering of soluble, high-affinity Vβ receptors for TSST-1 that have half-lives of many hours provides the basis for effective neutralizing agents against TSST-1. Soluble Vβ domains can be engineered with high affinity binding to superantigens. For example, soluble Vβ domains having ˜1500-fold higher binding affinity (K_{D}˜5 nM) for SAg Staphylococcal enterotoxin C3 (SEC3) have been engineered. Soluble forms of these Vβ mutants were effective inhibitors of the in vitro activity of SEC3. It is desirable to generate Vβ domains with even higher affinities, since the enterotoxins are toxic at very low concentrations. Thus, this new generation of hVβ2.1 mutants, with greater than 10.000-fold improvements in affinity above the wild-type interaction (K_{D} value of hVβ2.1-D10 of 180 pM affinity, for example), are useful as protein-based neutralizing agents against TSST-1.

### Materials and Methods

Cloning and yeast display of human Vβ2.1

The gene for human Vβ2.1, residues 1-117, containing the mutation C13A, was cloned into the yeast display vector, pCT302, as a NheI-BamHI fragment. This construct contains two epitope tags, HA on the N terminus, and two tandem c-myc tags on the C terminus that serve as internal controls for protein expression. To generate a library of random mutants, the hVβ2.1 gene was amplified from the pCT302 plasmid using flanking primers with a method of error-prone PCR to give a 0.5% error rate (data not shown). The PCR product was transformed along with NheI-BglII digested pCT302 into the yeast strain EBY100, which allows the PCR product to be inserted into the plasmid by homologous recombination. The resulting library of approximately 10⁶ transformants was grown on selective media for 48 h.

### Fluorescence Activated Cell Sorting (FACS)

The randomly mutated hVβ2.1 library was cultured for 36 h at 20° C. in medium containing galactose to induce protein expression. One hundred million cells were incubated with 10 μg/ml of mouse anti-human Vβ2 monoclonal antibody (Beckman Coulter). Cells were stained with a 1:50 dilution of goat F(ab′)2 anti-mouse Ig-RPE (Southern Biotech) and selected on a MoFlo high-speed cell sorter (Cytomation). The most fluorescent cells (1%) were collected, cultured overnight in selective media, and then induced in galactose-containing media for 20 h. For the second sort, about 50×10⁶ cells were incubated with a 1:50 dilution of anti c-myc (9E10) antibody (Roche), followed by a 1:50 PE-labeled secondary antibody. For the third sort, cells were incubated with a 1:20 dilution of the anti-human Vβ2 antibody (selecting the top 0.5%), and for the fourth sort cells were incubated with a 1:50 dilution of anti-human Vβ2 antibody (selecting the top 0.25%). After four rounds of sorting, individual clones were obtained by plating on selective media.

### Flow Cytometry of Isolated Mutants

Individual yeast clones were cultured in glucose-containing media at 30° C. and induced in galactose-containing media at 20° C. for 30 h. Expression levels of hVβ2.1 were examined by incubating 0.4×10⁶ yeast cells with anti-HA antibody (Covance) (1:75 dilution), anti-c-myc 9E10 antibody (1:75 dilution), or anti-human Vβ2 antibody (1:50 dilution) in PBS-BSA for one hour on ice. After washing, cells were incubated with PE conjugated secondary antibody (1:50 dilution). TSST-1 binding was measured by incubating cells with various concentrations of biotinylated TSST-1 (Toxin Technology), followed by streptavidin-PE (BD Pharmingen) at a 1:500 dilution. Fluorescence levels were measured using a Coulter Epics XL flow cytometer gating on a healthy yeast population.

Affinity Maturation of hVβ2.1

The genes encoding stabilized hVβ2.1 mutants were amplified using site-directed mutagenesis with overlapping degenerate primers (with NNS codons). Five residues in the CDR2 (50, 51, 52, 52a and 53) were randomized by this method. DNA from stabilized mutant clones EP-6, 7, 8, 9, 11, and 12 were pooled in equal amounts to use as the template DNA for the PCR. PCR products were incorporated into the yeast display plasmid pCT302 by homologous recombination to generate a library of 14×10⁶ independent transformants. The CDR2 library was sorted using a similar approach as described above, except that yeast cells were incubated with decreasing concentrations of biotinylated TSST-1 for each round of sorting, followed by a 1:1000 dilution of streptavidin-PE. Yeast cells were sorted through four cycles, and clones isolated from the fourth cycle were plated on selective media for further analysis. In a second round of affinity maturation, clones R9, R17, and R18 were used as templates. Libraries were constructed in CDR1 (residues 27, 27a, 28, 29 and 30), CDR2 (residues 52a, 53, 54, 55 and 56), and HV4 (residues 68, 69, 70, 71, and 72). To select for higher affinity mutants with increased off-rates, the three libraries were pooled in equal amounts, incubated with 5 nM biotinylated-TSST-1 for 1 h on ice, followed by an incubation with a tenfold molar excess of unlabeled TSST-1 for 2 h at 25° C. Yeast cells were selected using these conditions through four cycles of sorting, and clones from the third and fourth cycle were plated.

### Alanine Scanning Mutagenesis

Alanine residues were introduced into the human Vβ2.1 clone C10 in the following residues: CDR1 (Q28, T30), CDR2 (R50, I51, D52, F52a, H53, T55, Y56), FR3/HV4 (V61, K62, D63, K64, L66, N68, H69), and CDR3 (S98, S101). In addition two surface-exposed residues not predicted to be near the binding interface, K40 and E85, were included as controls. These single-site alanine mutations were constructed using the PCR method of splicing by overlap extension. The single-site mutants were transformed into yeast along with linearized pCT302 plasmid. Mutations were confirmed by sequencing and expression of the alanine mutants on the surface of the yeast was induced as described above. Alanine mutants were analyzed individually by flow cytometry.

### Surface Plasmon Resonance

Affinity-matured variants of hVβ2.1 were expressed in E. coli and refolded in vitro from inclusion bodies as described for murine Vβ8.2 variants affinity-matured for SEC3 binding. Affinity and kinetic analyses of the interactions between hVβ2.1 variants and TSST-1 were determined using a BIAcore 3000 SPR instrument (BIAcore) in 10 mM Hepes buffer containing 150 mM sodium chloride, 3.4 mM EDTA and 0.005% (v/v) surfactant P-20, at 25° C. TSST-1 at a concentration of 20 μg/ml in 10 mM sodium acetate (pH 4.6) was immobilized (˜250 resonance units) to a CM5 sensor chip (Biacore) using standard amine coupling methods. Staphylococcal entertoxin B (SEB) in an equivalent surface density was used as the control surface, as there is no specific binding between hVβ2.1 and SEB. All of the binding experiments were carried out at a flow rate of 25 μl/min. Pulses of 10 mM HCl were used to regenerate both surfaces between injections. SPR data for association (kₐ) and dissociation (k_{d}) rates, as well as the dissociation constant (K_{D}) were determined by globally fitting the data from different concentrations to a simple 1:1 Langmuir binding model using BIAevaluation software version 4.1. For the EP-8 variant, which exhibits kinetics that are not possible to measure accurately by SPR, the affinity (K_{D}) was determined by the Scatchard analysis of equilibrium binding of varying concentrations.

A competition assay to determine if TSST-1 and SpeC compete for binding of hVβ2.1 was performed using a CM5 sensorchip with SpeC (˜500 RU) immobilized via standard amine coupling. Serial dilutions of the stabilized hVβ2.1 mutant EP-8 (100 μM-0.39 μM) were injected over the SpeC surface. Non-linear regression analysis yielded an affinity of ˜6 μM for the EP-8-SpeC interaction (data not shown). For the competition experiment, mixtures of EP-8 and TSST-1 were injected over the SpeC surface. The concentration of EP-8 was held constant at 12.5 μM while the concentration of TSST-1 was varied from 100 μM to 12.5 μM. As a control, an identical experiment was performed, in which SEB, which does not bind to EP-8/hVβ2.1, was used.

### Molecular Modeling

A model of C10 hVβ2.1 was constructed using the coordinates of hVβ2.1 in complex with SpeC(PDB accession code 1 KTK). The C10 model was subjected to energy minimizations using the Gromos96 reaction field in Swiss PDB DeepView. Minimizations were performed using 50 steps of steepest descent and 50 steps of conjugate gradient. The model of the hVβ2.1-TSST-1 complex was based on the C10 energy minimized model and the crystal structure of TSST-1 (PDB accession code 2TSS). The molecules were docked manually using the program MacPyMOL† and all structural representations were prepared using MacPyMOL.

### Example 2

### Long-Range Cooperative Binding Effects in a T Cell Receptor Variable Domain

Interactions between proteins are essential for nearly all cellular processes (N. R. Gascoigne et al. (2004) Curr Opin Immunol 16:114-9; T. Pawson et al. (2000) Genes Dev 14:1027-47; A. J. Warren (2002) Curr Opin Struct Biol 12:107-14) and aberrant protein-protein interactions contribute to the pathogenesis of numerous human diseases (J. F. Rual et al. (2005) Nature 437:1173-8). As the genome-wide mapping of protein-protein interactions has identified many of the molecular components of numerous physiological and pathological processes (S. Li et al. (2004) Science 303:540-3; T. Bouwmeester et al. (2004) Nat Cell Biol 6:97-105; L. Giot et al. (2003) Science 302:1727-36; P. Uetz et al. (2000) Nature 403, 623-7; T. Ito et al. (2001) Proc Natl Acad Sci USA 98:4569-74) and structural genomics efforts have determined structures of many of the constituent protein domains involved in these interactions, the ability to predict the binding specificities and energies of protein complexes from protein structures alone has reached paramount importance. Although significant progress in developing computational methods for the quantitative predictions of protein-protein interactions has been made recently (R. Guerois et al. (2002) J Mol Biol 320:369-87; S. Huo et al. (2002) J Comput Chem 23:15-27; T. Kortemme et al. (2002) Proc Natl Acad Sci USA 99:14116-21; I. Massova et al. (1999) J. Am. Chem. Soc. 121:8133-8143; K. A. Sharp (1998) Proteins 33:39-48), the current robustness of these algorithms is not such that the laborious task of determining the structure of a given protein complex can be circumvented. It is clear that these methods are unable to account for aspects of molecular recognition that are important in determining complex formation, but for which there is currently a fundamental lack of understanding.

It has been known for some time that protein-protein interfaces are structural and energetic mosaics. Certain amino acid residues within an interface contribute significantly to the binding energy, and are thus termed “hot spots” (T. Clackson et al. (1995) Science 267:383-6; A. A. Bogan et al. (1998) J Mol Biol 280:1-9; W. L. DeLano (2002) Curr Opin Struct Biol 12:14-20), while other residues are energetically silent with respect to the interaction. These hot spots, furthermore, are not homogeneously distributed throughout the interface, but are instead clustered to form “hot regions” (O. Keskin et al. (2005) J Mol Biol 345:1281-94; D. Reichmann et al. (2005) Proc Natl Acad Sci USA 102:57-62). Further contributing to the heterogeneity of protein-protein interfaces is the frequent presence of cooperativity, such that the energetic contribution to binding of a protein that has been simultaneously mutated at multiple residues is significantly different than the summation of the changes in binding energy of the single-site mutants (S. Albeck et al. (2000) J Mol Biol 298:503-20; B. Bernat et al. (2004) Biochemistry 43:6076-84; J. Yang et al. (2003) J Biol Chem 278:50412-21).

The theory that residues within a single hot region are energetically cooperative, while those residing in distinct hot regions are strictly additive has arisen from both computational and experimental studies, including: a recent analysis (O. Keskin et al. (2005) J Mol Biol 345:1281-94) of a structurally non-redundant database of all hot regions (O. Keskin et al. (2004) Protein Sci 13:1043-55) currently in the Protein Data Bank (H. M. Berman et al. (2000) Nucleic Acids Res 28:235-42); and the mutational, energetic and structural analysis of residues within two hot regions of the TEM1-β-lactamase-β-lactamase inhibitor protein (TEM1-BLIP) complex (D. Reichmann et al. (2005) Proc Natl Acad Sci USA 102:57-62). If, in all protein complexes, cooperative energetics existed only within hot regions, and not between them, the quantitative prediction of protein-protein interactions may be considerably simplified.

A further implication of the notion that distal regions of a protein-protein interface do not cooperate is that long-range conformational effects (e.g. allosterism) are also uncommon. Thus, binding at one site on a protein is unlikely to influence binding of a different ligand at a distal site.

In order to test whether cooperativity could exist between hot regions, the interaction between affinity maturation variants of the human T cell receptor (TCR) variable domain 2.1 (hVβ2.1) and the superantigen (SAG) toxic shock syndrome toxin-1 (TSST-1) was analyzed. TSST-1 is the major causative agent of staphylococcal toxic shock syndrome (P. Schlievert et al. (1981) J Infect Dis 143:509-16: J. K. McCormick et al. (2001) Ann Rev Microbiol 55:77-104) and these hVβ2.1 variants were engineered as potential therapeutic agents for SAG-mediated disease (R. A. Buonpane et al. (2005) J Mol Biol 353:308-21). The highest affinity variant, D10, bound TSST-1 with an affinity of 180 pM, or 30,000-fold tighter than wild type hVβ2.1 (R. A. Buonpane et al. (2005) J Mol Biol 353:308-21). By mutating each of the D10 variant residues and individually measuring the affinities for TSST-1 of the subsequent single-site mutants by surface plasmon resonance (SPR) analysis, it is shown that four of the variant residues contribute significantly to affinity maturation. These residues are located in two distinct hot regions that are separated by about 22 Å. Mutations in these two distinct hot regions of hVβ2.1 are energetically cooperative when binding to TSST-1. Analysis of the hVβ2.1 structure (E. J. Sundberg et al. (2002) Structure 10:687-99) provides insight into how cooperativity between hot regions might occur in some protein-protein interactions but not in others. The protein core may act as an energetic sink to regulate inter-hot regional cooperative energetics. Furthermore, the TCR functions as a macromolecular complex with multiple CD3 subunits and the accessory molecules CD4 or CD8. Evidence for conformational effects between distant regions supports the view that inter-subunit associations are influenced by peptide-MHC (pMHC) binding. Accordingly, binding of pMHC by complementarity determining regions (CDRs) of the V domains could effect the association of other subunits, leading to enhanced signaling by the complex.

### Results and Discussion

### Identification of Energetically Significant Variant Residues in the Affinity Maturation Pathway

The high-affinity human Vβ2.1 (hVβ2.1) variant called D10, engineered by yeast display, contained 14 mutations beyond that of EP-8, the wild type hVβ2.1 analog that was selected for enhanced stability (R. A. Buonpane et al. (2005) J Mol Biol 353:308-21). Because the yeast display libraries contained stretches of five randomly mutated codons, many of these mutations were likely not involved in affinity increases, but were incorporated in combination with a key mutation(s). In order to determine which of these mutations were significant contributors to the higher affinity interaction with TSST-1, site-directed mutagenesis to create 13 individual single-site mutants from the EP-8 template was performed, including: R10M, F27aT, Q28N, A29I, T30H, E50H, E51Q, S52aF, K53N, T55I, E61V, L72P and I91V (residues 27a and 52a are non-canonical insertions into the hVβ2.1 CDR1 and CDR2 loops, respectively). The R113Q mutation was not made as this position is located on the face of the Vβ domain opposite that of the interface with TSST-1 and is thus unlikely to affect TSST-1 binding.

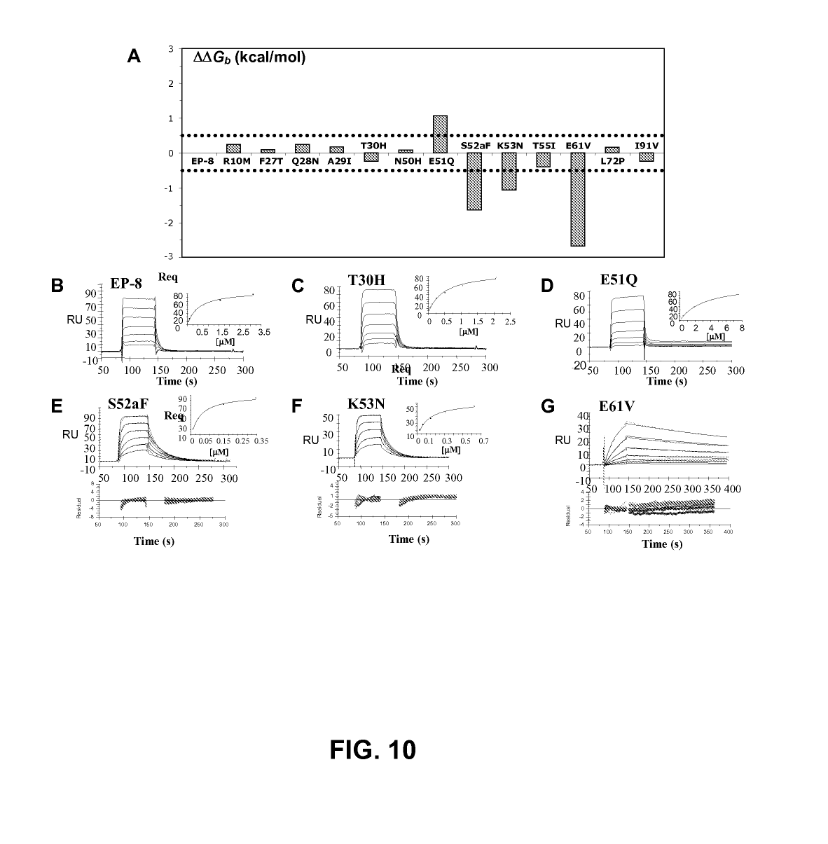

The binding affinities of each of these single-site variants were determined by SPR equilibrium analysis. The differences in binding free energies relative to EP-8 (ΔΔG_{b}) were calculated and a threshold value of 0.5 kcal/mol was used to determine whether a mutation exhibited energetic significance (FIG. 10A). Equilibrium binding analysis of the T30H mutant, as one that does not affect TSST-1 binding significantly, is shown (FIG. 10B-C). Four of the mutants (E51Q, S52aF, K53N and E61V) bound TSST-1 with significantly different affinities than EP-8. Surprisingly, the E51Q mutation bound TSST-1 with significantly lower affinity than did the wild type hVβ2.1 (FIG. 10D). The S52aF (FIG. 10E), K53N (FIG. 10F) and E61V (FIG. 10G) mutations significantly increased the binding affinity for TSST-1.

Although four residues in the CDR1 loop of hVβ2.1 were mutated in the D10 high affinity variant, none of these mutations were energetically significant (FIG. 10A), indicating that the CDR1 loop may not form part of the binding interface with TSST-1. Three of these variant residues, at positions 28, 29 and 30, make contacts with the SAG SpeC in the hVβ2.1-SpeC crystal structure (E. J. Sundberg et al. (2002) Structure 10:687-99). Thus, although TSST-1 and SpeC compete for binding to hVβ2.1 (R. A. Buonpane et al. (2005) J Mol Biol 353:308-21), they likely engage only partially overlapping binding sites on the TCR β chain.

Two Hot Regions in hVβ2.1 for TSST-1 Interaction

Three of the four energetically significant residues (51, 52a and 53) are located in the CDR2 loop, while the remaining important mutation site, at residue 61, is located in framework region 3 (FR3). The remaining mutations, which do not significantly affect TSST-1 binding, are dispersed about the surface of the hVβ2.1 domain. Most, if not all, of these mutations contribute primarily to stabilization and display of the hVβ2.1 protein on the yeast surface (R. A. Buonpane et al. (2005) J Mol Biol 353:308-21; E. V. Shusta et al. (2000) Nat Biotechnol 18:754-9; M. C. Kieke et al. (1999) Proc Natl Acad Sci USA 96:5651-6; M. C. Kieke et al. (2001) J Mol Biol 307:1305-15).

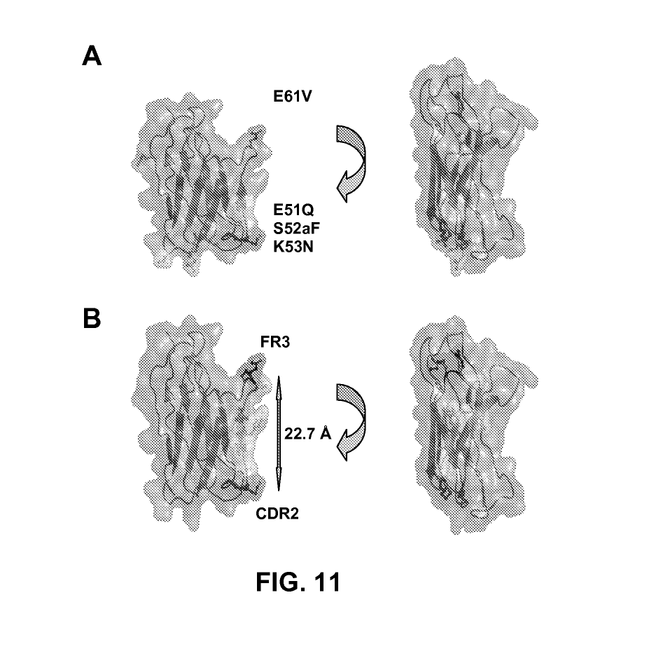

It was previously shown that variant residues at positions 52a, 53 and 61, and the wild type residue at position 62 act as hot spots for interaction with TSST-1 (R. A. Buonpane et al. (2005) J Mol Biol 353:308-21). These residues form two clusters: residues 51, 52a and 53 are located at the apex of the CDR2 loop; residues 61 and 62 are positioned at the end of the turn within FR3 (FIG. 11B). These two clusters are connected by the c″ β-strand of the hVβ2.11 g domain, a secondary structural element common to all TCR variable Ig domains. The distance between the C^{α} atoms of residues 51 and 61 is 22.7 Å (E. J. Sundberg et al. (2002) Structure 10:687-99). According to a structural model of the hVβ2.1-TSST-1 complex (R. A. Buonpane et al. (2005) J Mol Biol 353:308-21), built by taking into account homology to the hVβ2.1-SpeC complex crystal structure (E. J. Sundberg et al. (2002) Structure 10:687-99) and alanine-scanning mutagenesis analysis of both sides of the hVβ2.1-TSST-1 interface (R. A. Buonpane et al. (2005) J Mol Biol 353:308-21; J. K. McCormick et al. (2003) J Immunol 171:1385-92), these clusters are located at the periphery of the interface and are as far apart as possible within that interface. Indeed, the distance between C^{α} atoms of the most peripheral residues in the hVβ2.1-SpeC complex are a comparable 22.1 Å apart (E. J. Sundberg et al. (2002) Structure 10:687-99). These distances also greatly exceed the threshold of 13 Å used to define distinct discontinuous patches within larger protein-protein interfaces (P. Chakrabarti et al. (2002) Proteins 47:334-43). Thus, the energetically significant mutations in the D10 affinity maturation pathway are located in two distinct hot regions.

Saturation Combinatorial Mutational Analysis Reveals a Variant with Higher Affinity Than the Penultimate Yeast Display Variant

Once the hVβ2.1 mutations that contributed significantly to TSST-1 binding were identified, saturation combinatorial mutational analysis was carried out in order to dissect the additive and cooperative energetic properties between these residues. In this approach, hVβ2.1 mutants that incorporated every possible combination of wild type and mutant residues at each of the four variant positions, residues 51, 52a, 53 and 61 were made. This amounts to a total of sixteen (2⁴) distinct hVβ2.1 mutant proteins, including EP-8 (the wild type analog), the four single-site mutants described above, six double mutants, four triple mutants and one variant that simultaneously incorporated all four mutations.

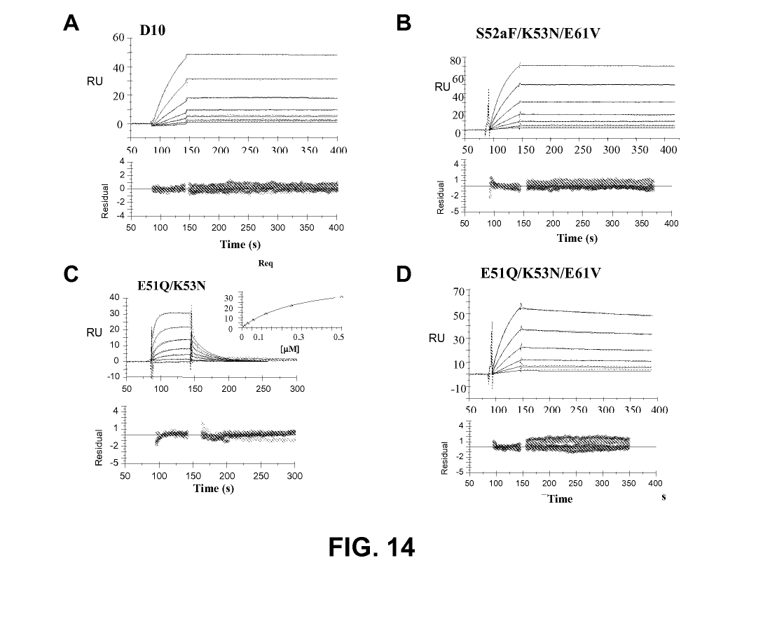

It was found that in another TCR Vβ domain-SAG model system that the affinity maturation pathway was restricted by negative cooperativity between two residues, each of which significantly increased the Vβ domain binding affinity for the SAG binding partner individually (J. Yang et al. (2003) J Biol Chem 278:50412-21). Structural analysis of this affinity maturation pathway showed that this negative cooperativity was a result of two CDR2 mutations that caused conformational changes in the CDR2 loop (S. Cho et al. (2005) Structure (Camb) 13:1775-1787). Because the unfavorable E51Q mutation was retained in all of the clones from the final (and highest affinity) sort of the yeast display affinity maturation, which includes the highest affinity variant D10 (R. A. Buonpane et al. (2005) J Mol Biol 353:308-21; S. Cho et al. (2005) Structure (Camb) 13:1775-1787), it would seem likely that this mutation in combination with other mutations in D10 would act in a positively cooperative manner to increase the overall affinity for TSST-1. It was found, on the contrary, that the S52aF/K53N/E61V triple mutant, which incorporates only the three mutations that significantly increase TSST-1 affinity individually and not the E51Q mutation, binds with nearly a log-fold higher affinity than does D10 (Table I and FIG. 14A-B). Not only is the detrimental energetic effect of the E51Q mutation absent in this triple mutant, but this combination of mutations is also positively cooperative (see below).

Because the dissociation kinetics of these interactions approach the measurement limits of current SPR technology (P. Schuck et al. (1999) Curr Opin Prot Sci 17:20.2.1-20.2.22), analogous binding experiments for the D10-TSST-1 and S52aF/K53N/E61V-TSST-1 interactions were performed in which the dissociation time was extended from 5 minutes to 2 hours, a 24-fold longer dissociation time (data not shown). Both the long dissociation times and the dissociation rate constant of the S52aF/K53N/E61V-TSST-1 interaction are similar to those of several high affinity antibody-antigen interactions analyzed by SPR (A. W. Drake et al. (2004) Anal Biochem 328:35-43). No significant differences in the measured off-rates were observed for these interactions, relative to the shorter dissociation time experiments shown in FIG. 14A-B. The S52aF/K53N/E61V triple mutant has a very high affinity to TSST-1 (K_{D} value of 27 pM), and can be used as a therapeutic molecule for TSST-1-mediated disease.

Mutations within the CDR2 Hot Region are Cooperative

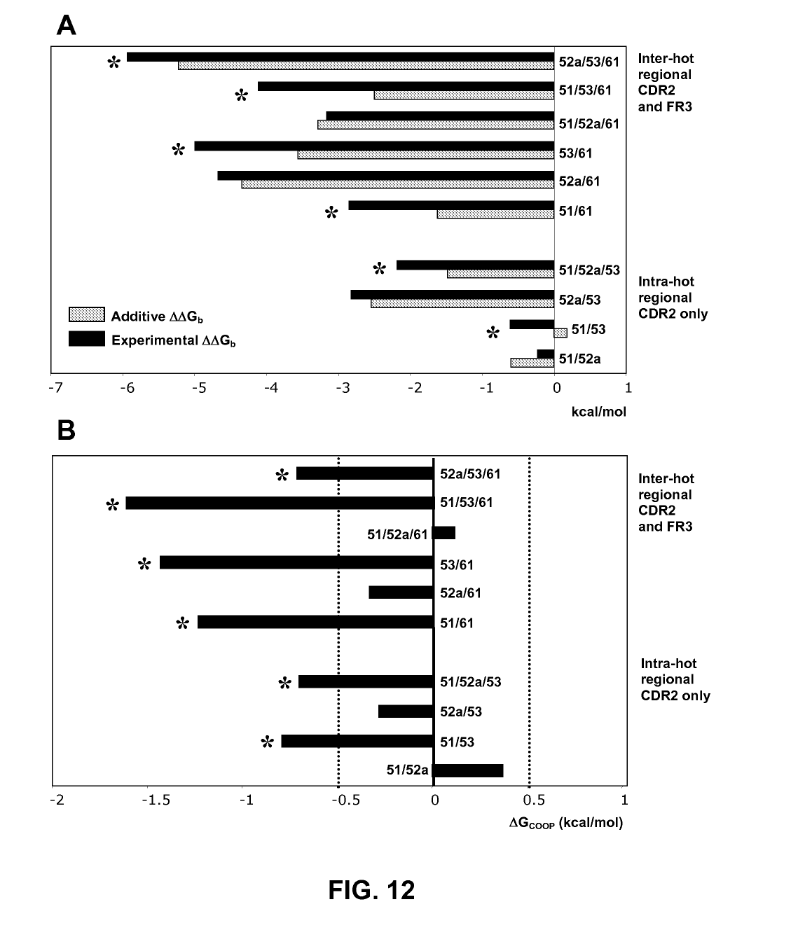

To determine whether mutations within a hot region of the hVβ2.1 domain are energetically additive or cooperative, the TSST-1 binding affinities of each of the combinatorial variants incorporating all energetically significant mutations in the CDR2 loop were determined. This included the double mutants E51Q/S52aF, E51Q/K53N, S52aF/K53N and the triple mutant E51Q/S52aF/K53N. Kinetic parameters (in all cases except the E51Q/S52aF variant, for which the dissociation kinetics were too fast to measure accurately by SPR), affinities, ΔΔG_{b} values relative to EP-8, and ΔG_{COOP} values (calculated as the difference between the summation of the changes in binding free energies of the single-site mutants and the experimental changes in binding free energies of the corresponding combinatorial mutant) are listed in Table 2. A representative SPR sensorgram for the E51Q/K53N double mutant binding to TSST-1 is shown in FIG. 14C. The experimental ΔΔG_{b} values for these combinatorial variants and the ΔΔG_{b} values of the summation of the corresponding single-site mutations are shown in FIG. 12A.

Using the previous threshold of |ΔG_{COOP}|≧0.5 kcal/mol for energetic cooperativity (J. Yang et al. (2003) J Biol Chem 278:50412-21), it was found that mutations at residues 51 and 53 exhibit a moderate degree of positive cooperativity within the CDR2 hot region (FIG. 12B). This result differs from previous dissection of additive and cooperative energetics in the mVβ8.2-SEC3 affinity maturation system (J. Yang et al. (2003) J Biol Chem 278:50412-21), in which residues within the CDR2 loop were negatively cooperative. These findings do, however, reflect results from other molecular systems in which intra-hot regional mutations frequently exhibit positive cooperativity (O. Keskin et al. (2005) J Mol Biol 345:1281-94; D. Reichmann et al. (2005) Proc Natl Acad Sci USA 102:57-62). The combination of mutations within the FR3 hot region, E61V and K62A, were not analyzed for cooperativity as only residue 61 was altered in the affinity maturation pathway, while the other mutation originated as an alanine scanning mutant (R. A. Buonpane et al. (2005) J Mol Biol 353:308-21).

### Mutations Between the CDR2 and FR3 hot Regions are Positively Cooperative

The same strategy as outlined above to determine whether mutants that incorporate combinations of mutations from both the CDR2 and FR3 hot regions were additive or cooperative was employed. This entailed measuring TSST-1 binding parameters from the double mutants E51Q/E61V, S52aF/E61V, K53N/E61V, the triple mutants E51Q/S52aF/E61V, E51Q/K53N/E61V and S52aF/K53N/E61V, and the quadruple mutant E51Q/S52aF/K53N/E61V. Kinetic parameters, affinities, ΔΔG_{b} and ΔG_{COOP} values for these mutants are listed in Table 2. A representative SPR sensorgram for the E51Q/K53N/E61V triple mutant binding to TSST-1 is shown in FIG. 14D. The experimental ΔΔG_{b} values for these combinatorial variants and the ΔΔG_{b} values of the summation of the corresponding single-site mutations are shown in FIG. 12A.

These data show that mutation at residue 61 in the FR3 hot region is highly positively cooperative (|G_{COOP}|≧1.0 kcal/mol) with either or both residues 51 and 53 in the CDR2 hot region (FIG. 12B). Compared to the measured cooperativity within the CDR2 hot region, the cooperative energetics between residues from both hot regions are significantly greater in magnitude. For instance, the largest cooperative effect observed is that of the E51Q/K53N/E61V triple mutant, the combinatorial mutant incorporating mutations at FR3 residue 61 and the two CDR2 residues with which it is cooperative, 51 and 53. The difference in experimental versus additive changes in binding free energies for simultaneous mutation at these positions is −1.61 kcal/mol. In comparison, the E51Q/K53N variant, the most cooperative intra-hot regional combinatorial mutant, exhibits a ΔG_{b} value of only −0.79 kcal/mol (FIG. 12B).

The combination of mutations at residue 61 (FR3 hot region) with both residues 52 and 53 (CDR2 hot region) is moderately cooperative (|ΔG_{COOP}|≧0.5 kcal/mol). The S52aF/K53N/E61V triple mutant binds TSST-1 with a K_{D} of 27 pM, while D10, the penultimate yeast display variant, binds TSST-1 with a K_{D} of 180 pM. In the absence of the exhibited positive cooperativity, it was calculated that the triple mutant would instead bind TSST-1 with a K_{D} of 89 pM, intermediate to the TSST-1 affinities of the S52aF/K53N/E61V mutant and D10, and more than three-fold tighter than if these mutations were strictly additive.

Previous reports (O. Keskin et al. (2005) J Mol Biol 345:1281-94; D. Reichmann et al. (2005) Proc Natl Acad Sci USA 102:57-62) had suggested that, contrary to mutations within a hot region, those between distinct hot regions are not cooperative, but strictly additive. These results suggest that this is not the case for all protein-protein interactions.

The Protein Core as an Energetic Sink that Regulates Cooperativity Between Hot Regions

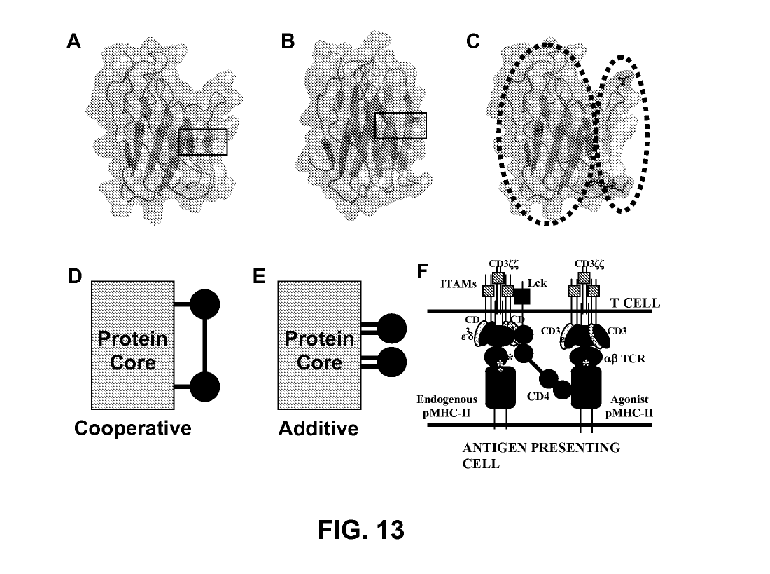

The two hot regions in the hVβ2.1 domain for TSST-1 binding are located before and after the c″ β-strand of the Ig domain. This strand has been shown to participate in a strand-switching event in TCR Vβ domains. In most Vβ domains, such as in hVβ2.1, the c″ β-strand is hydrogen bonded to the preceding c′ β-strand (FIG. 13A). In some Vβ domains, however, the c″ β-strand is hydrogen bonded to the following d β-strand. An example of this strand-switching is shown in FIG. 13B for the murine Vβ2 domain (D. Housset et al. (1997) Embo J 16:4205-16).

The implications of strand-switching for the structure of the TCR Vβ domain and the interaction of proteins with this region of the Vβ domain are two-fold. First, the c″ β-strand can be considered to lie outside of the hydrogen bonded β-strand network that forms the hVβ2.1 protein core. This can be seen by comparison of FIGS. 13A and 13B, and is most clearly depicted in FIG. 13C, in which the mutated residues in both the CDR2 and FR3 hot regions, as well as the connecting c″ β-strand, are highlighted. Second, the c″ β-strand, relative to other β-strands in the TCR Vβ Ig domain, has a propensity for flexibility.

The CDR2 and FR3 hot regions in hVβ2.1 may thus be considered as two balls connected by a string outside of the protein core (FIG. 13D). In such a situation, it is possible that cooperative energetics are a result of conformational changes transmitted along the c″ β-strand (i.e., the string) from one hot region to the other. Although the distance between the two hot regions spans the breadth of the molecular interface, and is thus as large as possible for the given protein-protein interaction, the connecting residues are positioned outside of the protein core, and thus, not integrally involved in forming the intramolecular contacts that stabilize the protein. Such a positioning of the hot region intervening sequence along the exterior of the protein core may increase the propensity for global conformational changes to be transmitted from one hot region to another, even though the c″ β-strand is hydrogen bonded to the c′ β-strand, itself part of the protein core, allowing for cooperative energetics. Cooperativity may arise in the hVβ2.1-TSST-1 system by a number of mechanisms, some of which have been observed in other molecular systems, including: (1) a tightening of the hVβ2.1 molecular surface upon TSST-1 binding, reminiscent of G protein-coupled receptors (D. H. Williams et al. (2004) J Mol Biol 340:373-83; D. H. Williams et al. (2004) Angew Chem Int Ed Engl 43:6596-616); or (2) the entropic costs of TSST-1 binding and conformational changes being not strictly additive, such as in the homodimerization of glycopeptide antibiotics (S. Jusuf et al. (2002) J Am Chem Soc 124:3490-1). These possibilities need to be tested by structural and thermodynamic studies, analogous to those that have been carried out to define the molecular basis for negative cooperativity within a hot region for another TCR Vβ-SAG interaction (J. Yang et al. (2003) J Biol Chem 278:50412-21; S. Cho et al. (2005) Structure (Camb) 13:1775-1787).

In contrast, it is suggested that hot regions for which the connecting residues are integrally involved in the formation of the protein core (FIG. 13E) result in additive energetics, even when the distance on the molecular surface between hot regions is short. These types of hot regions are most common in protein interfaces, and are representative of the hot regions in the TEM1-BLIP complex, the only other protein complex for which rigorous mutational analysis has been applied to address the question of additive versus cooperative energetics between hot regions, and for which it was found that inter-hot regional mutations were merely additive (D. Reichmann et al. (2005) Proc Natl Acad Sci USA 102:57-62). Thus, the protein core is believed to serve to regulate cooperative energetics between hot regions by absorbing the energy from any conformational changes being transmitted from one hot region to another.

### Long-Range Cooperative Binding Effects Suggest Plausible Mechanisms for Initiation of T Cell Signaling

The finding of long-range cooperative effects in a V domain of the TCR may provide a framework for understanding how a multimeric TCR/CD3 complex could be influenced by ligand binding. In order to initiate T cell activation, the αβ TCR binds peptide antigens presented on the cell surface by major histocompatibility molecules (MHC). These molecules on the T cell and antigen presenting cell (APC) are sequestered into an immunological synapse, into which other costimulatory molecules are directed during the signaling events (D. J. Irvine et al. (2002) Nature 419:845-9: C. Wulfing et al. (2002) Nat Immunol 3:42-7). For CD4⁺ T cells, these include the coreceptor CD4 and numerous endogenous, or self, peptide-MHC (pMHC) complexes. Although T cells are able to detect a single pMHC on the APC surface (D. J. Irvine et al. (2002) Nature 419:845-9; M. A. Purbhoo et al. (2004) Nat Immunol 5:524-30), monomeric ligands have been shown to be incapable of stimulating CD4⁺ T cells (J. R. Cochran et al. (2000) Immunity 12:241-50; J. J. Boniface et al. (1998) Immunity 9:459-66). Self pMHC, by themselves unable to stimulate T cell activation, contribute significantly to T cell recognition of both CD4⁺ (C. Wulfing et al. (2002) Nat Immunol 3:42-7) and CD8⁺ cells (P. P. Yachi et al. (2005) Nat Immunol 6:785-92). CD4, when bound to an agonist pMHC complex, is incapable of engaging this agonist pMHC-specific TCR (J. H. Wang et al. (2001) Proc Natl Acad Sci USA 98:10799-804), but instead appears to orient the tyrosine kinase Lck to enable self pMHC to trigger many TCR (Q. J. Li et al. (2004) Nat Immunol 5:791-9). This and other evidence has led to the proposal of the “pseudodimer” model for the initiation of T cell activation (FIG. 13F), in which heterodimers of agonist and endogenous pMHC, stabilized by CD4, initiate T cell activation and control the sensitivity of the T cell response (D. J. Irvine et al. (2002) Nature 419:845-9; Q. J. Li et al. (2004) Nat Immunol 5:791-9; M. Krogsgaard et al. (2005) Nature 434:238-43).

Other factors, however, are also important for TCR signaling. The TCR associates with CD3εδ and εδ heterodimers and the ζζ homodimer (FIG. 13F). NMR and crystal structures of CD3δε and εγ (Z. J. Sun et al. (2001) Cell 105:913-23; Z. Y. Sun et al. (2004) Proc Natl Acad Sci USA 101:16867-72; K. L. Arnett et al. (2004) Proc Natl Acad Sci USA 101:16268-73) have suggested that the TCR/CD3 complexes may act as a rigid transduction module and that a piston-like displacement upon interaction with pMHC could be the basis for the intracellular phosphorylation events that initiate activation. Conformational changes in the TCR upon pMHC interaction, as determined by a correlation between heat capacity changes and T cell stimulatory levels, may also play a role in T cell activation (M. Krogsgaard et al. (2003) Mol Cell 12:1367-78).

It remains unclear how all of these effects control T cell signaling. Long-range positive cooperative binding effects within the TCR variable domain, such as those observed in the system of TCR Vβ variants interacting with TSST-1, supports the possibility that ligand binding by CDRs could influence, through a conformational change, the association of other proteins with framework regions of the V domain. If such effects occur in the TCR upon engagement with pMHC and/or coreceptors, the energetic transmission between the CDR loops (white asterisk in FIG. 13F) and the “top” of the FR (black asterisk in FIG. 13F) provide a number of plausible mechanisms for augmenting T cell activation and sensitivity. Binary interactions between endogenous pMHC, coreceptors, TCR, and CD3 molecules are relatively weak, and a mechanism such as cooperativity could have profound impacts on the association of these molecules into higher-order associations required for signaling.

For instance, CD4-TCR interactions could be enhanced by TCR interaction with endogenous pMHC (FIG. 13F), resulting in a conformational change that leads to coordinate Lck and phosphorylating CD3 immunoreceptor tyrosine activation motifs (ITAMs). Although CD4 does not appear to affect the binding of TCR-pMHC when bound to the same pMHC (Y. Xiong et al. (2001) J Biol Chem 276:5659-67), CD4-TCR interactions could stabilize the otherwise weak interactions of CD4 with an endogenous pMHC complex. This could result in stabilization of the entire TCR/endogenous pMHC/CD4/TCR/agonist pMHC signaling complex. Alternatively, pMHC binding by the TCR could influence the TCR-CD3 interaction, resulting in either redistributions of the CD3 complex, or altered association with individual CD3 subunits. The latter might even occur indirectly through a TCR constant region interaction. Given the relatively weak associations among these TCR/CD3/CD4 (or CD8) assemblies, modest conformational effects could yield significant changes in their associations.

### Implications for Protein-Protein Interaction Prediction and Inhibition

A fundamental lack of understanding of cooperative energetics is one of the major impediments to formulating with greater accuracy algorithms for protein-protein interaction prediction. If cooperativity existed only within hot regions, and not between them, the task of accurately predicting the binding parameters for protein complexes would be greatly simplified. These results suggest that this may be an overly-generalized representation of macromolecular interfaces and that a broader consideration of cooperativity within protein-protein interactions, while more technically and computationally demanding, may ultimately lead to more accurate predictive algorithms. It also appears from these results that only a subset of hot regions, such as those that are connected to one another by structural elements that do not form part of the protein core, may need to be considered as potentially cooperative. Additional mutational analyses of diverse protein-protein interaction systems, such as those presented for the TEM1-BLIP complex (D. Reichmann et al. (2005) Proc Natl Acad Sci USA 102:57-62) and that are shown here, will be required to confirm this.

Because protein-protein interactions are pervasive in biological processes, they are also important therapeutic targets. The development of small molecule inhibitors of such interactions has proven difficult (M. R. Arkin et al. (2004) Nat Rev Drug Discov 3:301-17), largely due to the relatively planar nature of these interfaces, which tend not to present well-defined binding pockets. The presence of hot spots and hot regions within protein interfaces provide possible sites at which potent small molecule inhibitors may bind to effectively block the association of much larger molecules. Indeed, small peptides selected by phage display generally bind their protein binding partners at hot spots (S. S. Sidhu et al. (2003) Chembiochem 4:14-25), and the discovery of small molecules that inhibit the interaction of B7-1 with CD28 and modulate T cell activation, and in which the drug binds at a hot spot, has been reported (N. J. Green et al. (2003) Bioorg Med Chem 11:2991-3013; D. V. Erbe et al. (2002) J Biol Chem 277:7363-8). If certain distinct hot regions may be linked energetically, as these results suggest, the potency of a small molecule inhibitor that targets a cooperative hot region may be amplified relative to a small molecule that targets a hot region that is strictly additive. This has important ramifications for the choice of which hot region within a protein-protein interaction to target for small molecule inhibition, for instance, by structure-based drug design.

### Materials and Methods

### Protein Production

All hVβ2.1 variants were expressed in E. coli and refolded in vitro from inclusion bodies as described previously for mVβ8.2 domain variants (J. Yang et al. (2003) J Biol Chem 278:50412-21). The wild type TSST-1 gene (tst) was PCR amplified from pCE107 (J. K. McCormick et al. (2003) J Immunol 171:1385-92), and cloned into the NcoI and BamHI sites of pET41a (Novagen, Madison, Wis.). The forward primer amplified tst lacking the region encoding the signal peptide, and additionally, engineered a tobacco etch virus (TEV) protease cleavage site (ENLYFQG) upstream of the tst gene, which when cloned, replaced the pET41a enterokinase cleavage site (DDDDK). The TSST-1 protein was expressed in E. coli BL21(DE3) (Novagen, Madison, Wis.), purified by Ni²⁺-column chromatography using the His₆ tag, cleaved with autoinactivation-resistant His₇:TEV as described (R. B. Kapust et al. (2001) Protein Eng 14:993-1000), and further purified by size exclusion chromatography.

### Mutagenesis

All mutagenesis was performed using the QuikChange site-directed mutagenesis kit according to the manufacturer's instructions, using the pT7-7/EP-8 expression vector as a template (R. A. Buonpane et al. (2005) J Mol Biol 353:308-21). For saturation combinatorial mutagenesis, the order of mutations was important, in that the complementary sites for oligonucleotide primers targeted to the three codons of interest in the CDR2 loop were overlapping, and thus, changed according to the sequence of the mutagenesis events. The double, triple and quadruple mutants were generated by sequential rounds of site-directed mutagenesis, such that the lower order mutation vectors were always used as templates for the higher order mutants.

### Surface Plasmon Resonance Binding Analysis

The interaction of hVβ2.1 variants with immobilized TSST-1 was measured by SPR equilibrium and, where applicable, kinetic analyses using a Biacore 3000 SPR instrument (Biacore), as described previously (R. A. Buonpane et al. (2005) J Mol Biol 353:308-21). Briefly, 100-500 resonance units (RU) of TSST-1 were immobilized to a CM-5 sensorchip (Biacore) by amine coupling. Staphylococcal enterotoxin B (SEB) in an equivalent surface density was used as the control surface. The hVβ2.1 variants were injected at a flow rate of 25 μl/min, serially diluted in 10 mM Hepes buffer containing 150 mM sodium chloride, 3.4 mM EDTA and 0.005% surfactant P-20, interspersed with pulsed injections of 10 mM HCl to regenerate both surfaces. SPR data for association (kₐ) and dissociation (k_{d}) rates, as well as the dissociation constant (K_{D}) were determined by globally fitting all data from multiple injected hVβ2.1 variant concentrations to a simple 1:1 Langmuir binding model using the BIAevaluation 4.1 software.

TABLE 2
Equilibrium binding analysis hVβ2.1
single-site mutants interacting with TSST-1

| | K_{A} | K_{D} | ΔG_{b} | ΔΔG_{b} |
|---|---|---|---|---|
| | M⁻¹ | M | kcal/mol | kcal/mol |
| EP-8 | 1.7 × 10⁶ | 6 × 10⁻⁷ | −8.48 | 0.00 |
| R10M | 1.1 × 10⁶ | 9 × 10⁻⁷ | −8.24 | 0.24 |
| F27aT | 1.4 × 10⁶ | 7 × 10⁻⁷ | −8.39 | 0.09 |
| Q28N | 1.1 × 10⁶ | 9 × 10⁻⁷ | −8.24 | 0.24 |
| A29I | 1.3 × 10⁶ | 8 × 10⁻⁷ | −8.31 | 0.17 |
| T30H | 2.5 × 10⁶ | 4 × 10⁻⁷ | −8.72 | −0.24 |
| N50H | 1.7 × 10⁶ | 6 × 10⁻⁷ | −8.56 | 0.08 |
| E51Q | 0.3 × 10⁶ | 36 × 10⁻⁷ | −7.42 | 1.06 |
| S52aF | 27.8× | 0.38 × 10⁻⁷ | −10.12 | −1.64 |
| K53N | 26.3× | 1.1 × 10⁻⁷ | −9.55 | −1.07 |
| T55I | 3.3 × 10⁶ | 3 × 10⁻⁷ | −8.89 | −0.41 |
| E61V | 154 × 10⁶ | 0.065× | −11.17 | −2.68 |
| L72P | 1.3 × 10⁶ | 8 × 10⁻⁷ | −8.32 | 0.16 |
| I91V | 2.5 × 10⁶ | 4 × 10⁻⁷ | −8.72 | −0.24 |

Equilibrium affinity constants (K_{A}) were derived by steady-state affinity analysis of surface plasmon resonance data. Dissociation constants (K_{D}) and free energies of binding (ΔG_{b}) were calculated from K_{A}, using the equation ΔG_{b} = − RTInK_{A}. The changes in free energy gained or lost (ΔΔGb) were determined using the free energy of EP-8, the wild type hVβ2.1 analog, as a reference. Single-site mutants that confer significant energetic changes relative to EP-8 are highlighted.

TABLE 3
Kinetic binding analysis for hVβ2.1 single-site and combinatorial variants interacting with TSST-1

| | | | | | | |
|---|---|---|---|---|---|---|
| kₐ | k_{d} | K_{A} | K_{D} | ΔG_{b} | ΔΔG_{b} | ΔG_{COOP} |
| M⁻¹s⁻¹ (10⁵) | s⁻¹ (10⁻³) | M⁻¹ | M | kcal/mol | kcal/mol | kcal/mol |

Single-site mutants

| | | | | | | | |
|---|---|---|---|---|---|---|---|
| EP-8 (Wild Type) | ND | ND | 1.7 × 10⁶ | 6.0 × 10⁻⁷ | −8.48 | 0 | NA |
| E51Q | ND | ND | 2.8 × 10⁵ | 3.6 × 10⁻⁶ | −7.42 | 1.06 | NA |
| S52aF | 10.31 ± 0.15 | 38 ± 0.23 | 2.72 ± 0.40 × 10⁷ | 3.68 ± 0.45 × 10⁻⁸ | −10.14 | −1.66 | NA |
| K53N | 3.99 ± 0.07 | 54 ± 0.43 | 7.39 ± 0.70 × 10⁶ | 1.35 ± 0.12 × 10⁻⁷ | −9.36 | −0.88 | NA |
| E61V | 7.07 ± 0.09 | 4.6 ± 0.07 | 1.54 ± 0.31 × 10⁸ | 6.51 ± 0.60 × 10⁻⁹ | −11.16 | −2.68 | NA |

CDR2 intra-hot regional mutants

| | | | | | | | |
|---|---|---|---|---|---|---|---|
| E51Q/S52aF | ND | ND | 2.5 × 10⁶ | 4.1 × 10⁻⁷ | −8.71 | −0.23 | 0.37 |
| E51Q/K53N | 3.13 ± 0.18 | 67.7 ± 0.13 | 4.62 ± 0.42 × 10⁶ | 2.16 ± 0.38 × 10⁻⁷ | −9.09 | −0.61 | −0.79 |
| S52aF/K53N | 7.91 ± 0.03 | 4.05 ± 0.003 | 1.95 ± 0.40 × 10⁸ | 5.12 ± 0.73 × 10⁻⁹ | −11.30 | −2.82 | −0.28 |
| E51Q/S52aF/K53N | 13.4 ± 0.08 | 20.41 ± 0.01 | 6.57 ± 0.28 × 10⁷ | 1.52 ± 0.08 × 10⁻⁸ | −10.66 | −2.18 | −0.70 |

CDR2/FR3 inter-hot regional mutants

| | | | | | | | |
|---|---|---|---|---|---|---|---|
| E51Q/E61V | 3.61 ± 0.02 | 1.76 ± 0.002 | 2.05 ± 0.07 × 10⁸ | 4.87 ± 0.20 × 10⁻⁹ | −11.33 | −2.85 | −1.23 |
| S52aF/E61V | 7.01 ± 0.05 | 0.16 ± 0.002 | 4.41 ± 0.42 × 10⁹ | 2.28 ± 0.67 × 10⁻¹⁰ | −13.15 | −4.67 | −0.33 |
| K53N/E61V | 12.3 ± 0.03 | 0.16 ± 0.001 | 7.58 ± 0.80 × 10⁹ | 1.32 ± 0.16 × 10⁻¹⁰ | −13.47 | −4.99 | −1.43 |
| E51Q/S52aF/E61V | 3.38 ± 0.02 | 0.98 ± 0.001 | 3.45 ± 0.61 × 10⁸ | 2.91 ± 0.52 × 10⁻⁹ | −11.64 | −3.16 | 0.12 |
| E51Q/K53N/E61V | 8.2 ± 0.03 | 0.49 ± 0.001 | 1.71 ± 0.17 × 10⁹ | 5.83 ± 0.51 × 10⁻¹⁰ | −12.59 | −4.11 | −1.61 |
| S52aF/K53N/E61V | 8.82 ± 0.02 | 0.024 ± 0.001 | 3.69 ± 0.60 × 10¹⁰ | 2.71 ± 0.59 × 10⁻¹¹ | −14.41 | −5.93 | −0.71 |
| E51Q/S52aF/K53N/E61V | 9.37 ± 0.03 | 0.19 ± 0.001 | 6.57 ± 0.96 × 10⁹ | 1.51 ± 0.29 × 10⁻¹⁰ | −13.39 | −4.91 | −0.57 |

Kinetic parameters of binding (kₐ and k_{d}) were determined by global fitting to a 1:1 binding model of all data from both association and dissociation phases of multiple concentrations of the hVβ2.1 single-site and combinatorial variants over a surface plasmon resonance sensorchip surface onto which TSST-1 had been immobilized. Affinity (K_{A}) and dissociation constants (K_{D}) were determined from the ratios of the association and dissociation rates. Free energies of binding (ΔG_{b}) were calculated from K_{A}, using the equation ΔG_{b} = −RTlnK_{A}. The changes in free energy gained or lost (ΔΔG_{b}) were determined using the free energy of EP-8, the wild type hVβ2.1 analog, as a reference. The cooperative free energy (ΔG_{COOP}) was calculated as the difference between the summation of the changes in the free energies of binding of the single-site mutants and the change in the free energy of binding of the corresponding combinatorial mutant.

### Example 3

### Soluble Vβ Having High-Affinity for Staphyloccal Enterotoxin B (SEB)

### In Vitro Neutralization of SEB-Mediated Activity by Soluble High-Affinity Vβ Regions

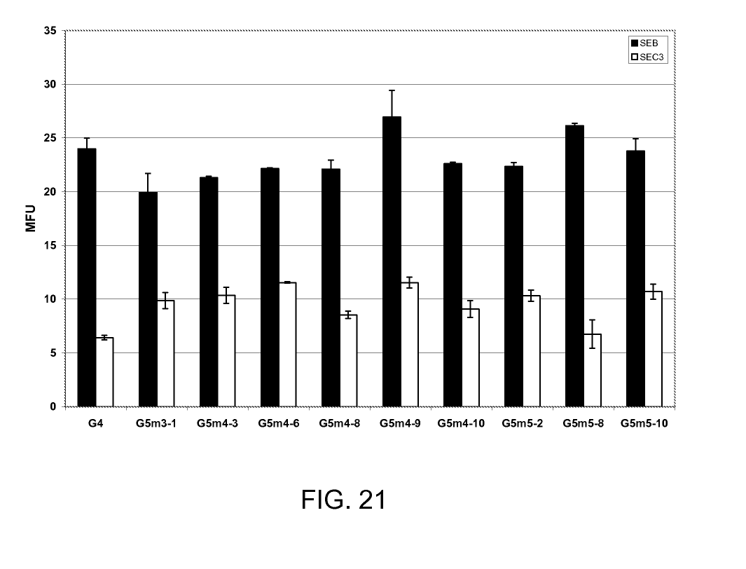

FIG. 21 shows cross-reactivity of mVβ8.2 clones generated for high-affinity to SEB. Yeast clones expressing the indicated Vβ domain on their surface were incubated for one hour on ice with 200 nM biotinylated SEB or SEC3. Binding was measured by flow cytometry. Table 4 shows representative kinetic and affinity parameters.

TABLE 4
Binding parameters for affinity matured mVβ8.2 variants interacting
with SEB as measured by surface plasmon resonance analysis¹

| | kₐ (M⁻¹s⁻¹) | k_{d} (s⁻¹) | K_{A} (M⁻¹) | K_{D} (M) |
|---|---|---|---|---|
| G2 | 3.81 ± 0.26 × 10⁶ | 2.48 ± 0.23 × 10⁻³ | 1.54 ± 0.04 × 10⁹ | 6.49 ± 0.16 × 10⁻¹⁰ |
| G4 | 3.66 ± 0.29 × 10⁶ | 7.13 ± 0.44 × 10⁻⁴ | 5.13 ± 0.09 × 10⁹ | 1.95 ± 0.04 × 10⁻¹⁰ |
| G5m4-3 | 2.99 ± 0.27 × 10⁶ | 2.47 ± 0.40 × 10⁻⁴ | 1.23 ± 0.18 × 10¹⁰ | 8.20 ± 1.24 × 10⁻¹¹ |
| G5m4-6 | 3.16 ± 0.40 × 10⁶ | 1.91 ± 0.14 × 10⁻⁴ | 1.65 ± 0.11 × 10¹⁰ | 6.09 ± 0.42 × 10⁻¹¹ |
| G5m4-8 | 3.44 ± 0.20 × 10⁶ | 1.64 ± 0.08 × 10⁻⁴ | 2.11 ± 0.05 × 10¹⁰ | 4.75 ± 0.12 × 10⁻¹¹ |
| G5m4-9 | 2.50 ± 0.16 × 10⁶ | 2.32 ± 0.19 × 10⁻⁴ | 1.08 ± 0.06 × 10¹⁰ | 9.31 ± 0.50 × 10⁻¹¹ |
| G5m4-10 | 3.04 ± 0.41 × 10⁶ | 2.09 ± 0.43 × 10⁻⁴ | 1.49 ± 0.18 × 10¹⁰ | 6.84 ± 0.87 × 10⁻¹¹ |

¹Binding parameters derived from three independent binding analysis for each biomolecular interaction by global curve-fitting kinetic analysis using the BIAevaluation 4.1 software

### Example 4

### Affinity Maturation of Vβ8 by Yeast Display

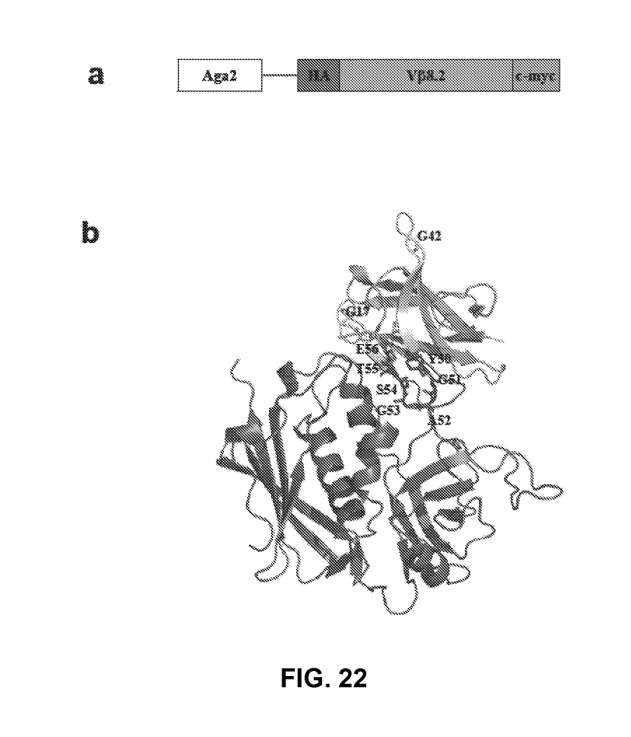

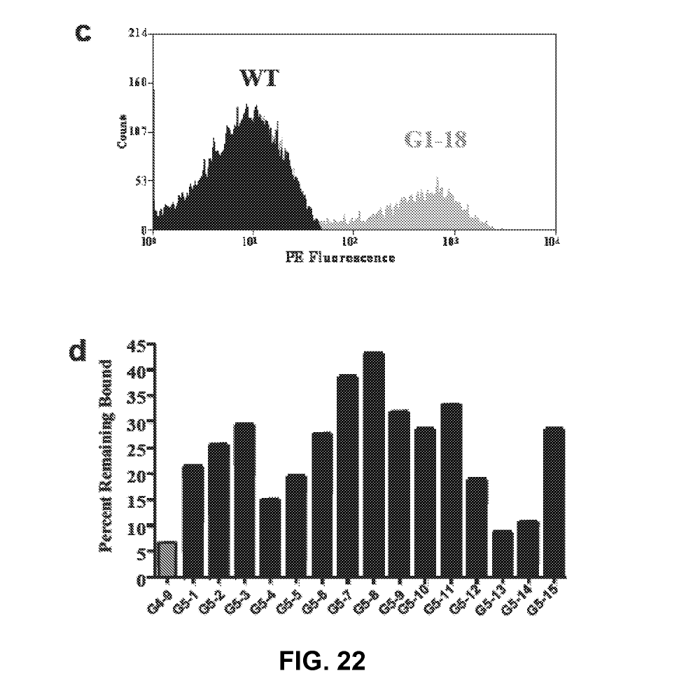

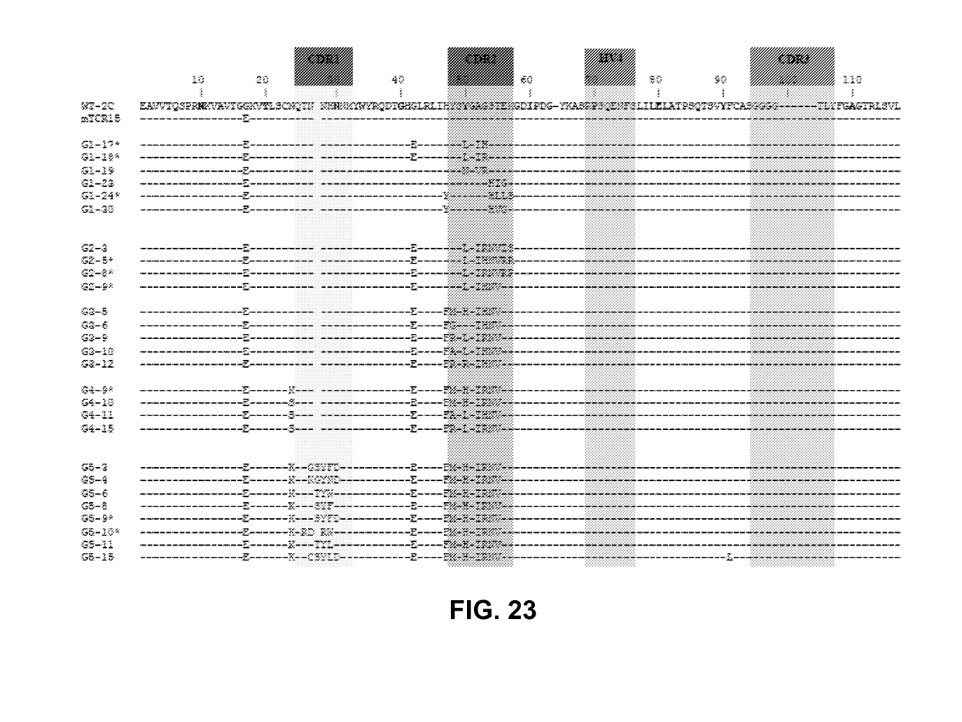

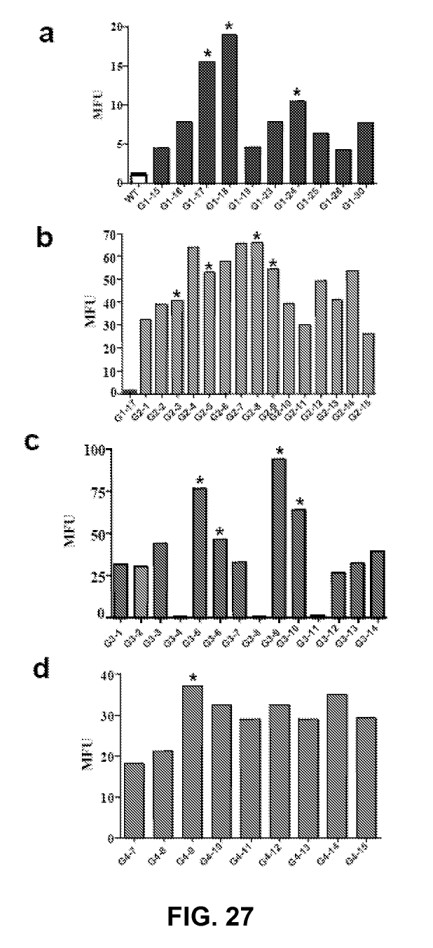

To engineer high-affinity receptor antagonists for SEB, the mouse Vβ8.2 domain was cloned into the yeast display vector, pCT202 (FIG. 22A). A detailed description of the engineering by yeast display is provided below. Affinity maturation of the Vβ8 involved five successive generations (G1 through G5) of libraries containing various site-directed or random mutations, each followed by selection with biotinylated SEB and high-speed flow sorting. The mutagenic libraries included regions at the SEB:Vβ8 interface (FIG. 22B) as follows: G1: one half of CDR2, G2: the other half of CDR2, G3: framework region 2, G4: random mutagenesis, G5: CDR1. Generations 1 through 4 libraries were selected with successively lower concentrations of SEB. Various clones from each library were screened for binding to SEB by flow cytometry and all were positive for binding to SEB, whereas wild-type Vβ is undetectable at this concentration (FIG. 22C and FIG. 27). Sequences of selected mutants from each generation are shown in FIG. 23. Conserved sequence motifs of affinity-matured clones, and their possible mechanisms of action, are described below.

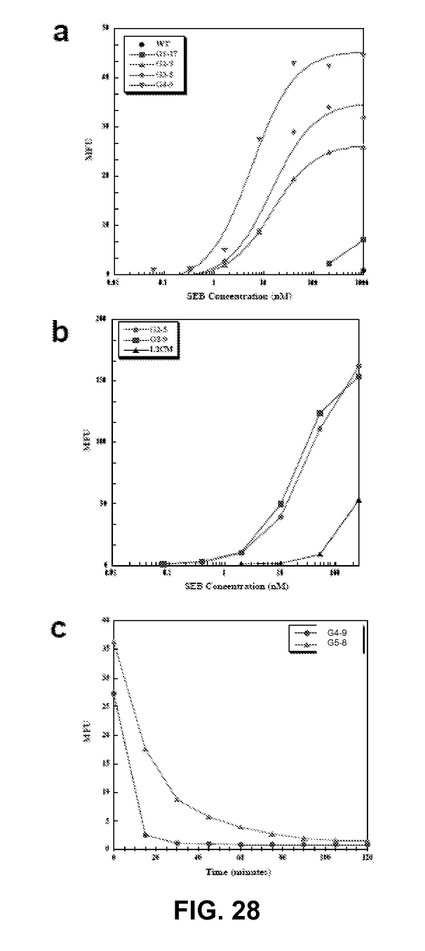

In order to further enhance affinity of the interaction, the fifth generation involved ‘extension’ libraries in CDR1, and an off-rate based selection scheme. The CDR1 ‘extension’ engineering was based on the premise that SEB is only 7A from CDR1 of Vβ8 and that SpeC contacts the CDR1 of human Vβ2.1, which contains an extra amino acid compared to Vβ8. Three yeast display libraries were made with different CDR1 lengths: ΔCDR1 (residues 26-30 randomized), CDR1+1 (residues 27-30 randomized, one amino acid inserted at position 27a), and CDR1+2 (residues 27a-30, with two amino acids inserted at positions 27a and b). Mutant G4-9 was used as template and the three libraries were pooled at equal ratios prior to selection, using off-rate based sorting. Fifteen clones from the final selection were screened for the amount of bound ligand remaining after four hours at 25° C. (FIG. 22D). All clones showed improvements over clone G4-9, which had <10% bound ligand remaining. In contrast, clone G5-8, had almost 50% bound SEB remaining after four hours. To examine the half-lives of the SEB:Vβ8 interactions at 37° C., full dissociation rate curves were measured for clones G4-9 and G5-8 (FIG. 28C). The half-life of the SEB:G5-8 interaction at 37° C. was approximately 20 minutes.

Nine clones were sequenced (FIG. 23) and all but one clone was derived from the library that contained a single amino acid extension in CDR1 (CDR1+1 library). There was a strictly conserved tyrosine at position 28, a strong preference for serine or threonine at the inserted residue (27a), and a preference for aspartic acid at residue 30. These preferences support the idea that these mutations contribute to the enhanced binding and longer off-rates of SEB for the Vβ mutants. The only clone (G5-10) from the wild-type length CDR1 library contained amino acids with long and bulky side chains (Arg, Trp) that may act by compensating for the lack of a CDR1 extension.

### Affinity Maturation of Vβ8 by Yeast Display

To engineer high-affinity receptor antagonists for SEB, the mouse Vβ8.2 domain was cloned into the yeast display vector, pCT202 (FIG. 22A). A stabilized mutant of the Vβ8 (called mTCR15) that exhibited higher surface levels on yeast, was used as the starting template for mutagenesis; mTCR15 contains the stabilizing mutation G17E. From the crystal structure of Vβ8 in complex with SEB, it is known that 50% of all contacts between the molecules are located in the CDR2 loop of the Vβ, which was the first focus for mutagenesis (FIG. 22B). Two libraries that each spanned four codons of this region were generated (A50-53 and A54-57) by PCR with degenerate primers, and the products were inserted into the yeast display vector, yielding libraries of 1.8×10⁷ and 8.1×10⁶ individual transformants, respectively (see Table 5 for all library sizes). These library sizes should cover virtually all possible combinations of amino acids at the four positions (32 codons⁴=10⁶ possible combinations). The libraries were incubated with 650 nM biotinylated-SEB, followed by phycoerythrin-labeled streptavidin. Fluorescence-activated cell sorting was used to collect the top 0.5% of fluorescent cells from each library, for a total of four consecutive rounds of cell sorting and growth. Yeast cells selected from the fourth round of sorting were plated and individual clones were analyzed for binding to SEB.

TABLE 5
Yeast display library sizes.

| | Library | Approximate Library |
|---|---|---|
| Generation One | CDR2Δ50-53 | 1.8 × 10⁷ |
| | CDR2Δ54-57 | 8.1 × 10⁶ |
| Generation Two | G1-17Δ54-57 | 5.2 × 10⁶ |
| | G1-18Δ54-57 | 4.4 × 10⁶ |
| | G1-24Δ50-53 | 9 × 10⁶ |
| Generation Three | G2-3Δ47-50 | 1.5 × 10⁷ |
| | G2-5Δ47-50 | 1.8 × 10⁷ |
| | G2-8Δ47-50 | 1.3 × 10⁷ |
| | G2-9Δ47-50 | 1.1 × 10⁷ |
| Generation Four | Random Mutagenesis | 6 × 10⁶ |
| Generation Five | G4-9 CDR1 (26-30) | 1.9 × 10⁷ |
| | G4-9 CDR1 + 1 (27-30) | 1.9 × 10⁷ |
| | G4-9 CDR1 + 2 (27a-30) | 1.9 × 10⁷ |

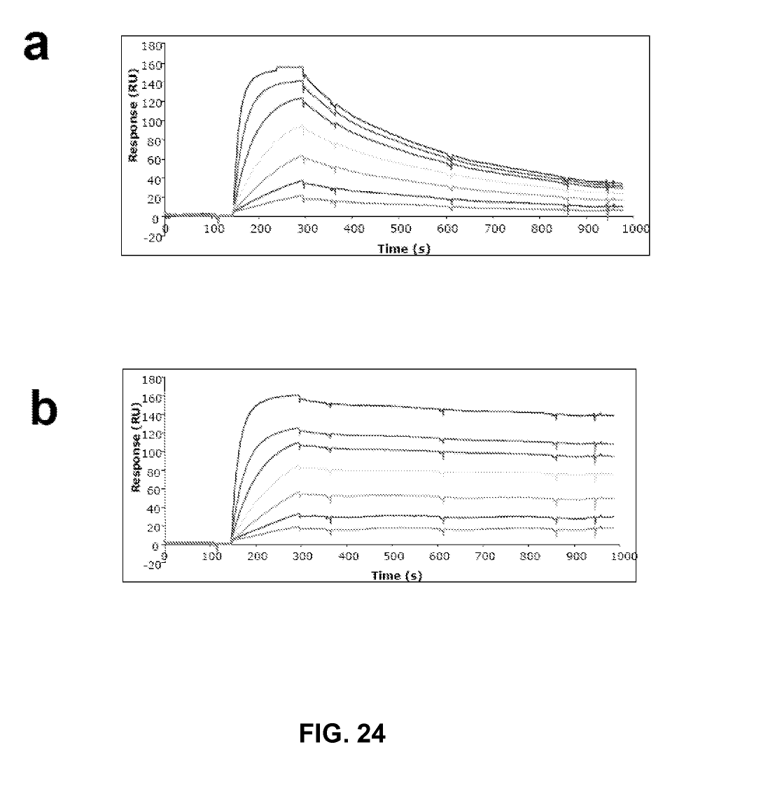

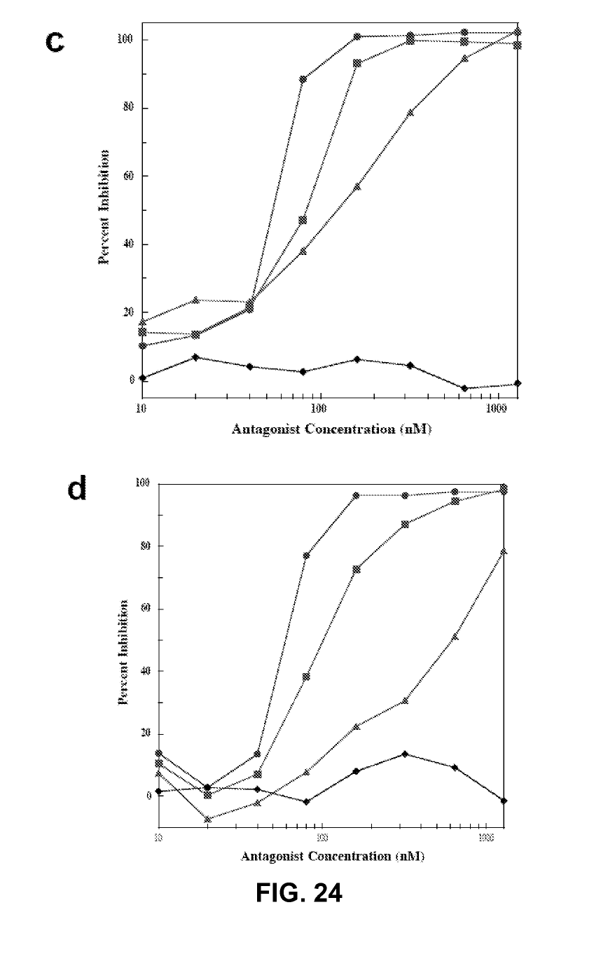

Five clones from each library of this first generation of mutagenesis and selection were screened for binding to SEB by flow cytometry and all were positive for binding to SEB, compared to wild-type Vβ which was undetectable at this concentration (FIG. 22C and FIG. 28A). Sequencing of the 10 clones revealed 3 unique sequences from each library (G1 clones, FIG. 24). All of the A50-53 clones retained the wild-type residue Gly51, consistent with our previous alanine scanning study that a G51A mutation had the largest impact on Vβ8 binding to the structurally similar toxin SEC3. A conserved A52I or A52V mutation was found in each clone. The A52V mutation has been shown in a structural study of an affinity matured Vβ8:SEC3 interaction to act by increasing the number of intermolecular contacts with Tyr90 of SEC3 (an identical residue with SEB) and by influencing the HV4 region. The other most conserved feature of the three Δ50-53 mutants was a substitution of positive-charged residues (H is or Arg) at Gly53. The side chain of this residue is in a position such that it could point directly into the cleft between the small and large domains of SEB (FIG. 22B). In addition, two of the mutants contained a single-site mutation (G42E) that was apparently the product of a PCR error. Like G17E, the G42E mutation is located distal to the SEB binding site and has been shown previously to be involved in enhancing surface display of the Vβ (FIG. 22B). The three unique clones derived from the Δ54-57 library also exhibited conserved features, the most notable being a S54H mutation. Since this residue is also positioned at the cleft between the SEB small and large domains, it may act like the positive-charged mutations at residue 53. Two of the three clones exhibited a presumed PCR-derived H47Y mutation. As described below, a similar mutation (H47F) was observed in subsequent engineering steps. It may provide some improvement in SEB binding as the side chain of His47 is within 4 Å of Phe177 of SEB.

Based on SEB-binding titrations of a first generation clone compared to the Vβ8 L2CM mutant that cross-reacted with SEB at an affinity of approximately 250 nM, the affinity of these lead clones appeared to be in the high nanomolar range (FIG. 28A and data not shown). To further affinity mature the Vβ8, second generation libraries were constructed using representative clones from the first generation as templates. Clones G1-17 and G1-18 were chosen from the Δ50-53 library, and clone G1-24 was chosen from the Δ54-57 library. To optimize the contacts in the CDR2 region, the adjacent CDR2 residues, 50-53 or 54-57, were randomized. Both libraries were sorted using 6 nM SEB and the top 0.5% of cells was collected for a total of four rounds of sorting. However, only the library derived from the Δ50-53 template yielded a positive population of yeast cells selected at this SEB concentration. Fifteen clones were screened for binding to 100 nM SEB, and each clone was improved compared to the G1-17 mutant and L2CM (FIG. 28B). Titrations of two of the clones suggested they had affinities in the low nanomolar range (FIG. 28B). Four unique sequences were observed among ten of the G2 clones that were sequenced (FIG. 23). All of the clones contained two mutations, S54N and T55V that must account for the increase in affinity since these were the only mutations in two of the clones.

Because residues on the N-terminal side of CDR2 are within contact distance of SEB, a third generation of affinity maturation focused on residues 47-50. Clones G2-3, G2-5, G2-8, and G2-9 were used as templates, the library was incubated with 100 μM SEB, and the top 0.5% of cells were collected through four rounds of sorting. A positive population of cells was isolated after four sorts, and 14 clones were analyzed for binding to 10 nM SEB. Eleven clones had improved binding to SEB (FIG. 27C) and the five clones with the highest fluorescence were sequenced (FIG. 23). All of the clones differed in sequence, but each contained a strictly conserved H47F mutation, and the wild type serine at position 49. As described above, the H47F mutation could be involved directly in SEB binding, whereas it appears that Ser49 probably acts indirectly by stabilizing the CDR2 loop.

Characterization of SEB-Binding Clones Isolated from Yeast Display

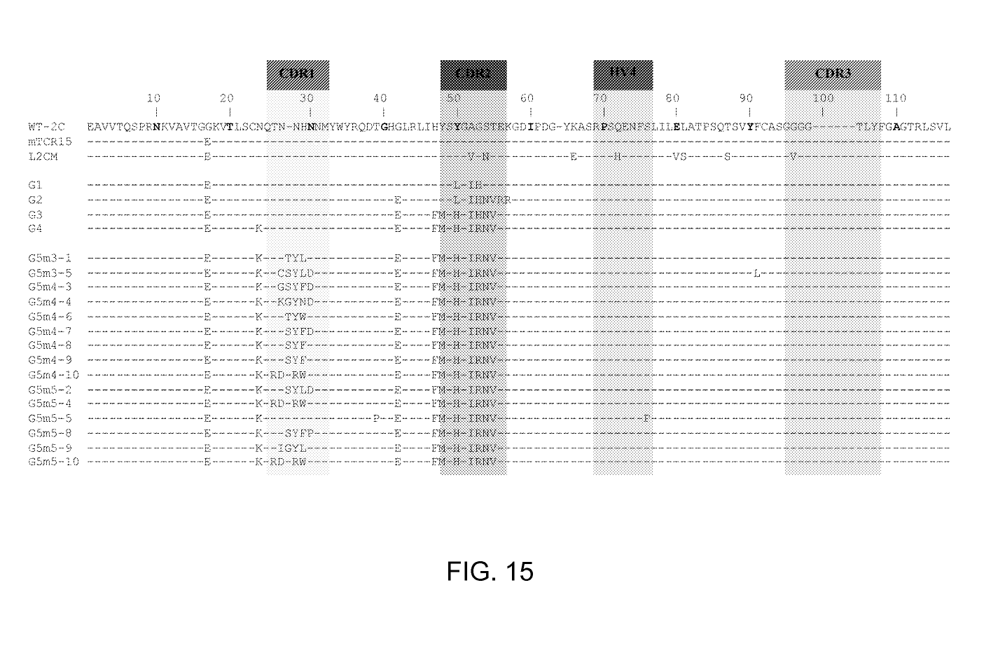

FIG. 15 shows the sequences of mVβ8.2 mutants isolated for binding to SEB. Clone mTCR15 is a stabilized mutant of mVβ8.2. LC2M was previously isolated for binding the closely related superantigen, SEC3, and has a low level of cross-reactivity for SEB. G2, G3, G4, and G5 refer to the generation of yeast display library from which the clone was isolated.

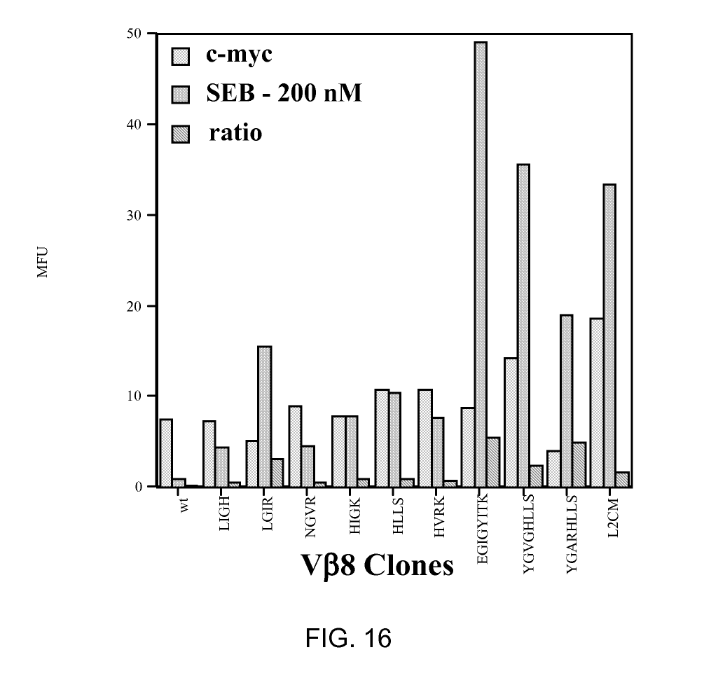

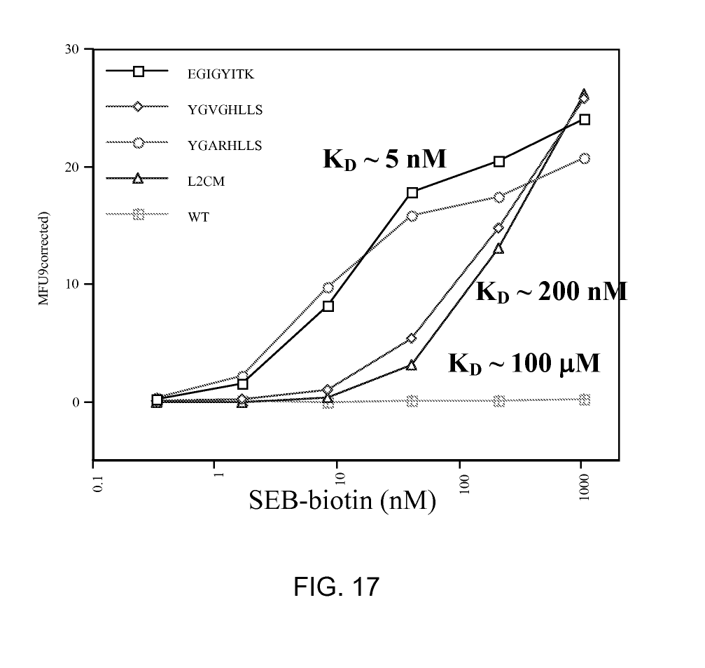

FIG. 16 shows binding of biotinylated SEB to yeast clones that express different Vβ8 mutants (where region CDR2 was mutated). FIG. 17 shows titrations of biotinylated SEB and yeast expressing Vβ8 mutants (CDR2) to determine affinities.

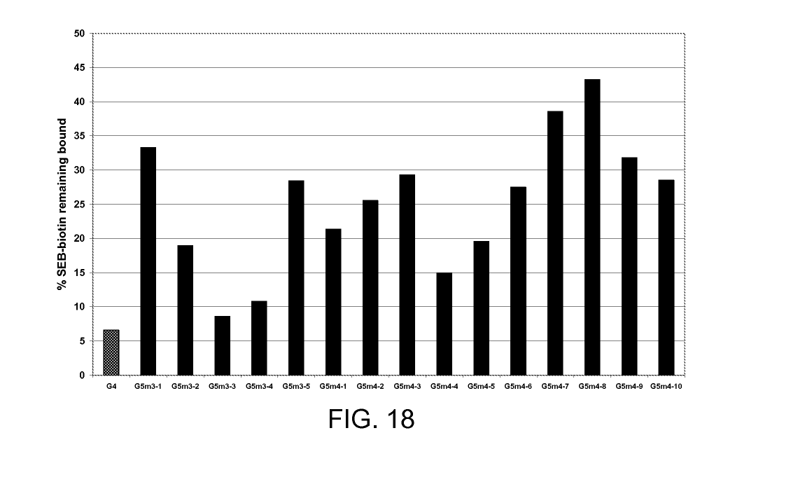

FIG. 18 shows binding of fifth generation clones to SEB. Clones were incubated with 5 nM biotinylated SEB for one hour under equilibrium conditions, and then incubated with a 10-fold molar excess of unlabeled SEB for 4 hours at 25° C. A sample was removed before the unlabeled SEB was added and placed on ice until the end of the experiment. Percent remaining bound was calculated as (MFU after 4 hours at 25° C./MFU at time zero)×100.

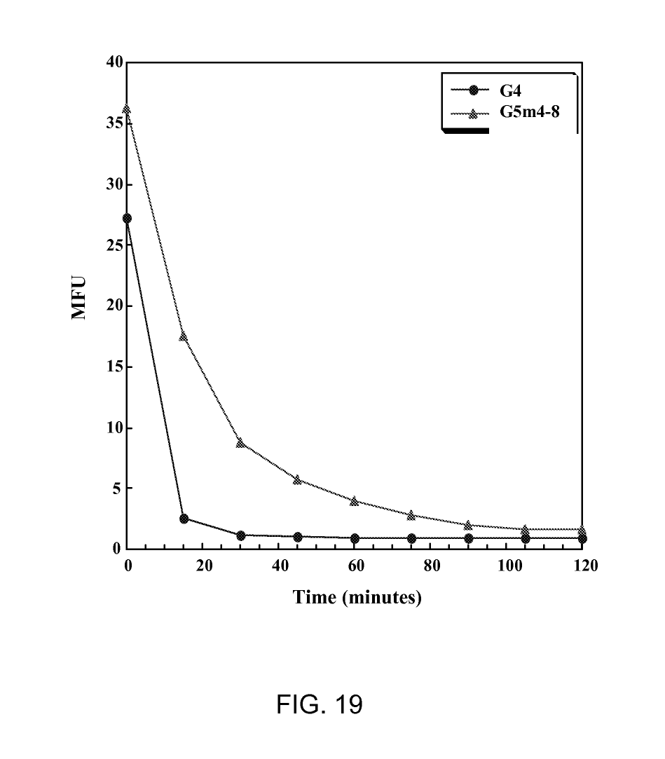

FIG. 19 shows off-rates of fourth generation (G4) and fifth generation (G5 m4-8) SEB-binding clones. The yeast displayed constructs were incubated with 5 nM biotinylated SEB for 1 h on ice, followed by incubation with a 10-fold molar excess of unlabeled SEB at 37° C. Aliquots were removed at the indicated timepoints and stored on ice until the end of the timecourse.

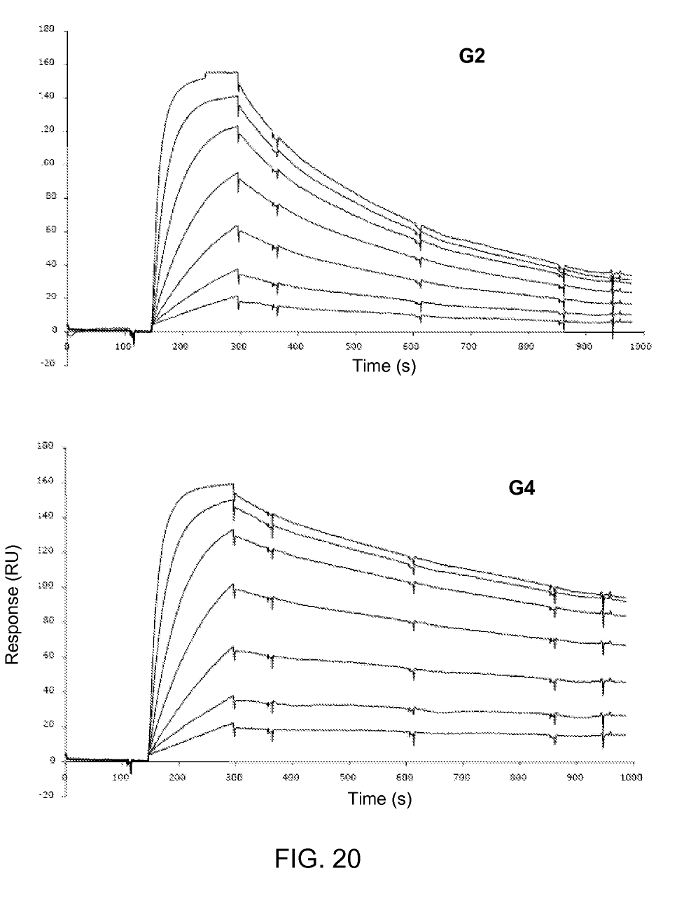

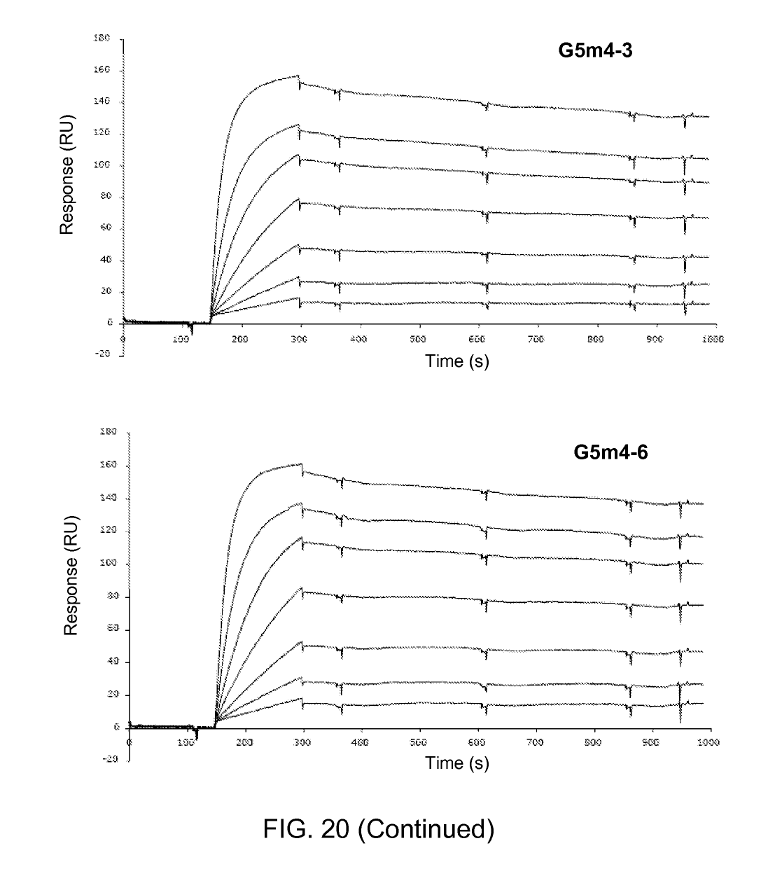

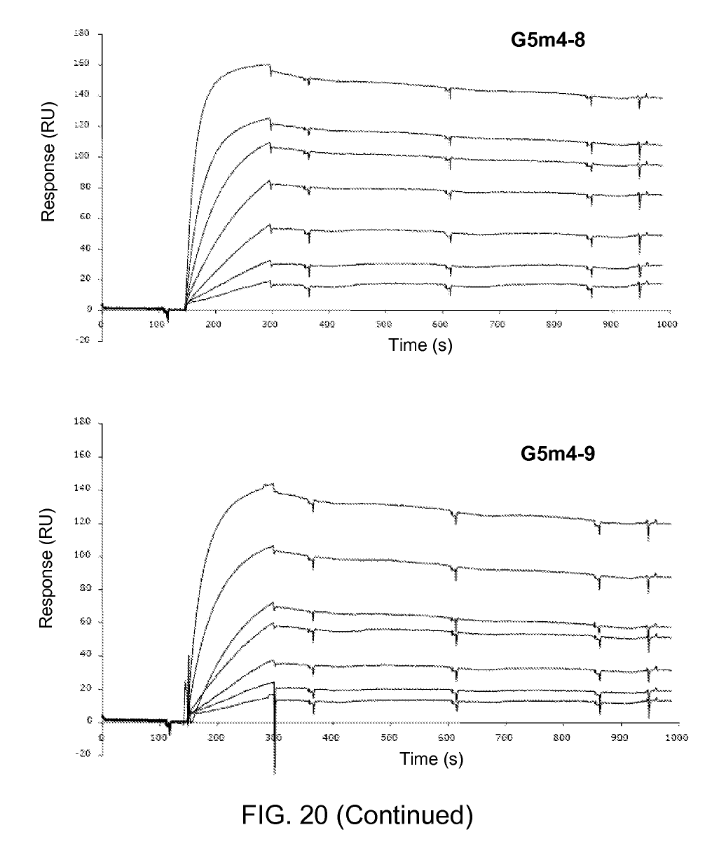

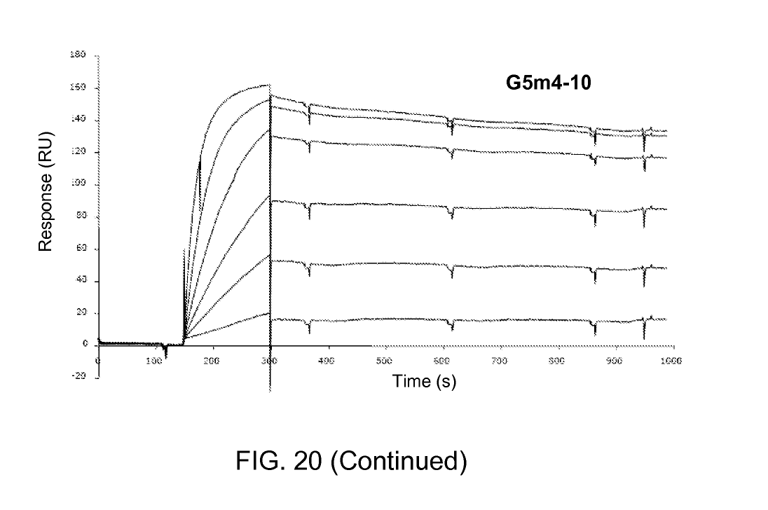

FIG. 20 shows surface plasmon resonance analysis of affinity matured mVb8.2 variants binding to SEB. SPR sensorgrams of 2-fold dilutions (20-0.3125 nM) of G2, G4, G5 m4-3, G5 m4-6, G5 m4-8, G5 m4-9 and G5 m4-10 variants binding to immobilized SEB (533 RU). Dilutions of the mVb8.2 variants are from top to bottom as follows: 20 nM; 10 nM; 5 nM; 2.5 nM; 1.25 nM; 0.625 nM; 0.3125 nM.

A previous study showed that successive generation of random mutants yielded improvements in a monoclonal antibody affinity from low nanomolar to femptomolar levels. Accordingly, a fourth generation library was made using error-prone PCR and the five sequenced clones from the third generation library (FIG. 23) as templates. The library was incubated with 100 μM SEB, and the top 0.5% of cells were collected through four rounds of sorting. A shift in the positive population of cells, compared to third generation clones, was observed. Nine clones were analyzed with 1 nM SEB by flow cytometry, and each of the clones showed significant binding at this SEB concentration (FIG. 27D). The five clones with the highest level of SEB binding were sequenced and a mutation was found in only a single position, N24S or N24K. Asn24 is located at the end of the CDR1 loop, and although it does not appear to be close enough to make contact with SEB, it appears to increase the level of surface display of the Vβ molecule (FIG. 28A) and hence it may play a role in Vβ stability.

While titration of the G4 mutants suggested the binding affinity was in the low nanomolar range (FIG. 28A), it was reasoned that the affinity might be further increased by generating ‘extension’ libraries in CDR1, and by using an off-rate based selection scheme. The CDR1 ‘extension’ engineering was based on the premise that SEB is only 7A from the CDR1 of Vβ8 and that SpeC contacts the CDR1 of human Vβ2.1, which contains an extra amino acid compared to Vβ8.2. To test the idea of using ‘extension’ libraries to reshape this interface, three yeast display libraries were made with different CDR1 lengths: ACDR1 (residues 26-30 randomized), CDR1+1 (residues 27-30 randomized, one amino acid inserted at position 27a), and CDR1+2 (residues 27a-30, with two amino acids inserted at positions 27a and b). Mutant G4-9 was used as template, and the three libraries were pooled at equal ratios prior to the first round of selection, using off-rate based sorting. The approach involved incubating the yeast with biotinylated SEB (5 nM) for one hour, followed by a two-hour incubation at 25° C. with a ten-fold molar excess of unlabeled SEB (two hours had been shown to yield >90% loss of bound SEB by clone G4-9). Yeast cells were sorted and a decreasing percentage of cells were collected through each of four rounds (1% to 0.25%). At the end of the third and fourth rounds, distinct positive populations of yeast cells were observed, and cells were plated to analyze individual colonies. Fifteen clones were screened based on a single-point off-rate of four-hour duration at 25° C. (FIG. 22D). All clones showed improvements over the fourth generation clone (G4-9), which had <10% bound ligand remaining after this incubation. In contrast, clone G5-8, had almost 50% bound SEB remaining after four hours at 25° C. To examine the half-lives of the SEB:Vβ8 interactions at 37° C., full dissociation rate curves were measured for clones G4-9 and G5-8 (FIG. 28C). The half-life of the SEB:Vβ8-G5-8 interaction at 37° C. was approximately 20 minutes.

A total of nine clones were sequenced (FIG. 23) and all but one clone was derived from the library that contained a single amino acid extension in CDR1 (CDR1+1 library). There was a strictly conserved tyrosine at position 28, a strong preference for serine or threonine at the inserted residue (27a), and a preference for aspartic acid at residue 30. These preferences support the idea that these mutations contribute to the enhanced binding and longer off-rates of SEB for these Vβ mutants. The only clone (G5-10) that was isolated from the wild-type length CDR1 library contained amino acids with long and bulky side chains (Arg, Trp) that may act by compensating for the lack of a CDR1 extension. In the crystal structures of both the SpeA-Vβ8.2 and SEC3-Vβ8.2 complexes, residue 28 of the CDR1 loop makes contact with the SAg.

Table 6 below shows the results of extending the CDR1 loop. The % bound represents the amount of SEB-biotin remaining bound after incubation at 25° C. for 4 h in the presence of 10× molar excess unlabeled SEB. “ND” indicates not determined.

TABLE 6
CDR1 Sequence

| | 26 | 27 | 27a | 28 | 29 | 30 | % Bound |
|---|---|---|---|---|---|---|---|
| WT | T | N | | N | H | N | ND |
| Gen3 | T | N | | N | H | N | 6 |
| Gen4m3 | T | G | S | Y | L | D | 29 |
| Gen4m6 | T | N | T | Y | W | N | 28 |
| Gen4m8 | T | N | S | Y | F | N | 43 |
| Gen4m9 | T | N | S | Y | F | D | 32 |
| Gen4m10 | R | D | | R | W | N | 28 |

### Expression and Binding Analyses of Soluble Vβ8 Mutants

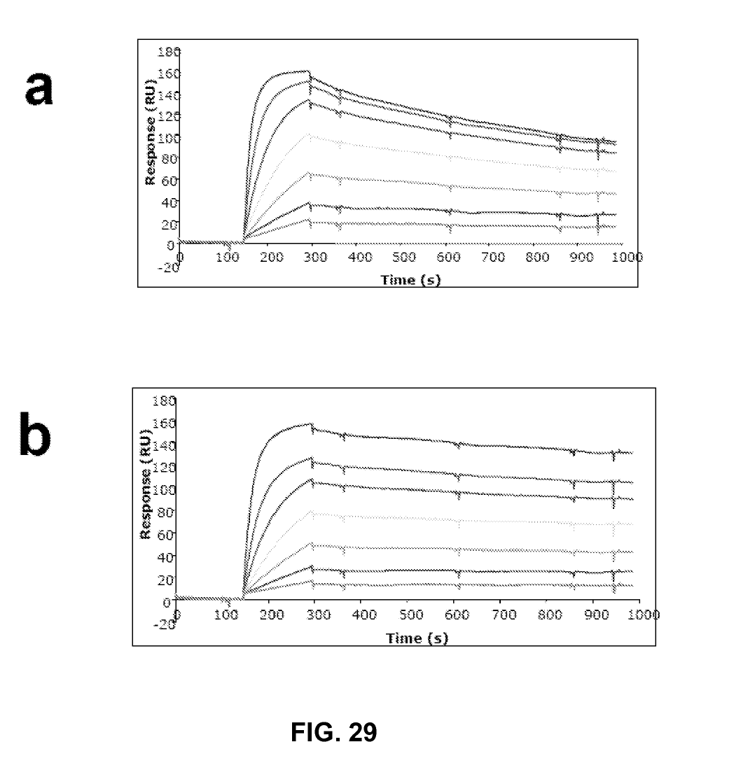

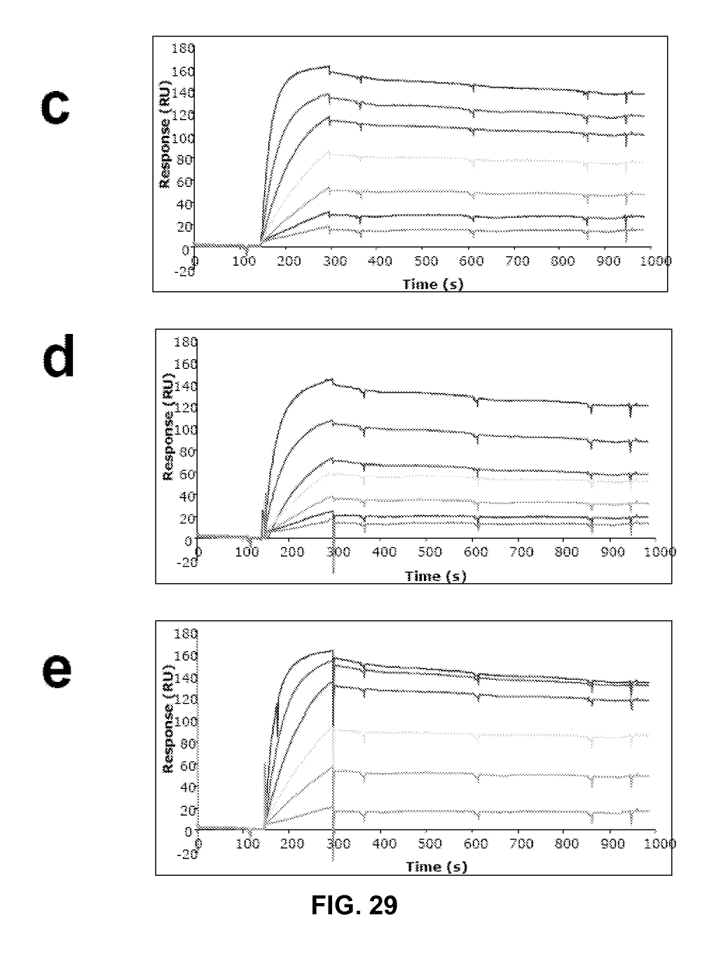

It has been shown that the incorporation of yeast-display stabilizing mutations improved expression and refolding of soluble TCR V regions in E. coli. To further characterize Vβ8 mutants and to examine their effectiveness as neutralizing agents, several clones were expressed in E. coli, and refolded from inclusion bodies. The binding affinity and kinetics of their interactions with SEB were measured using surface plasmon resonanace (SPR) (Table 7). Second-generation clone G2-5 was found to have an affinity of 650 pM (FIG. 24A), fourth-generation clone G4-9 had an affinity of 195 pM, and fifth-generation clones all had affinities of 48 to 100 pM (FIG. 24B, Table 1, and FIG. 29). The highest affinity mutant, G5-8, had a K_{D} value of 48 pM, a 3-million fold improvement in binding affinity for SEB compared to wild-type Vβ8.2 (K_{D} value=144 μM).

TABLE 7
SEB binding and in vitro inhibitory properties of Vβ8 affinity matured variants.

| | kₐ (M⁻¹s⁻¹) | k_{d} (s⁻¹) | K_{D} (M)² | t_{1/2} (min) | IC50 (nM)³ |
|---|---|---|---|---|---|
| WT TCR | | | 1.44 × 10⁻⁶ | | >2000 |
| (mTCR15) | | | | | |
| G2-5 | 3.81 ± 0.26 × 10⁶ | 2.48 ± 0.23 × 10⁻³ | 6.49 ± 0.16 × 10⁻¹⁰ | 4.6 | 860 ± 385 |
| G4-9 | 3.66 ± 0.29 × 10⁶ | 7.13 ± 0.44 × 10⁻⁴ | 1.95 ± 0.04 × 10⁻¹⁰ | 16.2 | 144 ± 49 |
| G5-4-3 | 2.99 ± 0.27 × 10⁶ | 2.47 ± 0.40 × 10⁻⁴ | 8.20 ± 1.24 × 10⁻¹¹ | 46.8 | ND⁴ |
| G5-4-6 | 3.16 ± 0.40 × 10⁶ | 1.91 ± 0.14 × 10⁻⁴ | 6.09 ± 0.42 × 10⁻¹¹ | 60.5 | ND |
| G5-4-8 | 3.44 ± 0.20 × 10⁶ | 1.64 ± 0.08 × 10⁻⁴ | 4.75 ± 0.12 × 10⁻¹¹ | 70.4 | 62 ± 15 |
| G5-4-9 | 2.50 ± 0.16 × 10⁶ | 2.32 ± 0.19 × 10⁻⁴ | 9.31 ± 0.50 × 10⁻¹¹ | 49.8 | ND |
| G5-4-10 | 3.04 ± 0.41 × 10⁶ | 2.09 ± 0.43 × 10⁻⁴ | 6.84 ± 0.87 × 10⁻¹¹ | 55.3 | ND |

¹Binding parameters derived from surface plasmon resonance (SPR) of three independent binding analyses for each bimolecular interaction, using global curve-fitting kinetic analysis and the BIAevaluation 4.1 software.
²Wild-type TCR mVβ8.2-Cβ was previously determined by SPR
³Based on the IC₅₀ values of three independent titrations in polyclonal T cell assays in the presence of 35 nM SEB
⁴Not determined

### In Vitro Neutralization of SEB by Soluble High-Affinity Vβ Regions

To explore whether successive generations of affinity-matured Vβ proteins also yielded improvements in neutralizing activity, in vitro T cell activation assays were performed. In these assays, the human class II-positive cell, Daudi, was loaded with ⁵¹Cr and incubated with SEB (35 nM) together with various concentrations of soluble Vβ antagonists. The Vβ8⁺ cytotoxic T cell clone 2C was used as effectors (FIG. 24C). Four soluble Vβ proteins were tested, including wild type Vβ8 (mTCR15), and representative Vβ proteins from three generations of the affinity maturation process: G2-5, G4-9, and G5-8. As expected, wild type Vβ8 (micromolar affinity) was completely ineffective at neutralizing the activity of SEB. In contrast, all three of the higher affinity Vβ antagonists inhibited SEB-mediated activity, with a clear correlation between neutralizing potential and affinity. 10₅₀ values were five- to ten-fold lower for G5-8 (K_{D}=48 pM) compared to G2-5 (K_{D}=650 pM).

In cases of TSS, SEB stimulates a polyclonal population of T cells that can express different Vβ regions. Therefore, the neutralizing potential of Vβ8 proteins was also examined using effector cells from heterogeneous T cell populations. SEB-reactive splenic T cells from BALB/c mice were induced in culture in the presence of SEB and used as effector cells together with Daudi target cells, SEB (35 nM), and soluble Vβ antagonists (FIG. 24D). The results were similar to those obtained with Vβ8⁺ CTL clone 2C, in that affinity-matured proteins exhibited neutralizing activities (IC₅₀ values) that were directly proportional to their binding affinities, with G5-8 exhibiting a 15-fold lower IC₅₀ value than G2-5 (Table 7). Thus, the Vβ antagonists are capable of neutralizing SEB-reactive T cells, regardless of the Vβ region that is expressed, and neutralization is enhanced by improvements in affinity.

### Example 5

### In Vivo Neutralization of SEB in Rabbit Models of Toxic Shock Syndrome

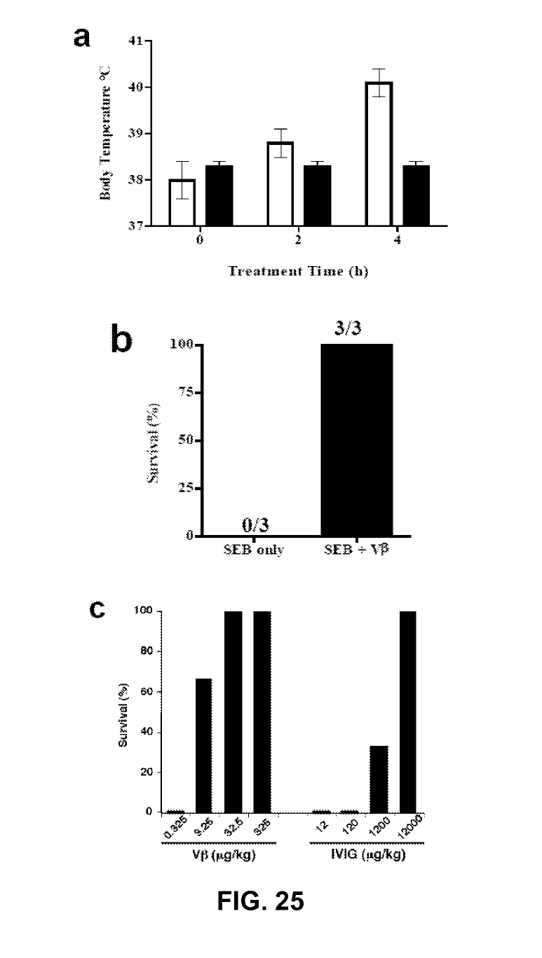

To determine if Vβ proteins were able to neutralize the activity of SEB in vivo, rabbit models of TSS and toxin-mediated lethality were examined. First, the Vβ was tested in an endotoxin-enhancement rabbit model. This model mimics the clinical situation in which patients with acute-phase TSS have detectable amounts of endotoxin in their sera. While the role of endotoxin in development of TSS in humans is not clear, exposure of rabbits to SAgs enhances their susceptibility to endotoxin shock by up to one million-fold. In this study, rabbits were injected with 5 μg/kg SEB, and fever response was monitored over the course of 4 hours. Rabbits invariably develop fevers within 4 hours and subsequent injection of Salmonella typhimurium LPS causes death in less than 12 hours. In the first experiment, 5 μg/kg/mL SEB was incubated with 500 μg/kg/mL of purified G5-8 Vβ (hereafter referred to only as Vβ) for one hour. Rabbits were then injected i.v. with the SEB/Vβ combination or 5 μg/kg/mL SEB alone (control), and fever response was monitored. Rabbits in the control group developed fevers (approximately 2° C. increase), whereas rabbits that received the SEB/Vβ combination exhibited no elevation in temperature (FIG. 25A). After four hours, each rabbit was injected i.v. with 0.15 μg/kg LPS, which is 100 times the LD₅₀ when pre-treated with 5 μg/kg SEB (the LD₅₀ of LPS alone is 500 μg/kg). All rabbits that were treated with SEB alone died, while all rabbits that were treated with SEB/Vβ survived with no adverse effects (FIG. 25B).

In an independent experiment, three rabbits that were injected with ten-times less neutralizing agent (50 μg/kg/mL Vβ) immediately after injection of SEB also showed no increase in temperature and survived (data not shown). Based on this result, the minimal Vβ dose that would be capable of protecting animals was assessed. In this experiment, four groups of rabbits (3 per group) were injected with SEB (5 μg/kg/mL) together with different amounts of the Vβ, from 0.325 to 325 μg/kg/mL. As shown in FIG. 25C, the Vβ neutralizing agent was completely protective at 32.5 and 325 μg/kg/mL and partially protective at 3.25 μg/kg/mL (two of the three rabbits survived). On a molar basis, this concentration of Vβ (Mol weight 12 kDa) is close to that of SEB used in the experiment (5 μg/kg/mL; Mol weight ˜28 kDa). This finding supports the notion that the high affinity of the G5-8 Vβ drives formation of the inactive complex (Vβ:SEB), even at low doses of the Vβ.

In order to compare the active concentration of Vβ with the current treatment of TSS involving human IVIG, various lots of human IVIG (ZLB Bioplasma AG lyophilized prep; IVEEGAM (Immuno AG) lyophilized prep; a Bayer IVIG liquid prep) were assayed for titers against SEB by ELISA. All three IVIG lots had titers of 640, better than the average titer of 80-160 found in most humans). In human TSS, IVIG is typically used at concentrations of 1000 to 2000 μg/kg. In accord with this, four groups of rabbits (3 per group) were injected with SEB (5 μg/kg/mL) together with different amounts of the IVIG preparation from ZLB Bioplasma, from 12 to 12000 μg/kg/mL. As shown in FIG. 25C, 12000 μg/kg/mL showed complete protection, 1200 μg/kg/mL showed partial protection (1 rabbit survived), and 120 and 12 μg/kg/mL showed no efficacy. The amount of IVIG and Vβ required to save one-half of the rabbits was estimated to be 6600 μg/kg/mL and 3 μg/ml/kg, respectively. Thus, there was approximately a 2200 fold difference in activity between human IVIG and the Vβ agent.

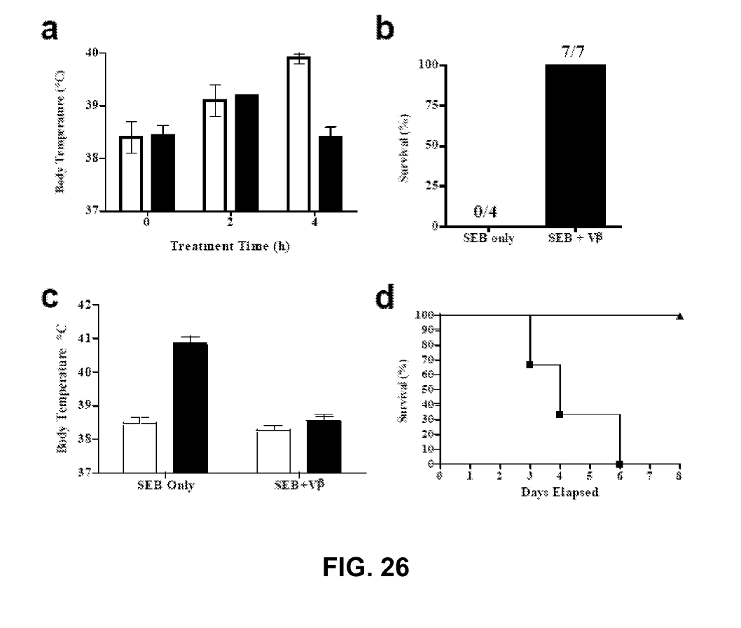

It was next examined whether rabbits with elevated temperatures due to SEB exposure could be rescued by treatment with Vβ. Rabbits were injected with 5 μg/kg SEB and two hours later, after rabbits had developed a fever, they were injected with 500 μg/kg of Vβ. Control rabbits continued to exhibit fevers at 4 hours, whereas the temperatures of rabbits that were treated with the Vβ returned to normal ranges (FIG. 26A). As in the experiment with combined treatment of SEB and Vβ, all rabbits that were treated with Vβ survived LPS exposure, whereas all control rabbits died (FIG. 26B). It was also examined if rabbits that were successfully treated with the Vβ protein would be susceptible to SEB a month later, and whether such rabbits could be successfully treated a second time (e.g. it is possible that induction of anti-SEB or anti-Vβ antibodies could have influenced a second exposure to SEB). In this experiment, four rabbits that were successfully treated were each administered SEB again. Two hours later, three of the four rabbits were given the Vβ. After LPS injections, all three treated rabbits survived, but the control rabbit (untreated after the second SEB exposure) died.

The final rabbit model involved a miniosmotic pump system for slow delivery of SEB. This system mimics the situation that might be encountered in a staphylococcal infection involving TSS. In this model, pumps containing 200 μg SEB/200 μL PBS were implanted in rabbits, and SEB was delivered at a rate of approximately 25 μg/day over 8 days. The experimental group received daily injections of 100 μg Vβ, beginning at the time that pumps were implanted. The temperatures of rabbits at time 0 and on day 2 showed that the control rabbits exhibited characteristic fevers, while the treated rabbits did not (FIG. 26C). All control rabbits died of TSS during the 8-day period, whereas all treated rabbits survived (FIG. 26D).

### Pharmacokinetic Study of Vβ in Rabbits

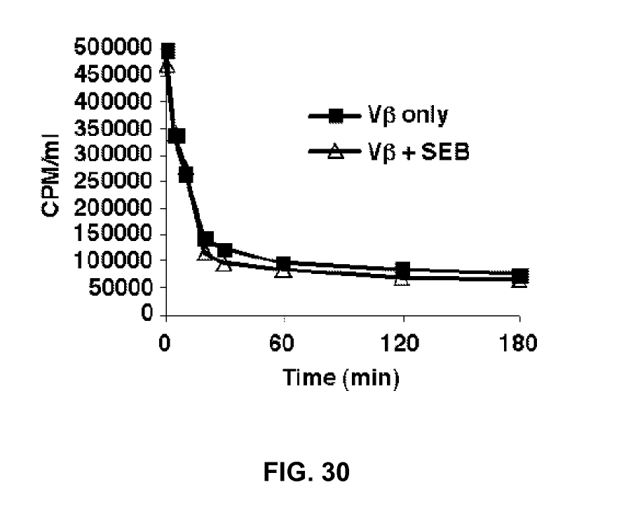

In order to gain insight into the in vivo action of the Vβ agents, a pharmacokinetic study was performed. Radiolabeled Vβ (¹²⁵I-Vβ) was injected into four rabbits, two without previous SEB treatment, and two that had an immediate prior injection with 200 μg of SEB and blood samples were taken from each rabbit. Analysis of the combined results (FIG. 30) using a two-phase exponential showed a t_{1/2} of the a redistribution phase of 7 minutes, and a t_{1/2} of the 13 clearance phase of 325 minutes. The presence of excess SEB did not have a significant effect on the serum lifetimes of the ¹²⁵I-Vβ^{˜}. These results appear to place the clearance properties of this Vβ domain between V_{H} or scFv fragments and Fc-bearing antibodies (scFv-C_{H}3, scFv-Fc or full IgG), which have been reported to have 13 phase t_{1/2} values of 20 to 30 minutes (V_{H}, scFv), 5 to 8 hours (scFv-C_{H}3), and several days to a week (scFv-Fc and IgG). It remains to be seen if Vβ domains in general will differ in their pharmocokinetic properties from different V_{H} domains.

To assess the in vivo distribution of the ¹²⁵I-Vβ, rabbits were euthanized and tissues were sampled for radioactivity three hours after injections (Table 8). The majority of counts were present in the urine, consistent with the small size of the protein (12 kDa). Among tissues sampled, the spleen showed the largest concentration of radiolabel, with a modest increase in the presence of SEB (170%). Kidney showed the most significant increase in localization of Vβ in the presence of SEB, with a 370% increase compared to Vβ in the absence of SEB. This result could be due to various effects, including the larger size of the Vβ:SEB complex (˜60 Å, compared to the free Vβ˜30 Å), which may alter the filtration properties of the molecule.

TABLE 8
Biodistribution of ¹²⁵I-labeled Vβ
in rabbits three hours after injection.

| | Vβ only | | Vβ and SEB | |
|---|---|---|---|---|
| | (% ID/gm)¹ | | (% ID/gm) | |
| Organ/Fluid | Rabbit #1 | Rabbit #2 | Rabbit #1 | Rabbit #2 |
| Liver | 0.184 | 0.164 | 0.173 | 0.177 |
| Kidney | 0.086 | 0.088 | 0.328 | 0.312 |
| Spleen | 0.567 | 0.540 | 0.889 | 1.003 |
| Thymus | 0.168 | 0.157 | 0.170 | 0.166 |
| Blood | 0.201 | 0.210 | 0.186 | 0.164 |
| Urine | 5.668 | 5.918 | 8.222 | 8.775 |

¹Percent injected dose per gram of tissue (or ml of blood and urine)

TABLE 9

| | SEQUENCE |
|---|---|
| SEQ. ID | DESIGNATION |
| NO. | IN FIG. 2 |
| 1 | WT |
| 2 | EP-5 |
| 3 | EP-6 |
| 4 | EP-7 |
| 5 | EP-8 |
| 6 | EP-9 |
| 7 | EP-11 |
| 8 | EP-12 |
| 9 | R3 |
| 10 | R9 |
| 11 | R15 |
| 12 | R17 |
| 13 | R18 |
| 14 | R21 |
| 15 | R24 |
| 16 | C4 |
| 17 | C8 |
| 18 | C10 |
| 19 | D9 |
| 20 | D10 |
| 21 | D19 |
| 22 | D20 |

TABLE 10

| | SEQUENCE |
|---|---|
| SEQ. ID | DESIGNATION |
| NO. | IN FIG. 15 |
| 23 | WT-2C |
| 24 | mTCR15 |
| 25 | L2CM |
| 26 | G1 |
| 27 | G2 |
| 28 | G3 |
| 29 | G4 |
| 30 | G5m3-1 |
| 31 | G5m3-5 |
| 32 | G5m4-3 |
| 33 | G5m4-4 |
| 34 | G5m4-6 |
| 35 | G5m4-7 |
| 36 | G5m4-8 |
| 37 | G5m4-9 |
| 38 | G5m4-10 |
| 39 | G5m5-2 |
| 40 | G5m5-4 |
| 41 | G5m5-5 |
| 42 | G5m5-8 |
| 43 | G5m5-9 |
| 44 | G5m5-10 |

TABLE 11

| | SEQUENCE |
|---|---|
| SEQ. ID | DESIGNATION |
| NO. | IN FIG. 23 |
| 45 | WT-2c |
| 46 | mTCR15 |
| 47 | G1-17 |
| 48 | G1-18 |
| 49 | G1-19 |
| 50 | G1-23 |
| 51 | G1-24 |
| 52 | G1-30 |
| 53 | G2-3 |
| 54 | G2-5 |
| 55 | G2-8 |
| 56 | G2-9 |
| 57 | G3-5 |
| 58 | G3-6 |
| 59 | G3-9 |
| 60 | G3-10 |
| 61 | G3-12 |
| 62 | G4-9 |
| 63 | G4-10 |
| 64 | G4-11 |
| 65 | G4-15 |
| 66 | G5-3 |
| 67 | G5-4 |
| 68 | G5-6 |
| 69 | G5-8 |
| 70 | G5-9 |
| 71 | G5-10 |
| 72 | G5-11 |
| 73 | G5-15 |

Presented below is the wild type Vβ2.1 sequence before stabilization.

GAVVSQHPSRVIAKSGTSVKIECRSLDFQATTMFWYRQFPKQSLMLMAT
SNEGSKATYEQGVEKDKFLINHASLTLSTLTVTSAHPEDSSFYICSALA
GSGSSTDTQYFGPGTRLTVL

Presented below is the wild type Vβ8.2 sequence designated WT-2c in FIG. 15 and FIG. 23.

EAVVTQSPRNKVAVTGGKVTLSCNQTN-NHNNMYWYRQDTGHGLRLIHY
SYGAGSTEKGDIPDG-YKASRPSQENFSLILELATPSQTSVYFCASGGG
G------TLYFGAGTRLSVL

### Discussion

Efforts to develop SEB neutralizing agents are particularly important because of SEB's potential as a biological weapon and because TSS may have even more of a clinical impact with the spread of methicillin resistant Staphylococcus aureus (MRSA). In fact, some strains of MRSA produce 10-100 times more exotoxin than their non-resistant counterparts, making them more likely to induce TSS. Potential neutralizing agents for SEB include monoclonal antibodies to SEB or human IVIG, which has been used in some severe cases of TSS. However, each of these approaches has significant drawbacks. Clinical use of anti-SEB monoclonal antibodies will require a dedicated program for generation, engineering, and pre-clinical testing of human antibodies, similar to that being conducted with antibodies to botulism toxin. On the other hand, human IVIG can exhibit variable success among different pools. For these reasons, a small, easily produced receptor-based therapeutic that directly blocks toxin action is presented here. This ˜12,000 dalton, Ig-like Vβ protein was engineered by sequential mutagenesis and selection to an affinity that is three million-fold higher than the wild-type receptor. The soluble receptor was able to prevent lethality in rabbit models of TSS (16/16 rabbits survived with treatment, 13/13 died without treatment—not including the experiment assessing Vβ versus IVIG dose efficacy). The protection occurred even when animals were treated with Vβ protein after exposure to toxin and after elevation in body temperature as is characteristic of TSS.

Previous studies with monoclonal antibodies to anthrax toxin and botulism toxin have shown that higher binding affinities are associated with improved inhibitory potential. The same relationship was observed here in SEB neutralization assays, even when comparing relatively high affinity Vβ proteins (e.g. K_{D} values of 650 to 48 pM). Accordingly, clinical effectiveness of soluble receptors can be optimized using the highest affinity agent available. A recent approach that compared soluble forms of two different receptors for anthrax toxin also showed that the higher affinity receptor was more effective. The approaches here of sequential engineering each contact region, and generating extensions in the CDR1, are generally useful in achieving such high-affinities. This is especially important when the contact surface involves a single Ig-like domain rather than the full antibody Fv, allowing maximal surface complementarity and interactions with ligand.

It is unclear if the relatively small size of these Vβ domains (12 KDa), or even their shorter serum lifetimes (β phase t_{1/2} of ˜325 minutes), may actually enhance their in vivo effectiveness compared to a full IgG molecule (150 KDa). It is possible that the ability of the smaller Vβ proteins to penetrate tissue more effectively than IgG may be useful, especially since the action of SAgs requires cell-to-cell interactions that occur in tissues. The pharmacokinetic studies performed with the Vβ suggest that its serum lifetime may be adequate to treat with excess agent on a daily basis, over a period of a few days. The efficacy, especially in comparison to human IVIG, suggests that the high-affinity of the agent may have allowed a dose that was near stoichiometric with SEB. In contrast, IVIG was required at high doses, perhaps because even in the highest titer preparations the level of natural antibodies to SEB are orders of magnitude lower than the single monospecific Vβ agent. There are some factors that should be optimized for the use of these therapeutic agents but the efficacy at low doses and the ability to express the proteins in E. coli provide additional evidence that Vβ treatments are feasible economically. Another advantage of their small size is that immunogenicity of the Vβ should be minimal. Immunogenicity associated with multiple injections of a protein should not pose a problem, since an individual who develops TSS may only do so once or twice in their lifetime, requiring a short course of therapeutic intervention without multiple chronic treatments common for monoclonal antibody therapy of autoimmune diseases or cancer. These factors are easily determined by one of ordinary skill in the art without undue experimentation.

The properties of these Vβ domains are not unlike camelid antibodies, or single V_{H} or V_{L} domains. These proteins have been characterized for their stability and solubility, leading to the development of human V_{H} domains that bind to selected antigens with high affinity. As shown here for engineered Vβ proteins, V_{H} proteins can also be expressed at high levels in E. coli, while full-length antibody production typically requires mammalian cell culture. Furthermore, the fact that the wild type Vβ:SEB interaction has even lower ‘starting’ affinity (K_{D}=144 μM) than many lead V_{H} proteins shows that the generation of Vβ domains that bind to other antigens is possible and that these are driven to very high-affinities

In the rabbit models that were tested here, successful treatment was observed in cases where the Vβ was administered after toxin had already induced elevated temperatures in animals. In addition, animals survived SEB delivered from miniosmotic pumps, when they were given daily injections of the neutralizing agent. It would not be difficult to provide such agents on a prophylactic or post-infection basis in a clinical setting by following routine procedures known to one having ordinary skill in the art. Clearly, staphylococcal or streptococcal infections might involve the presence of multiple toxins that may require neutralization. While the relative importance of SEB versus other toxins in the disease states is not clear, a single Vβ agent could neutralize more than one toxin. For example, G5-9, which binds to SEB with 93 pM affinity, was shown to bind to SEC3 with 2.5 nM affinity (data not shown). Although toxins such as TSST-1 have less structural similarity with SEB (than does SEC3), it is possible to generate picomolar binding affinity Vβ domains against TSST-1. One application is to rapidly detect the presence of specific toxins, and to match the toxins that are present with a neutralizing Vβ therapy.

### Methods

### Yeast Display Library Construction

The mVβ8.2 gene with the G17E stabilizing mutation was subcloned into the yeast display plasmid (pCT202) with an N-terminal HA tag and a C-terminal c-myc tag (FIG. 22A). Libraries of mutant Vβ TCR DNA were produced by site directed mutagenesis using overlapping degenerate primers (with NNS codons). After amplification, the PCR product was digested with Bsal and ligated into pCT202. The ligated product was digested with Dpnl to remove methylated template DNA and transformed into E. coli DH10B to amplify the plasmids. Intact plasmids were transformed by electroporation into the yeast strain EBY100. To create the fourth generation library of random mutants, the third generation templates (G3-5, 6, 9, 10, 12) were amplified using flanking primers with a method of error-prone PCR to give a 0.5% error rate. For the fourth and fifth generation templates, the PCR product was transformed along with NheI/XhoI digested pCT202 or NheI/BglII digested pCT302 into the yeast strain EBY100, which allows the PCR product to be inserted into the plasmid by homologous recombination. Transformants were grown on selective media for 48 hours. The estimated sizes of each the yeast display library is shown in Table 5.

### Fluorescence Activated Cell Sorting (FACS)

To induce protein expression, the yeast libraries were cultured for 24-48 h at 20° C. in medium containing galactose. For the first generation sorting, 5×10⁷ cells were incubated with 650 nM biotinylated SEB (Toxin Technology, Sarasota, Fla.) for one hour on ice. Cells were washed with PBS-0.5% BSA and stained with a 1:200 dilution of streptavidin-phycoerythrin (BD Pharmingen) in PBS-0.5% BSA and selected on a MoFlo high-speed cell sorter (Cytomation). The most fluorescent cells (0.5%) were collected, cultured overnight in selective media, and then induced in galactose-containing media for 20 h. This process was repeated three more times for a total of four rounds of sorting. After the fourth round of sorting, individual clones were obtained by plating on selective media. For the second generation library, 5×10⁷ cells were stained with 6 nM biotinylated SEB followed by a 1:200 dilution of streptavidin-PE. The most fluorescent cells (0.5%) were collected, for a total of four rounds of sorting. For the third generation library, 5×10⁷ cells were incubated with 100 μM biotinylated SEB, with a 1:200 dilution of streptavidin-PE. The most fluorescent cells (0.5%) were collected each round, over four rounds of sorting. For the fourth generation library, 5×10⁷ clones were stained using conditions identical to the third round of sorting. The fifth generation libraries were combined at equal ratios, and 1×10⁸ cells were incubated with 5 nM biotinylated SEB for one hour on ice. The cells were washed and then incubated with 50 nM unlabeled SEB for 2 hours in a 25° C. water bath (5×10⁷, 3×10⁷, 2×10⁷ cells were stained for the second, third and fourth sorts). Cells were stained with a 1:1000 dilution of streptavidin-PE. The most fluorescent cells (1%) were collected for the first round, followed by 0.5%, 0.5% and 0.25% for the second, third, and fourth sorts, respectively.

### Flow Cytometry of Isolated Mutants

Individual yeast clones were grown in glucose containing media at 30° C. and protein expression was induced by culturing in galactose containing media at 20° C. for 24-36 hours. Cells (4×10⁵) were incubated with various concentrations of biotinylated-SEB for one hour on ice. After washing, cells were incubated with a 1:500 dilution of streptavidin-PE. Fluorescence levels were measured on a Coulter Epics XL flow cytometer.

### Purification of Soluble Vβ Domains

All proteins used in the in vitro activity assays and in the rabbit experiments were expressed in BL21(DE3) E. coli using the pET28 expression vector (Novagen). The protein was refolded in vitro from inclusion bodies as described previously. Proteins were purified with Ni agarose resin (Qiagen, Valencia, Calif.) or by ion exchange chromatography (MonoS column, GE Healthcare, Piscataway, N.J.) followed by HPLC (BioCad Sprint) using a gel filtration column (Superdex 100 column, GE Healthcare, Piscataway, N.J.)). Proteins were dialyzed against PBS, pH 7.4 before use in tissue culture experiments or in animals.

### Surface Plasmon Resonance

SPR analysis of Vβ-SEB interactions was performed as described previously. Briefly, Vβ proteins were purified by an additional gel filtration chromatography step in HBS, pH 7.4 just prior to binding analysis on a Bicaore 3000 SPR instrument (Biacore, Piscataway, N.J.). SEB was immobilized by standard amine coupling to a CM5 sensor chip at a density of 500 response units (RU). An equivalent density of TSST-1, which exhibits no detectable binding to Vβ8 or its affinity-matured variants, was used as a control surface for all experiments. Serial dilutions of Vβ proteins were injected for up to 3 minutes at a flow rate of 25 ml/min and allowed to dissociate for up to 10 minutes prior to regeneration of the binding surfaces. The kinetic parameters for association and dissociation were determined using the BiaEvaluation 4.1 software (Biacore, Piscataway, N.J.).

### T Cell Assays

Daudi, a human lymphoma expressing class II MHC, but not class I, was maintained in RPMI 1640 supplemented with 10% FCS, 5 mM HEPES, 2 mM L-glutamine, 100 U penicillin, 0.1 mg/mL streptomycin, and 4×10⁻⁶M β-mercaptoethanol (KF media). 2C CTLs were expanded in culture until confluent by culturing in KF media supplemented with 10% rat concavalin A supernatant+5% α-methyl mannoside, and mitomycin C treated Balb/c splenocytes. Polyclonal CTLs were expanded from balb/c splenocytes by culturing at a density of 4×10⁶ cells per well in a 24-well plate for 72 hours in KF media, 10% rat concavalin A supernatant, 5% α-methyl mannoside, and 1 μg/mL SEB. Daudi cells were resuspended in 100 μCi ⁵¹Cr (MP Biomedicals) for one hour at 37° C. After washing, 10⁴ Cr-loaded Daudi cells were added in a volume of 504/well in 96-well U-bottom plates. SEB was added to a final concentration of 1 μg/mL (35 nM). Soluble Vβ protein was added at various concentrations in a volume of 50 μL. Plates were incubated at 37° C., 5% CO₂ for 30 min. 10⁵ CTLs were added per well in a volume of 50 μL. RPMI media was added to standardize well volumes to 200 μL. Plates were centrifuged 5 min at 800 rpm, and incubated at 37° C., 5% CO₂ for 4 hours. 80 μL of cell supernatant was removed after centrifugation of the plate for 5 min at 800 rpm, and ⁵¹Cr release was measured in a gamma counter. Percent inhibition was calculated as ((max cpm−experimental cpm)/(maximum cpm))×100. For all values, spontaneous release cpm were subtracted.

### Endotoxin Enhancement Model of Toxic Shock Syndrome

SAgs have been well-characterized to amplify the lethal effects of endotoxin through synergistic release of TNF-α. There is an inverse log:log relationship between dose SAg pretreatment and dose of endotoxin required to cause deaths of rabbits. In these studies, young adult rabbits (approximately 2 kg, both sexes) were conditioned to a pyrogen test apparatus, equipped with continuously in-place rectal thermocouples, for 3 hours the day before use and 1 hour the day of use. At the beginning of experimentation, the animals were injected intravenously with SEB (5 μg/kg/ml) in PBS (0.005M sodium phosphate, pH 7.2, 0.15M NaCl) and temperatures monitored hourly for 4 hours. At designated time points, intravenous injections of soluble antagonist and endotoxin (Salmonella typhimurium), 0.15 μg/kg/ml, were given. Animals were monitored for up to 48 hours for signs of TSS and death. Signs of TSS included fever, diarrhea, labored breathing, and conjunctival reddening.

IVIG preparations were generously provided by ZLB Bioplasma AG, Berne, Switzerland; Immuno AG, Vienna Austria (Oesterreichisches Institut fuer Haemoderivate G.m.b.H., IVEEGAM); and Bayer Healthcare, Leverkusen, Germany and used according to the manufacturers' recommendations. ELISA for determination of antibody titers against SEB were performed with use of Nunc-Immuno plates Maxisorp (Roskilde, Denmark). Plates were coated with 1 μg SEB, and serial 2-fold dilutions of IVIG preparations were made, beginning with a 1:10 dilution. Assays were developed with peroxidase conjugated goat antibodies against human IgG (Sigma-Aldrich, Inc. St. Louis, Mo.). Titers were determined as the reciprocal of the last dilution to give an absorbance at 490 nm wavelength of greater than the negative control.

For use in rabbits, Vβ and IVIG protein concentrations were quantified using the Bio-Rad protein assay (Bio-Rad Laboratories, Hercules, Calif.). The samples were diluted in sterile PBS for intravenous injection into marginal ear veins. Dose ranges for administration to rabbits were 0.325 to 325 μg/ml/kg for Vβ and 12 to 12,000 μg/ml/kg for human IVIG. Animals were injected with SEB (5 μg/kg/ml) and then 4 hours later endotoxin (0.15 μg/kg/ml) as above. Deaths were recorded over 48 hours. The LD₅₀ method of Reed and Muench was used to estimate the doses of Vβ and IVIG required for 50% protection of animals. All animal experimentation was performed according to guidelines of the University of Minnesota IACUC.

### Miniosmotic Pump Model of Toxic Shock Syndrome

The model of Parsonnet et al. was used to assess the ability of Vβ protein to inhibit TSS development during continuous SAg administration. Rabbits are highly sensitive to the TSS-inducing and lethal effects of SAgs when continuously released from subcutaneously implanted miniosmotic pumps (Alza, Palo Alto, Calif.). These pumps have been shown to release a constant amount of SAg to animals over the course of 8 days. An SEB dose of 200 μg is approximately 4 times the LD₅₀ by this model. Young adult rabbits (approximately 2 kg, either sex) were anesthetized with ketamine and xylazine and 1 cm incisions made on the left flanks. A subcutaneous pocket was made in each rabbit that was large enough to accommodate the miniosmotic pumps (0.5 cm×2 cm). Miniosmotic pumps were loaded with SAgs and implanted; the animals are then closed with three sutures and allowed to wake. The animals were returned to their cages and monitored for TSS symptoms and death over the course of 8 days. Fevers in this model occur maximally on day 2. Soluble Vβ was administered i.v. in PBS daily.

### Pharmacokinetic Studies

Radiolabeling of soluble Vβ with ¹²⁵I was performed by G. Brown, GE Healthcare, Woburn, Mass. with use of the lactoperoxidase method. The iodinated Vβ was determined to have a specific activity of 161 μCi/μg soluble Vβ, with 1.8% free iodide. By radioimmunoassay, at least 70% of the radiolabel was able to bind SEB immobilized on ELISA plates.

In pharmacokinetic studies, 4 rabbits were administered iodinated Vβ (35.48×10⁶ cpm in 1 ml of PBS containing 1% normal rabbit serum). Two rabbits received 200 μg SEB in 1 ml PBS intravenously immediately prior to receiving Vβ, and two rabbits received 1 ml of PBS prior to receiving Vβ. Blood samples (0.1 ml) were drawn from the marginal ear veins of each rabbit at 30 seconds and then 5, 10, 20, 30, 60, 120, and 180 minutes after injection. Radioactivity in blood samples was determined with use of a Perkin Elmer Wizard 1470 gamma counter (Shelton, Conn.). At the end of 180 minutes, each animal was sacrificed, and samples of various organs and urine were removed for determination of Vβ in tissues. At termination of the experiment, approximately 75% of the iodinated Vβ in the blood retained ability to bind to SEB immobilized onto ELISA plates (data not shown).

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

All patents and publications mentioned in the specification are indicative of the levels of skill of those skilled in the art to which the invention pertains. References cited herein are incorporated by reference herein in their entirety to indicate the state of the art, in some cases as of their filing date, and it is intended that this information can be employed herein, if needed, to exclude (for example, to disclaim) specific embodiments that are in the prior art. For example, when a compound is claimed, it should be understood that compounds known in the prior art, including certain compounds disclosed in the references disclosed herein (particularly in referenced patent documents), are not intended to be included in the claim.

When a Markush group or other grouping is used herein, all individual members of the group and all combinations and subcombinations possible of the group are intended to be individually included in the disclosure.

Every formulation or combination of components described or exemplified can be used to practice the invention, unless otherwise stated. Specific names of substances are intended to be exemplary, as it is known that one of ordinary skill in the art can name the same substances differently. When a compound is described herein such that a particular isomer or enantiomer of the compound is not specified, for example, in a formula or in a chemical name, that description is intended to include each isomers and enantiomer of the compound described individual or in any combination. One of ordinary skill in the art will appreciate that methods, superantigens, starting materials, and synthetic methods other than those specifically exemplified can be employed in the practice of the invention without resort to undue experimentation. All art-known functional equivalents, of any such methods, superantigens, starting materials, and synthetic methods are intended to be included in this invention. Whenever a range is given in the specification, for example, a temperature range, a time range, or a composition range, all intermediate ranges and subranges, as well as all individual values included in the ranges given are intended to be included in the disclosure.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising”, particularly in a description of components of a composition or in a description of elements of a device, is understood to encompass those compositions and methods consisting essentially of and consisting of the recited components or elements. The invention illustratively described herein suitably may be practiced in the absence of any element or elements, limitation or limitations which is not specifically disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims. Applicant does not wish to be bound by any theory presented herein.

In general the terms and phrases used herein have their art-recognized meaning, which can be found by reference to standard texts, journal references and contexts known to those skilled in the art. The definitions provided are to clarify their specific use in the context of the invention.

The exact formulation, route of administration and dosage can be chosen by the individual physician in view of the patient's condition (see e.g. Fingl et. al., in The Pharmacological Basis of Therapeutics, 1975, Ch. 1 p. 1).

It should be noted that the attending physician would know how to and when to terminate, interrupt, or adjust administration due to toxicity, or to organ dysfunctions. Conversely, the attending physician would also know to adjust treatment to higher levels if the clinical response were not adequate (precluding toxicity). The magnitude of an administered dose in the management of the disorder of interest will vary with the severity of the condition to be treated and to the route of administration. The severity of the condition may, for example, be evaluated, in part, by standard prognostic evaluation methods. Further, the dose and perhaps dose frequency, will also vary according to the age, body weight, and response of the individual patient. A program comparable to that discussed above also may be used in veterinary medicine.

Depending on the specific conditions being treated and the targeting method selected, such agents may be formulated and administered systemically or locally. Techniques for formulation and administration may be found in Alfonso and Gennaro (1995). Suitable routes may include, for example, oral, rectal, transdermal, vaginal, transmucosal, or intestinal administration; parenteral delivery, including intramuscular, subcutaneous, or intramedullary injections, as well as intrathecal, intravenous, or intraperitoneal injections.

For injection, the agents of the invention may be formulated in aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art.

Use of pharmaceutically acceptable carriers to formulate the compounds herein disclosed for the practice of the invention into dosages suitable for systemic administration is within the scope of the invention. With proper choice of carrier and suitable manufacturing practice, the compositions of the present invention, in particular those formulated as solutions, may be administered parenterally, such as by intravenous injection. Appropriate compounds can be formulated readily using pharmaceutically acceptable carriers well known in the art into dosages suitable for oral administration. Such carriers enable the compounds of the invention to be formulated as tablets, pills, capsules, liquids, gels, syrups, slurries, suspensions and the like, for oral ingestion by a patient to be treated.

Agents intended to be administered intracellularly may be administered using techniques well known to those of ordinary skill in the art. For example, such agents may be encapsulated into liposomes, then administered as described above. Liposomes are spherical lipid bilayers with aqueous interiors. All molecules present in an aqueous solution at the time of liposome formation are incorporated into the aqueous interior. The liposomal contents are both protected from the external microenvironment and, because liposomes fuse with cell membranes, are efficiently delivered into the cell cytoplasm. Additionally, due to their hydrophobicity, small organic molecules may be directly administered intracellularly.

Pharmaceutical compositions suitable for use in the present invention include compositions wherein the active ingredients are contained in an effective amount to achieve the intended purpose. Determination of the effective amounts is well within the capability of those skilled in the art, especially in light of the detailed disclosure provided herein.

In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions, including those formulated for delayed release or only to be released when the pharmaceutical reaches the small or large intestine.

The pharmaceutical compositions of the present invention may be manufactured in a manner that is itself known, e.g., by means of conventional mixing, dissolving, granulating, dragee-making, levitating, emulsifying, encapsulating, entrapping or lyophilizing processes.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

One skilled in the art would readily appreciate that the present invention is well adapted to carry out the objects and obtain the ends and advantages mentioned, as well as those inherent therein. The compositions and methods and accessory methods described herein as presently representative of preferred embodiments are exemplary and are not intended as limitations on the scope of the invention. Changes therein and other uses will occur to those skilled in the art, which are encompassed within the spirit of the invention, are defined by the scope of the claims.

Although the description herein contains many specificities, these should not be construed as limiting the scope of the invention, but as merely providing illustrations of some of the embodiments of the invention. Thus, additional embodiments are within the scope of the invention and within the following claims. Some references provided herein are incorporated by reference herein to provide details concerning additional starting materials, additional methods of synthesis, additional methods of analysis and additional uses of the invention.

### REFERENCES

1. Todd, J., Fishaut, M., Kapral, F. & Welch, T. (1978). Toxic-shock syndrome associated with phage-group-I Staphylococci. Lancet, 2, 1116-1118.
2. Schlievert, P. M., Shands, K. N., Dan, B. B., Schmid, G. P. & Nishimura, R. D. (1981). Identification and characterization of an exotoxin from Staphylococcus aureus associated with toxic-shock syndrome. J. Infect. Dis. 143, 509-516.
3. Bergdoll, M. S., Crass, B. A., Reiser, R. F., Robbins, R. N. & Davis, J. P. (1981). A new staphylococcal enterotoxin, enterotoxin F, associated with toxicshock-syndrome Staphylococcus aureus isolates. Lancet, 1, 1017-1021.
4. McCormick, J. K., Yarwood, J. M. & Schlievert, P. M. (2001). Toxic shock syndrome and bacterial superantigens: an update. Annu. Rev. Microbiol. 55, 77-104.
5. Kappler, J., Kotzin, B., Herron, L., Gelfand, E. W., Bigler, R. D., Boylston, A. et al. (1989). V beta-specific stimulation of human Tcells by staphylococcal toxins. Science, 244, 811-813.
6. Marrack, P. & Kappler, J. (1990). The staphylococcal entertoxins and their relatives. Science, 248, 705-711.
7. Prasad, G. S., Earhart, C. A., Murray, D. L., Novick, R. P., Schlievert, P. M. & Ohlendorf, D. H. (1993). Structure of toxic shock syndrome toxin 1. Biochemistry, 32, 13761-13766.
8. Acharya, K. R., Passalacqua, E. F., Jones, E. Y., Harlos, K., Stuart, D. I., Brehm, R. D. & Tranter, H. S. (1994). Structural basis of superantigen action inferred from crystal structure of toxic-shock syndrome toxin-1. Nature, 367, 94-97.
9. Sundberg, E. J., Li, Y. & Mariuzza, R. A. (2002). So many ways of getting in the way: diversity in the molecular architecture of superantigen-dependent T-cell signaling complexes. Curr. Opin. Immunol. 14, 36-44.
10. Choi, Y., Kotzin, B., Herron, L., Callahan, J., Marrack, P. & Kappler, J. (1989). Interaction of Staphylococcus aureus toxin “superantigens” with human Tcells. Proc. Natl. Acad. Acad. Sci. USA, 86, 8941-8945.
11. Choi, Y., Lafferty, J. A., Clements, J. R., Todd, J. K., Gelfand, E. W., Kappler, J. et al. (1990). Selective expansion of Tcells expressing V beta 2 in toxic shock syndrome. J. Exp. Med, 172, 981-984.
12. Sundberg, E. J., Li, H., Llera, A. S., McCormick, J. K., Tormo, J., Schlievert, P. M. et al. (2002). Structures of two streptococcal superantigens bound to TCR beta chains reveal diversity in the architecture of T cell signaling complexes. Structure (Camb), 10, 687-699.
13. McCormick, J. K., Tripp, T. J., Llera, A. S., Sundberg, E. J., Dinges, M. M., Mariuzza, R. A. & Schlievert, P. M. (2003). Functional analysis of the TCR binding domain of toxic shock syndrome toxin-1 predicts further diversity in MHC class II/superantigen/TCR ternary complexes. J. Immunol. 171, 1385-1392.
14. Boder, E. T. & Wittrup, K. D. (1997). Yeast surface display for screening combinatorial polypeptide libraries. Nature Biotech. 15, 553-557.
15. Kieke, M. C., Sundberg, E., Shusta, E. V., Mariuzza, R. A., Wittrup, K. D. & Kranz, D. M. (2001). High affinity T cell receptors from yeast display libraries block T cell activation by superantigens. J. Mol. Biol. 307, 1305-1315.
16. Kieke, M. C., Shusta, E. V., Boder, E. T., Teyton, L., Wittrup, K. D. & Kranz, D. M. (1999). Selection of functional T cell receptor mutants from a yeast surface-display library. Proc. Natl. Acad. Sci. USA, 96, 5651-5656.
17. Shusta, E. V., Holler, P. D., Kieke, M. C., Kranz, D. M. & Wittrup, K. D. (2000). Directed evolution of a stable scaffold for T-cell receptor engineering. Nature Biotechnol. 18, 754-759.
18. Shusta, E. V., Kieke, M. C., Parke, E., Kranz, D. M. & Wittrup, K. D. (1999). Yeast polypeptide fusion surface display levels predict thermal stability and soluble secretion efficiency. J. Mol. Biol. 292, 949-956.
19. Bentley, G. A., Boulot, G., Karjalainen, K. & Mariuzza, R. A. (1995). Crystal structure of the 13 chain of a Tcell antigen receptor. Science, 267, 1984-1987.
20. Garcia, K. C., Degano, M., Stanfield, R. L., Brunmark, A., Jackson, M. R., Peterson, P. A. et al. (1996). An αβ T cell receptor structure at 2.5 angstrom and its orientation in the TCR-MHC complex. Science, 274, 209-219.
21. Malchiodi, E. L., Eisenstein, E., Fields, B. A., Ohlendorf, D. H., Schlievert, P. M., Karjalainen, K. & Mariuzza, R. A. (1995). Superantigen binding to a T cell receptor β chain of known three-dimensional structure. J. Exp. Med. 182, 1833-1845.
22. Fields, B. A., Malchiodi, E. L., Li, H., Ysern, X., Stauffacher, C. V., Schlievert, P. M. et al. (1996). Crystal structure of a T-cell receptor □-chain complexed with a superantigen. Nature, 384, 188-192.
23. Li, H., Llera, A., Tsuchiya, D., Leder, L., Ysern, X., Schlievert, P. M. et al. (1998). Three-dimensional structure of the complex between a T cell receptor beta chain and the superantigen staphylococcal enterotoxin B. Immunity, 9, 807-816.
24. Li, H., Llera, A., Malchiodi, E. L. & Mariuzza, R. A. (1999). The structural basis of T cell activation by superantigens. Annu. Rev. Immunol. 17, 435-466.
25. Boder, E. T. & Wittrup, K. D. (1998). Optimal screening of surface-displayed polypeptide libraries. Biotechnol. Prog. 14, 55-62.
26. Boder, E. T., Midelfort, K. S. & Wittrup, K. D. (2000). Directed evolution of antibody fragments with monovalent femtomolar antigen-binding affinity. Proc. Natl. Acad. Sci. USA, 97, 10701-10705.
27. Chlewicki, L. K., Holler, P. D., Monti, B. C., Clutter, M. A. & Kranz, D. M. (2005). High-affinity, peptide specific Tcell receptors can be generated by mutations in CDR1, CDR2 or CDR3. J. Mol. Biol. 346, 223-239.
28. Chao, G., Cochran, J. R. & Wittrup, K. D. (2004). Fine epitope mapping of anti-epidermal growth factor receptor antibodies through random mutagenesis and yeast surface display. J. Mol. Biol. 342, 539-550.
29. Arden, B., Clark, S. P., Kabelitz, D. & Mak, T. W. (1995). Human T-cell receptor variable gene segment families. Immunogenetics, 42, 455-500.
30. Yang, J., Swaminathan, C. P., Huang, Y., Guan, R., Cho, S., Kieke, M. C. et al. (2003). Dissecting cooperative and additive binding energetics in the affinity maturation pathway of a protein-protein interface. J. Biol. Chem. 278, 50412-50421.
31. Arden, B., Clark, S., Kabelitz, D. & Mak, T. W. (1995). Mouse T-cell receptor variable gene segment families. Immunogenetics, 42, 501-530.
32. Burnett, J. C., Henchal, E. A., Schmaljohn, A. L. & Bavari, S. (2005). The evolving field of biodefence: therapeutic developments and diagnostics. Nature Rev. Drug Discov. 4, 281-297.
33. Holler, P. D., Holman, P. O., Shusta, E. V., O'Herrin, S., Wittrup, K. D. & Kranz, D. M. (2000). In vitro 320 T Cell Receptor-TSST-1 Interactions evolution of a T cell receptor with high affinity for peptide/MHC. Proc. Natl. Acad. Sci. USA, 97, 5387-5392.
34. Raymond, C. K., Pownder, T. A. & Sexson, S. L. (1999). General method for plasmid construction using homologous recombination. Biotechniques, 26, 134-138.
35. Starwalt, S. E., Masteller, E. L., Bluestone, J. A. & Kranz, D. M. (2003). Directed evolution of a single chain class II MHC product by yeast display. Protein Eng. 16, 147-156.
36. Blank, C. et al. Superantigen and endotoxin synergize in the induction of lethal shock. Eur J Immunol 27, 825-33 (1997).
37. Lowy, F. D. Staphylococcus aureus infections. N Engl J Med 339, 520-32 (1998).
38. Schlievert, P. M., Kotb, M. Y. & Stevens, D. L. Streptococcal superantigens: streptococcal toxic shock syndrome. in Effects of microbes on the immune system (eds. Cunningham, M. W. & Fujinami, R. S.) (Lippincott Williams & Wilkins, Philadelphia, 2000).
39. Horn, D. L. et al. What are the microbial components implicated in the pathogenesis of sepsis? Report on a symposium. Clin Infect Dis 31, 851-8 (2000).
40. Miller, J., Engelberg, S. & Broad, W. Germs: Biological Weapons and America's Secret War, (Simon & Schuster, New York, N.Y., 2001).
41. Crass, B. A. & Bergdoll, M. S. Involvement of staphylococcal enterotoxins in nonmenstrual toxic shock syndrome. J Clin Microbiol 23, 1138-9 (1986).
42. Lee, V. T., Chang, A. H. & Chow, A. W. Detection of staphylococcal enterotoxin B among toxic shock syndrome (TSS)- and non-TSS-associated Staphylococcus aureus isolates. J Infect Dis 166, 911-5 (1992).
43. Andrews, M. M., Parent, E. M., Barry, M. & Parsonnet, J. Recurrent nonmenstrual toxic shock syndrome: clinical manifestations, diagnosis, and treatment. Clin Infect Dis 32, 1470-9 (2001).
44. Schlievert, P. M., Tripp, T. J. & Peterson, M. L. Reemergence of staphylococcal toxic shock syndrome in Minneapolis-St. Paul, Minn., during the 2000-2003 surveillance period. J Clin Microbiol 42, 2875-6 (2004).
45. Jardetzky, T. S. et al. Three-dimensional structure of a human class II histocompatibility molecule complexed with superantigen. Nature 368, 711-718 (1994).
46. Kaul, R. et al. Intravenous immunoglobulin therapy for streptococcal toxic shock syndrome—a comparative observational study. The Canadian Streptococcal Study Group. Clin Infect Dis 28, 800-7 (1999).
47. LeClaire, R. D. & Bavari, S. Human antibodies to bacterial superantigens and their ability to inhibit T-cell activation and lethality. Antimicrob Agents Chemother 45, 460-3 (2001).
48. Hamad, A. R., Herman, A., Marrack, P. & Kappler, J. W. Monoclonal antibodies defining functional sites on the toxin superantigen staphylococcal enterotoxin B. J Exp Med 180, 615-21 (1994).
49. Pang, L. T., Kum, W. W. & Chow, A. W. Inhibition of staphylococcal enterotoxin B-induced lymphocyte proliferation and tumor necrosis factor alpha secretion by MAb5, an anti-toxic shock syndrome toxin 1 monoclonal antibody. Infect Immun 68, 3261-8 (2000).
50. Bradley, K. A., Mogridge, J., Mourez, M., Collier, R. J. & Young, J. A. Identification of the cellular receptor for anthrax toxin. Nature 414, 225-229 (2001).
51. Scobie, N. M. et al. A soluble receptor decoy protects rats against anthrax lethal toxin challenge. J Infect Dis 192, 1047-51 (2005).
52. Buonpane, R. A., Moza, B., Sundberg, E. J. & Kranz, D. M. Characterization of T cell receptors engineered for high affinity against toxic shock syndrome toxin-1. J Mol Biol 353, 308-21 (2005).
53. Garcia, K. C., Radu, C. G., Ho, J., Ober, R. J. & Ward, E. S. Kinetics and thermodynamics of T cell receptor-autoantigen interactions in murine experimental autoimmune encephalomyelitis. Proc Natl Acad Sci USA 98, 6818-23 (2001).
54. Weber, K. S., Donermeyer, D. L., Allen, P. M. & Kranz, D. M. Class II-restricted T cell receptor engineered in vitro for higher affinity retains peptide specificity and function. Proc Natl Acad Sci USA 102, 19033-8 (2005).
55. Stone, R. L. & Schlievert, P. M. Evidence for the involvement of endotoxin in toxic shock syndrome. J Infect Dis 155, 682-9 (1987).
56. Holtfreter, S. & Broker, B. M. Staphylococcal superantigens: do they play a role in sepsis? Arch Immunol Ther Exp (Warsz) 53, 13-27 (2005).
57. Schlievert, P. M. Enhancement of host susceptibility to lethal endotoxin shock by staphylococcal pyrogenic exotoxin type C. Infect Immun 36, 123-8 (1982).
58. Parsonnet, J. et al. Prevalence of toxic shock syndrome toxin 1-producing Staphylococcus aureus and the presence of antibodies to this superantigen in menstruating women. J Clin Microbiol 43, 4628-34 (2005).
59. Meissner, N. C., Schlievert, P. M. & Leung, D. Y. Mechanisms of immunoglobulin action: observations on Kawasaki syndrome and RSV prophylaxis. Immunol Rev 139, 109-23 (1994).
60. Kenanova, V. et al. Tailoring the pharmacokinetics and positron emission tomography imaging properties of anti-carcinoembryonic antigen single-chain Fv-Fc antibody fragments. Cancer Res 65, 622-31 (2005).
61. Olafsen, T. et al. Optimizing radiolabeled engineered anti-p185HER2 antibody fragments for in vivo imaging. Cancer Res 65, 5907-16 (2005).
62. Wu, A. M. & Senter, P. D. Arming antibodies: prospects and challenges for immunoconjugates. Nat Biotechnol 23, 1137-46 (2005).
63. Hoogenboom, H. R. Selecting and screening recombinant antibody libraries. Nat Biotechnol 23, 1105-16 (2005).
64. Cortez-Retamozo, V. et al. Efficient tumor targeting by single-domain antibody fragments of camels. Int J Cancer 98, 456-62 (2002).
65. Holliger, P. & Hudson, P. J. Engineered antibody fragments and the rise of single domains. Nat Biotechnol 23, 1126-36 (2005).
66. Nowakowski, A. et al. Potent neutralization of botulinum neurotoxin by recombinant oligoclonal antibody. Proc Natl Acad Sci USA 99, 11346-50 (2002).
67. Marks, J. D. Deciphering antibody properties that lead to potent botulinum neurotoxin neutralization. Movement Disorders 19 (S8), S101-S108 (2004).
68. Razai, A. et al. Molecular evolution of antibody affinity for sensitive detection of botulinum neurotoxin type A. J Mol Biol 351, 158-69 (2005).
69. Maynard, J. A. et al. Protection against anthrax toxin by recombinant antibody fragments correlates with antigen affinity. Nat Biotechnol 20, 597-601 (2002).
70. Rao, B. M., Lauffenburger, D. A. & Wittrup, K. D. Integrating cell-level kinetic modeling into the design of engineered protein therapeutics. Nat Biotechnol 23, 191-4 (2005).
71. Ward, E. S., Gussow, D., Griffiths, A. D., Jones, P. T. & Winter, G. Binding activities of a repertoire of single immunoglobulin variable domains secreted from Escherichia coli. Nature 341, 544-6 (1989).
72. Holt, L. J., Herring, C., Jespers, L. S., Woolven, B. P. & Tomlinson, I. M. Domain antibodies: proteins for therapy. Trends Biotechnol 21, 484-90 (2003).
73. Dinges, M. M. & Schlievert, P. M. Comparative analysis of lipopolysaccharide-induced tumor necrosis factor alpha activity in serum and lethality in mice and rabbits pretreated with the staphylococcal superantigen toxic shock syndrome toxin 1. Infect Immun 69, 7169-72 (2001).
74. Reed, L. J. & Muench, H. A simple method of estimating fifty percent endpoints. Am J Hyg 27, 493-497 (1938).
75. Parsonnet, J., Gillis, Z. A., Richter, A. G. & Pier, G. B. A rabbit model of toxic shock syndrome that uses a constant, subcutaneous infusion of toxic shock syndrome toxin 1. Infect Immun 55, 1070-6 (1987).
76. Roggiani, M., Stoehr, J. A., Leonard, B. A. & Schlievert, P. M. Analysis of toxicity of streptococcal pyrogenic exotoxin A mutants. Infect Immun 65, 2868-75 (1997).

We claim:

1. A method for making a stabilized T cell receptor variable region, comprising:
a) cloning the T cell receptor variable region gene in a yeast display vector;
b) mutagenizing the T cell receptor variable region to generate a library of mutants;
c) selecting the mutants which have the highest binding affinity to a ligand.

2. The method of claim 1, wherein the T cell receptor variable region is selected from the group consisting of Vα, Vβ, Vγ, and Vδ.

3. The method of claim 2, wherein the T cell receptor variable region is a human Vβ.

4. The method of claim 1, wherein the ligand is an antibody for the T cell receptor variable region.

5. The method of claim 1, wherein the T cell receptor is hVβ2 and the ligand is TSST-1.

6. The method of claim 1, wherein the T cell receptor is mVβ8 and the ligand is SEB.

7. The method of claim 1, further comprising repeating steps b) and c).

8. A stabilized T cell receptor variable domain comprising:
a T cell receptor variable region which contains one or more mutations wherein the stabilized T cell receptor variable domain binds with greater affinity to a ligand than wild type.

9. The stabilized T cell receptor variable domain of claim 8, wherein the variable domain is hVβ.

10. The stabilized T cell receptor variable domain of claim 9, wherein the variable domain contains at least one mutation selected from the group consisting of: S88G, R10M, A13V, L72P, and R113Q.

11. The stabilized T cell receptor variable domain of claim 8, wherein the variable domain is mVβ8, and the variable domain contains the mutation G17E and optionally one or more mutations selected from the group consisting of: N24K, G42E, H47F, Y48M, Y50H, A52I, G53R, S54N, and T55V.

12. A method for using stabilized T cell receptor variable region to select proteins that bind to a ligand with higher affinity than wild type comprising:
providing a stabilized T cell receptor variable region;
mutating the stabilized T cell receptor variable region to create a variegated population of mutants;
contacting the variegated population of mutants with a ligand;
selecting those mutants which bind to the stabilized T cell receptor variable region with higher affinity than wild type.

13. The method of claim 12, wherein the ligand is a superantigen.

14. The method of claim 12, wherein the mutant and ligand bind with an equilibrium binding constant K_{D}<1 μM.

15. The method of claim 14, wherein the mutant and ligand bind with an equilibrium binding constant K_{D}<100 nM.

16. A soluble mutant T cell receptor (TCR) variable region having higher affinity than the wild type T cell receptor for a bacterial superantigen, wherein said T cell receptor variable region is a mutant T cell receptor having one or more mutations in the TCR variable beta region.

17. The variable region of claim 16, wherein the variable region exhibits an equilibrium binding constant K_{D} for the bacterial superantigen of between about 10⁻⁸M and 10⁻¹²M.

18. The variable region of claim 16, wherein the variable region has one or more mutations in a CDR.

19. The variable region of claim 16, wherein the variable region has one or more mutations in a FR region.

20. The variable region of claim 16, wherein the bacterial superantigen is toxic shock syndrome toxin-1.

21. The variable region of claim 20, wherein the variable region has one or more mutations in the human Vβ2 region.

22. The variable region of claim 21, wherein the variable region has one or more mutations in the Vβ2.1 region.

23. The variable region of claim 20, wherein the variable region has one or more mutations in CDR2.

24. The variable region of claim 16, wherein the bacterial superantigen is staphylococcal enterotoxin B.

25. The variable region of claim 24, wherein the variable region has one or more mutations in the mouse Vβ8 domain.

26. The variable region of claim 24, wherein the variable region has one or more mutations in the Vβ8.2 domain.

27. The variable region of claim 16, wherein the mutant is selected from the group consisting of SEQ. ID Nos. 16-22; 30-44 and 66-73.

28. A method for treating staphylococcus infection in a mammal, the method comprising:
providing a high affinity mutant TCR variable region having one or more mutations in the TCR variable beta region, which TCR variable region binds to the superantigen with higher affinity than wild type TCR, wherein the high affinity TCR variable region interferes with the binding of the superantigen to the MHC class II molecules and T cell receptors of the mammal.

29. A method of treating a disease state in a mammal caused by a bacterial superantigen comprising:
administering an effective amount of a high affinity mutant of a T cell receptor variable region to a mammal.

30. The method of claim 29, wherein the disease is selected from the group consisting of: pneumonia, mastitis, phlebitis, meningitis, urinary tract infections; osteomyelitis, endocarditis, nosocomial infection, staphylococcal food poisoning and toxic shock syndrome.

31. The method of claim 29, wherein the high affinity mutant is selected from the group consisting of SEQ. ID Nos. 16-22; 30-44 and 66-73.

32. The method of claim 29, wherein the variable region is a variable beta region.

33. A therapeutic composition comprising a stabilized T cell receptor variable region and optional pharmaceutical additives.


SEQUENCE LISTING (73 sequences)

SEQ ID NO: 1 - protein, 118 aa, Homo sapiens
GAVVSQHPSRVIAKSGTSVKIECRSLDFQATTMFWYRQFPKQSLMLMATSNEGSKATYEQGVEKDKFLINHASLTLSTLTVTSAHPEDSSFYICSALAGSGSSTDTQYFGPGTRLTVL

SEQ ID NO: 2 - protein, 118 aa, Homo sapiens
GAVVSQHPSRVIAKSGTSVKIECRPLDFQATTMFWYRQFPKQSLMLMATSNEGSKATYEQGVEKDKFLINHASLTLSTLTVTSAHPEDSGFYICSALAGSGSSTDTQYFGPGTRLTVL

SEQ ID NO: 3 - protein, 118 aa, Homo sapiens
GAVVSQHPSRVIAKSGTSVKIECRTLDFQATTMFWYRQFPKQSLMLMATSNEGSKATYEQGVEKDKFLINHASLTLSTLTVTSAHPEDSGFYICSALAGSGSSTDTQYFGPGTRLTVL

SEQ ID NO: 4 - protein, 118 aa, Homo sapiens
GAVVSQHPSMVIAKSGTSVKIECRSLDFQATTMFWYRQFPKQSLMLMATSNEGSKATYEQGVEKDKFLINHASLTLSTLTVTSAHPEDSGFYICSALAGSGSSTDTQYFGPGTRLTVL

SEQ ID NO: 5 - protein, 118 aa, Homo sapiens
GAVVSQHPSRVIVKSGTSVKIECRSLDFQATTMFWYRQFPKQSLMLMATSNEGSKATYEQGVEKDKFLINHASLTLSTLTVTSAHPEDSGFYICSALAGSGSSTDTQYFGPGTRLTVL

SEQ ID NO: 6 - protein, 118 aa, Homo sapiens
GAVVSQHPSRVIAKSGTSVKIECRSLDFQATTMFWYRQFPKQSLMLMATSNEGSKATYEQGVEKDKFLINHASLTLSTLTVTSAHPEDSGFYICSALAGSGSSTDTQYFGPGTQLTVL

SEQ ID NO: 7 - protein, 118 aa, Homo sapiens
GAVVSQHPSRVIAKSGTSVKIECRSLDFQATTMFWYRQFPKQSLMLMATSNVGSKATYEQGVEKDKFLINHASPTLSTLTVTSAHPEDSSFYICSALAGSGSSTDTQYFGPGTRLTVL

SEQ ID NO: 8 - protein, 118 aa, Homo sapiens
GAVVSQHPSRVIAKSGTSVKIECRSLDFQATTMFWYRQFPKQSLMLMATSNEGSKATYEQGVEKDKFLINHASPTLSTLTVTSAHPEDSGFYVCSALAGSGSSTDTQYFGPGTRLTVL

SEQ ID NO: 9 - protein, 118 aa, Homo sapiens
GAVVSQHPSRVIAKSGTSVKIECRSLDFQATTMFWYRQFPKQSLMLMATSHLDMHATYEQGVEKDKFLINHASPTLSTLTVTSAHPEDSGFYICSALAGSGSSTDTQYFGPGTQLTVL

SEQ ID NO: 10 - protein, 118 aa, Homo sapiens
GAVVSQHPSMVIVKSGTSVKIECRSLDFQATTMFWYRQFPKQSLMLMATSRIDFHATYEQGVEKDKFLINHASPTLSTLTVTSAHPEDSGFYICSALAGSGSSTDTQYFGPGTQLTVL

SEQ ID NO: 11 - protein, 118 aa, Homo sapiens
GAVVSQHPSMVIAKSGTSVKIECRSLDFQATTMFWYRQFPKQSLMLMATSRMDYHATYEQGVEKDKFLINHASPTLSTLTVTSAHPEDSGFYICSALAGSGSSTDTQYFGPGTRLTVL

SEQ ID NO: 12 - protein, 118 aa, Homo sapiens
GAVVSQHPSRVIVKSGTSVKIECRSLDFQATTMFWYRQFPKQSLMLMATSRLWDSATYEQGVVKDKFLINHASPTLSTLTVTSAHPEDSGFYICSALAGSGSSTDTQYFGPGTQLTVL

SEQ ID NO: 13 - protein, 118 aa, Homo sapiens
GAVVSQHPSRVIVKSGTSVKIECRTLDFQATTMFWYRQFPKQSLMLMATSHQGFNAIYEQGVEKDKFLINHASPTLSTLTVTSAHPEDSGFYVCSALAGSGSSTDTQYFGPGTRLTVL

SEQ ID NO: 14 - protein, 118 aa, Homo sapiens
GAVVSQHPSRVIVKSGTSVKIECRSLDFQATTMFWYRQFPKQSLMLMATSRFGYHATYEQGVEKDKFLINHASPTLSTLTVTSAHPEDSGFYVCSALAGSGSSTDTQYFGPGTRLTVL

SEQ ID NO: 15 - protein, 118 aa, Homo sapiens
GAVVSQHPSRVIVKSGTSVKIECRSLDFQATTMFWYRQFPKQSLMLMATSRPGFHATYEQGVEKDKFLINHASPTLSTLTVTSAHPEDSGFYVCSALAGSGSSTDTQYFGPGTRLTVL

SEQ ID NO: 16 - protein, 118 aa, Homo sapiens
GAVVSQHPSRVIVKSGTSVKIECRSLERGSATMFWYRQFPKQSLMLMATSHQGFNAIYEQGVVKDKFLINHASPTLSTLTVTSAHPEDSGFYICSALAGSGSSTDTQYFGPGTQLTVL

SEQ ID NO: 17 - protein, 118 aa, Homo sapiens
GAVVSQHPSMVIVKSGTSVKIECRSLNFFSGTMFWYRQFPKQSLMLMATSHQGFNAIYEQGVVKDKFLINHASPTLSTLTVTSAHPEDSGFYICSALAGSGSSTDTQYFGPGTQLTVL

SEQ ID NO: 18 - protein, 118 aa, Homo sapiens
GAVVSQHPSMVIVKSGTSVKIECRSLDFQATTMFWYRQFPKQSLMLMATSRIDFHATYEQGVVKDKFLINHASPTLSTLTVTSAHPEDSGFYVCSALAGSGSSTDTQYFGPGTQLTVL

SEQ ID NO: 19 - protein, 118 aa, Homo sapiens
GAVVSQHPSMVIVKSGTSVKIECRSLGTPFDTMFWYRQFPKQSLMLMATSHQGFNAIYEQGVVKDKFLINHASPTLSTLTVTSAHPEDSGFYICSALAGSGSSTDTQYFGPGTQLTVL

SEQ ID NO: 20 - protein, 118 aa, Homo sapiens
GAVVSQHPSMVIVKSGTSVKIECRSLDTNIHTMFWYRQFPKQSLMLMATSHQGFNAIYEQGVVKDKFLINHASPTLSTLTVTSAHPEDSGFYVCSALAGSGSSTDTQYFGPGTQLTVL

SEQ ID NO: 21 - protein, 118 aa, Homo sapiens
GAVVSQHPSMVIVKSGTSVKIECRSLDGFSGTMFWYRQFPKQSLMLMATSHQGFNAIYEQGVVKDKFLINHASPTLSTLTVTSAHPEDSGFYICSALAGSGSSTDTQYFGPGTQLTVL

SEQ ID NO: 22 - protein, 118 aa, Homo sapiens
GAVVSQHPSRVIVKSGTSVKIECRSLNVAGSTMFWYRQFPKQSLMLMATSHQGFNAIYEQGVVKDKFLINHASPTLSTLTVTSAHPEDSGFYICSALAGSGSSTDTQYFGPGTRLTVL

SEQ ID NO: 23 - protein, 110 aa, Mus musculus
EAVVTQSPRNKVAVTGGKVTLSCNQTNNHNNMYWYRQDTGHGLRLIHYSYGAGSTEKGDIPDGYKASRPSQENFSLILELATPSQTSVYFCASGGGGTLYFGAGTRLSVL

SEQ ID NO: 24 - protein, 110 aa, Mus musculus
EAVVTQSPRNKVAVTGEKVTLSCNQTNNHNNMYWYRQDTGHGLRLIHYSYGAGSTEKGDIPDGYKASRPSQENFSLILELATPSQTSVYFCASGGGGTLYFGAGTRLSVL

SEQ ID NO: 25 - protein, 110 aa, Mus musculus
EAVVTQSPRNKVAVTGEKVTLSCNQTNNHNNMYWYRQDTGHGLRLIHYSYGVGNTEKGDIPDGYEASRPSHENFSLILVSATPSQSSVYFCASGVGGTLYFGAGTRLSVL

SEQ ID NO: 26 - protein, 110 aa, Mus musculus
EAVVTQSPRNKVAVTGEKVTLSCNQTNNHNNMYWYRQDTGHGLRLIHYSLGIHSTEKGDIPDGYKASRPSQENFSLILELATPSQTSVYFCASGGGGTLYFGAGTRLSVL

SEQ ID NO: 27 - protein, 110 aa, Mus musculus
EAVVTQSPRNKVAVTGEKVTLSCNQTNNHNNMYWYRQDTGHELRLIHYSLGIHNVRRGDIPDGYKASRPSQENFSLILELATPSQTSVYFCASGGGGTLYFGAGTRLSVL

SEQ ID NO: 28 - protein, 110 aa, Mus musculus
EAVVTQSPRNKVAVTGEKVTLSCNQTNNHNNMYWYRQDTGHELRLIFMSHGIHNVEKGDIPDGYKASRPSQENFSLILELATPSQTSVYFCASGGGGTLYFGAGTRLSVL

SEQ ID NO: 29 - protein, 110 aa, Mus musculus
EAVVTQSPRNKVAVTGEKVTLSCKQTNNHNNMYWYRQDTGHELRLIFMSHGIRNVEKGDIPDGYKASRPSQENFSLILELATPSQTSVYFCASGGGGTLYFGAGTRLSVL

SEQ ID NO: 30 - protein, 111 aa, Mus musculus
EAVVTQSPRNKVAVTGEKVTLSCKQTNTYLNNMYWYRQDTGHELRLIFMSHGIRNVEKGDIPDGYKASRPSQENFSLILELATPSQTSVYFCASGGGGTLYFGAGTRLSVL

SEQ ID NO: 31 - protein, 111 aa, Mus musculus
EAVVTQSPRNKVAVTGEKVTLSCKQTCSYLDNMYWYRQDTGHELRLIFMSHGIRNVEKGDIPDGYKASRPSQENFSLILELATPSQTSVYFCASGGGGTLYLGAGTRLSVL

SEQ ID NO: 32 - protein, 111 aa, Mus musculus
EAVVTQSPRNKVAVTGEKVTLSCKQTGSYFDNMYWYRQDTGHELRLIFMSHGIRNVEKGDIPDGYKASRPSQENFSLILELATPSQTSVYFCASGGGGTLYFGAGTRLSVL

SEQ ID NO: 33 - protein, 111 aa, Mus musculus
EAVVTQSPRNKVAVTGEKVTLSCKQTKGYNDNMYWYRQDTGHELRLIFMSHGIRNVEKGDIPDGYKASRPSQENFSLILELATPSQTSVYFCASGGGGTLYFGAGTRLSVL

SEQ ID NO: 34 - protein, 111 aa, Mus musculus
EAVVTQSPRNKVAVTGEKVTLSCKQTNTYWNNMYWYRQDTGHELRLIFMSHGIRNVEKGDIPDGYKASRPSQENFSLILELATPSQTSVYFCASGGGGTLYFGAGTRLSVL

SEQ ID NO: 35 - protein, 111 aa, Mus musculus
EAVVTQSPRNKVAVTGEKVTLSCKQTNSYFDNMYWYRQDTGHELRLIFMSHGIRNVEKGDIPDGYKASRPSQENFSLILELATPSQTSVYFCASGGGGTLYFGAGTRLSVL

SEQ ID NO: 36 - protein, 111 aa, Mus musculus
EAVVTQSPRNKVAVTGEKVTLSCKQTNSYFNNMYWYRQDTGHELRLIFMSHGIRNVEKGDIPDGYKASRPSQENFSLILELATPSQTSVYFCASGGGGTLYFGAGTRLSVL

SEQ ID NO: 37 - protein, 111 aa, Mus musculus; identical to SEQ ID NO: 36

SEQ ID NO: 38 - protein, 110 aa, Mus musculus
EAVVTQSPRNKVAVTGEKVTLSCKQRDRWNNMYWYRQDTGHELRLIFMSHGIRNVEKGDIPDGYKASRPSQENFSLILELATPSQTSVYFCASGGGGTLYFGAGTRLSVL

SEQ ID NO: 39 - protein, 111 aa, Mus musculus
EAVVTQSPRNKVAVTGEKVTLSCKQTNSYLDNMYWYRQDTGHELRLIFMSHGIRNVEKGDIPDGYKASRPSQENFSLILELATPSQTSVYFCASGGGGTLYFGAGTRLSVL

SEQ ID NO: 40 - protein, 110 aa, Mus musculus; identical to SEQ ID NO: 38

SEQ ID NO: 41 - protein, 110 aa, Mus musculus
EAVVTQSPRNKVAVTGEKVTLSCKQTNNHNNMYWYRQDPGHELRLIFMSHGIRNVEKGDIPDGYKASRPSQENFPLILELATPSQTSVYFCASGGGGTLYFGAGTRLSVL

SEQ ID NO: 42 - protein, 111 aa, Mus musculus
EAVVTQSPRNKVAVTGEKVTLSCKQTNSYFPNMYWYRQDTGHELRLIFMSHGIRNVEKGDIPDGYKASRPSQENFSLILELATPSQTSVYFCASGGGGTLYFGAGTRLSVL

SEQ ID NO: 43 - protein, 111 aa, Mus musculus
EAVVTQSPRNKVAVTGEKVTLSCKQTIGYLNNMYWYRQDTGHELRLIFMSHGIRNVEKGDIPDGYKASRPSQENFSLILELATPSQTSVYFCASGGGGTLYFGAGTRLSVL

SEQ ID NO: 44 - protein, 110 aa, Mus musculus; identical to SEQ ID NO: 38

SEQ ID NO: 45 - protein, 110 aa, Mus musculus; identical to SEQ ID NO: 23

SEQ ID NO: 46 - protein, 110 aa, Mus musculus; identical to SEQ ID NO: 24

SEQ ID NO: 47 - protein, 110 aa, Mus musculus
EAVVTQSPRNKVAVTGEKVTLSCNQTNNHNNMYWYRQDTGHELRLIHYSLGIHSTEKGDIPDGYKASRPSQENFSLILELATPSQTSVYFCASGGGGTLYFGAGTRLSVL

SEQ ID NO: 48 - protein, 110 aa, Mus musculus
EAVVTQSPRNKVAVTGEKVTLSCNQTNNHNNMYWYRQDTGHELRLIHYSLGIRSTEKGDIPDGYKASRPSQENFSLILELATPSQTSVYFCASGGGGTLYFGAGTRLSVL

SEQ ID NO: 49 - protein, 110 aa, Mus musculus
EAVVTQSPRNKVAVTGEKVTLSCNQTNNHNNMYWYRQDTGHGLRLIHYSNGVRSTEKGDIPDGYKASRPSQENFSLILELATPSQTSVYFCASGGGGTLYFGAGTRLSVL

SEQ ID NO: 50 - protein, 110 aa, Mus musculus
EAVVTQSPRNKVAVTGEKVTLSCNQTNNHNNMYWYRQDTGHGLRLIHYSYGAGHIGKGDIPDGYKASRPSQENFSLILELATPSQTSVYFCASGGGGTLYFGAGTRLSVL

SEQ ID NO: 51 - protein, 110 aa, Mus musculus
EAVVTQSPRNKVAVTGEKVTLSCNQTNNHNNMYWYRQDTGHGLRLIYYSYGAGHLLSGDIPDGYKASRPSQENFSLILELATPSQTSVYFCASGGGGTLYFGAGTRLSVL

SEQ ID NO: 52 - protein, 110 aa, Mus musculus
EAVVTQSPRNKVAVTGEKVTLSCNQTNNHNNMYWYRQDTGHGLRLIYYSYGAGHVGKGDIPDGYKASRPSQENFSLILELATPSQTSVYFCASGGGGTLYFGAGTRLSVL

SEQ ID NO: 53 - protein, 110 aa, Mus musculus
EAVVTQSPRNKVAVTGEKVTLSCNQTNNHNNMYWYRQDTGHELRLIHYSLGIRNVISGDIPDGYKASRPSQENFSLILELATPSQTSVYFCASGGGGTLYFGAGTRLSVL

SEQ ID NO: 54 - protein, 110 aa, Mus musculus; identical to SEQ ID NO: 27

SEQ ID NO: 55 - protein, 110 aa, Mus musculus
EAVVTQSPRNKVAVTGEKVTLSCNQTNNHNNMYWYRQDTGHELRLIHYSLGIRNVRPGDIPDGYKASRPSQENFSLILELATPSQTSVYFCASGGGGTLYFGAGTRLSVL

SEQ ID NO: 56 - protein, 110 aa, Mus musculus
EAVVTQSPRNKVAVTGEKVTLSCNQTNNHNNMYWYRQDTGHELRLIHYSLGIHNVEKGDIPDGYKASRPSQENFSLILELATPSQTSVYFCASGGGGTLYFGAGTRLSVL

SEQ ID NO: 57 - protein, 110 aa, Mus musculus; identical to SEQ ID NO: 28

SEQ ID NO: 58 - protein, 110 aa, Mus musculus
EAVVTQSPRNKVAVTGEKVTLSCNQTNNHNNMYWYRQDTGHELRLIFGSYGIHNVEKGDIPDGYKASRPSQENFSLILELATPSQTSVYFCASGGGGTLYFGAGTRLSVL

SEQ ID NO: 59 - protein, 110 aa, Mus musculus
EAVVTQSPRNKVAVTGEKVTLSCNQTNNHNNMYWYRQDTGHELRLIFRSLGIRNVEKGDIPDGYKASRPSQENFSLILELATPSQTSVYFCASGGGGTLYFGAGTRLSVL

SEQ ID NO: 60 - protein, 110 aa, Mus musculus
EAVVTQSPRNKVAVTGEKVTLSCNQTNNHNNMYWYRQDTGHELRLIFASLGIHNVEKGDIPDGYKASRPSQENFSLILELATPSQTSVYFCASGGGGTLYFGAGTRLSVL

SEQ ID NO: 61 - protein, 110 aa, Mus musculus
EAVVTQSPRNKVAVTGEKVTLSCNQTNNHNNMYWYRQDTGHELRLIFRSLGIHNVEKGDIPDGYKASRPSQENFSLILELATPSQTSVYFCASGGGGTLYFGAGTRLSVL

SEQ ID NO: 62 - protein, 110 aa, Mus musculus; identical to SEQ ID NO: 29

SEQ ID NO: 63 - protein, 110 aa, Mus musculus
EAVVTQSPRNKVAVTGEKVTLSCSQTNNHNNMYWYRQDTGHELRLIFMSHGIRNVEKGDIPDGYKASRPSQENFSLILELATPSQTSVYFCASGGGGTLYFGAGTRLSVL

SEQ ID NO: 64 - protein, 110 aa, Mus musculus
EAVVTQSPRNKVAVTGEKVTLSCSQTNNHNNMYWYRQDTGHELRLIFASLGIHNVEKGDIPDGYKASRPSQENFSLILELATPSQTSVYFCASGGGGTLYFGAGTRLSVL

SEQ ID NO: 65 - protein, 110 aa, Mus musculus
EAVVTQSPRNKVAVTGEKVTLSCSQTNNHNNMYWYRQDTGHELRLIFRSLGIRNVEKGDIPDGYKASRPSQENFSLILELATPSQTSVYFCASGGGGTLYFGAGTRLSVL

SEQ ID NO: 66 - protein, 111 aa, Mus musculus; identical to SEQ ID NO: 32

SEQ ID NO: 67 - protein, 111 aa, Mus musculus; identical to SEQ ID NO: 33

SEQ ID NO: 68 - protein, 111 aa, Mus musculus; identical to SEQ ID NO: 34

SEQ ID NO: 69 - protein, 111 aa, Mus musculus; identical to SEQ ID NO: 36

SEQ ID NO: 70 - protein, 111 aa, Mus musculus; identical to SEQ ID NO: 35

SEQ ID NO: 71 - protein, 110 aa, Mus musculus; identical to SEQ ID NO: 38

SEQ ID NO: 72 - protein, 111 aa, Mus musculus; identical to SEQ ID NO: 30

SEQ ID NO: 73 - protein, 111 aa, Mus musculus
EAVVTQSPRNKVAVTGEKVTLSCKQTCSYLDNMYWYRQDTGHELRLIFMSHGIRNVEKGDIPDGYKASRPSQENFSLILELATPSQTSVYLCASGGGGTLYFGAGTRLSVL